(19) United States
(12) Patent Application Publication
(10) Pub. No.: US 2025/0027901 A1
(43) Pub. Date: Jan. 23, 2025

(54) DIRECTLY FUNCTIONALIZED ELECTROCHMICAL TRANSISTEORS, AND CONVECTION DRIVEN ULTRA-RAPID DETECTION OF BIOMARKERS USING TRANSISTORS

(71) Applicant: King Abdullah University of Science and Technology, Thuwal (SA)

(72) Inventors: Sahika Inal, Thuwal (SA); Stefan T. Arold, Thuwal (SA); Raik Grunberg, Thuwal (SA); Shofarul Wustoni, Thuwal (SA); Keying Guo, Thuwal (SA); Anil Koklu, Thuwal (SA); Miriam Escarlet Diaz Galicia, Thuwal (SA)

(21) Appl. No.: 18/711,844

(22) PCT Filed: Nov. 18, 2022
(86) PCT No.: PCT/IB2022/061164

§ 371 Date: May 20, 2024

Related U.S. Application Data
(60) Provisional application No. 63/280,887, filed on Nov. 18, 2021.
(60) Provisional application No. 63/283,447, filed on Nov. 27, 2021.
(60) Provisional application No. 63/318,188, filed on Mar. 9, 2022.

(51) Int. Cl. G01N 27/414; G01N 27/327; G01N 33/543; G01N 33/569

(52) U.S. Cl. CPC G01N 27/4145; G01N 27/3276; G01N 27/3278; G01N 33/5438; G01N 33/56983; G01N 2333/165; G01N 2469/10

(57) ABSTRACT

Devices and methods of analyte detection using AC electrokinetic/electrohydrodynamic forces combined with an OECT-based immunosensor are disclosed. An analyte binding agent, for example, a nanobody-functionalized organic electrochemical transistor (OECT) is incorporated with the micro-stirring effect of alternating current electrothermal flow (ACET) for the ultrarapid detection of single-molecule-to-nanomolar levels of the analyte. The ACET flow is induced by a biased AC electrical field can rapidly convect the analyte onto concentric gate electrodes within a minute, and the analyte is captured via recognition units that bind the analyte binding agent while sweeping nonspecific ally bound analyte away from the surface.

### CROSS-REFERENCE TO RELATED APPLICATIONS

This application claims the benefit of and priority to U.S. Provisional Application No. 63/280,887 filed Nov. 18, 2021, U.S. Provisional Application No. 63/283,447 filed Nov. 27, 2021, and U.S. Provisional Application No. 63/318,188 filed Mar. 9, 2022, which are hereby incorporated by reference in their entirety.

### FIELD OF THE INVENTION

This invention is generally in the field of organic electrochemical transistors (OECT)-based immunosensor devices and methods useful in detecting an analyte of interest in a sample.

### BACKGROUND OF THE INVENTION

Reliable biomolecular diagnostics are an important tool for early detection of diseases, particularly for preventing the outbreak of infectious diseases such as HIV, Ebola and recently widespread coronavirus (SARS-CoV-2) which has caused one million fatalities worldwide, at the time of this writing. Early-stage detection is particularly useful to identify and isolate infected patients without symptoms before spreading the disease. Thus, it is crucial to develop a detection method that can offer rapid, easy-use and accurate results.

Detection methods such as RRT-PCT have inherent limitations, such as the labor-intensive sample preparation in a laboratory setting, which increases the test turn-around time as well as inapplicability to minute sample volumes. Moreover, sample transportation and complex sample preparation steps prior to testing might also reduce clinical sensitivity, resulting in false-negative results.

Organic electrochemical transistors (OECTs) have attracted attention as a promising alternative biosensing technology that can surpass other state-of-the-art electrical biosensors approaches such as Field Effect Transistors (FETs). OECTs allow biosensing applications in an aqueous environment with low voltage operation (<1V) and high amplification (Lin and Yan, 2012). OECTs detect target analytes by monitoring the capacitive or faradaic changes occurring in the device interfaces. To date, the actual biosensing signal is most commonly provided from enzymatic redox reactions that are fueled by a specific analyte. Analyte binding to an enzyme induces a redox reaction which then causes a potential drop across the device interface which is then amplified into a change of electrical current flowing from transistor source to drain terminals (Bernard et al., 2008; Ohayon et al., 2020). An important advantage of OECT biosensors is that their selectivity can be tuned by incorporating suitable bio-recognition elements without the need for fluorescent-, radio- or other labels (Wustoni et al., 2019; Wustoni et al., 2020).

Diagnostic tools that use the enzymatic (mostly collateral cleavage) activity of CRISPR-Cas proteins are programmable to different targets, and some have been integrated with portable detection methods such as lateral-flow strips. However, they still require time-consuming sample pre-processing steps including target amplification and/or RNA extraction to reach the required attomolar sensitivity. The time from taking the sample to having the final result is therefore much longer than 15 min. While these tests can lower the cost and complexity of molecular testing, they seem to have very limited advantages over, for example, established RT-LAMP or more streamlined RT-PCR solutions. There is a need for RNA-diagnostic tool that achieves fast response, and high sensitivity, in raw unprocessed samples.

OECT-based immunosensors have been developed, in which p-type accumulation mode material (p(g0T2-g6T2)) was used as a channel material, demonstrating single-protein molecule sensitivity and an extended dynamic range. The VHH-72 nanobody-OECT biosensor detected specific proteins from unprocessed human samples in ambient conditions after 10 min of incubation by manually pipetting for 30 s every 3 min. This biosensor involves a tedious pipetting step for accelerating the protein transport to the sensor, resulting in a relatively short incubation time, which is not possible with diffusion-based transport. Thus, a highly automated particle transport approach for rapid screening and field-use applications is essential.

It is an object of the present invention to provide OECT-based biosensors which allow for decreased incubation time and/or improved sensitivity.

It is also an object of the present invention to provide methods for making OECT-based immunosensor for use in diagnostic assays.

It is also an object of the present invention to provided improved methods for detecting analyte in a sample using OECT-based biosensors.

### SUMMARY OF THE INVENTION

OECT-based biosensors are provided herein, as well as methods of making and methods of use thereof. The biosensor includes an OECT and a biorecognition layer. The OECT is preferably an electrolyte gated transistor, that is, a three-terminal electronic device which includes a source electrode, a drain electrode, a channel, and a gate electrode. In some preferred embodiments, the biorecognition layer is integrated on the gate electrode of the OECT.

In some forms, the biorecognition layer includes one biological self-assembled monolayer (SAM) formed through a specific biological autocatalytic coupling strategy (herein, Bio-SAM), of biological molecules, as disclosed herein, and preferably excludes the use of a layer of SAM formed using organic molecules i.e., Chem-SAM, which can be formed by self-assembly of the organic molecules such as thiol containing organic molecules as disclosed in PCT/IB2021/055981. The Bio-SAM includes a biorecognition element, a protein which is not a whole antibody, a fusion protein which does not include a while antibody, or a CRISPR protein (e.g., a ribonucleoprotein complex Cas:gRNA). The biorecognition element includes a binding partner for an analyte of interest, preferably, a pathogen or component thereof. In one preferred embodiment, the biorecognition element can bind to the SARS-CoV-2 receptor binding domain (RBD) or Spike protein (S1), which is preferably, a nanobody (for example, VHH72) or related antibody fragment, or a CRISPR protein (such as a ribonucleoprotein complex Cas:gRNA).

The organization of the biorecognition element on the OECT surface can be represented by the general formula:

L1-AP1:AP2-L2-B Formula I

Where L1 is a first linker, AP1 is the first peptide binding partner harboring a specific biological recognition sequence; AP2 is the second peptide binding partner recognizing this sequence on AP1; AP1 and AP2 are binding partners, preferably, covalent binding partners; however, they can be members of an affinity pair; L2 is a second linker and B is the biorecognition element of the biosensor. AP2-L2-B can be produced as a single fusion protein without the need for chemical modification. The OECT surface is modified with L1-AP1 via L1. L1 is preferably a peptide sequence including a cysteine residue at its N- or C-terminus which provides a free SH group for direct coupling of L1 to the surface of the gate electrode. The OECT surface is preferably, not modified with a simple organic alkane thiol or derivative thereof, such as 1,6-hexanedithiol (HDT), between the OECT and L1, i.e. L1 is directly coupled to the OECT (referred to herein as directly functionalized OECT) and provides a specific (biologically derived) recognition sequence AP1 for AP2 rather than an unspecific reactive group. In other words, the electrode that contains the biorecognition layer formed thereon does not contain free organic molecules, i.e., organic molecules self-assembled on the electrode surface but are not attached to L1-AP1 and thus not attached to a biorecognition element. For example, the gate electrode modified with the biorecognition layer does not contain free cysteines that are not linked to the first linker L1, such as a peptide sequence. Binding or coupling of AP1 and AP2 results in a biologically self-assembled monolayer, herein, Bio-SAM. Thus, the biorecognition layer includes Linker L1 and a Bio-SAM. In one particular embodiment, AP1 is spyTag and AP2 is spyCatcher.

The biorecognition layer is preferably integrated on the gate electrode of the OECT.

The channel of the OECT may be formed directly from a conducting polymer or by incorporating a conducting polymer on the surface of a conductive substrate (such as by spin-coating the conducting polymer on the surface of a metal electrode). Any suitable conducting polymers can be used for the channel of the OECT. In some embodiments, the conducting polymer for the channel of the OECT is a p-type polymer. In some embodiments, the conducting polymer for channel of the OECT is an n-type polymer. In some embodiments, the conducting polymer for the channel of the OECT is poly(3,4-ethylenedioxythiophene) doped with poly(styrene sulfonate) (PEDOT:PSS) or any other mixed (ionic and electronic) semiconductor. In some embodiments, the conducing polymer for the channel of the OECT is p(g0T2-g6T2). In some embodiments, the conducting polymer for the channel of the OECT is p(g3C2T2-T). In some embodiments, the conducting polymer for the channel of the OECT is p(C6NDI-T). Depending on channel material, the OECT can operate in depletion or in accumulation mode.

Also disclosed are methods for making OECT devices containing a binding partner for any analyte of interest, for example an antigen from any pathogen. The method includes functionalizing the gate electrode of the OECT with a biorecognition layer which includes a binding partner for the analyte of interest, as follows: (i) contacting at least a portion of the surface of the gate electrode with a first solution containing a peptide sequence which preferably includes a cysteine residue at its N- or C-terminus, and a first peptide binding partner and (ii) contacting the first binding partner-modified surface with one or more solutions containing a linker or linker elements and the binding partner for in the analyte. The method for functionalizing the gate electrode of the OECT excludes a step of contacting at least a portion of the surface of the gate electrode with a solution containing a thiol containing organic molecules, such as cysteines, prior to step (i).

Referring to Formula I (L1-AP1:AP2-L2-B), at least a portion of the surface of the gate electrode is contacted with a solution containing a peptide sequence (L1) which preferably includes a free SH group that is most conveniently provided by a cysteine residue at its N- or C-terminus, and a first peptide binding partner (AP1) to form a first binding partner layer on the surface of electrode, and contacting this first binding partner layer with a composition containing AP2-L2-B under conditions resulting in conjugation of AP1 and AP2 to form a biologically self-assembled layer, and referred to herein as Bio-SAM. In one preferred embodiment, the AP1 and AP2 are the spyTag and spyCatcher, respectively. Autocatalytic covalent binding between the spyTag/spyCatcher pair orients the analyte binding partner on the surface of the OECT device. In a particularly preferred embodiment, the method includes contacting the OECT surface with a first solution containing spy Tag (one partner of spyTag/spyCatcher pair) followed by contacting with a second solution containing spyCatcher, the second partner of the spyTag/spyCatcher pair which is in turn linked to the binding partner for the analyte of interest. In one preferred embodiment, the analyte of interest is the SARS-CoV-2 RBD and its binding partner is a specific nanobody.

Also disclosed are methods for identifying an analyte of interest using directly functionalized-OECT. One embodiment includes contacting the sample with the directly functionalized OECT-based biosensor disclosed herein, which includes a binding partner for the analyte of interest. Another embodiment includes using AC electrokinetic/electrohydrodynamic forces, as described herein.

Devices and methods of analyte detection using AC electrokinetic/electrohydrodynamic forces combined with an OECT-based immunosensor are disclosed. Typically, the gate electrode of the OECT-based immunosensor is surrounded by a conductive element for applying an AC electrokinetic/electrohydrodynamic force in the device.

One embodiment provides a directly functionalized OECT as described above and another embodiment provides an indirectly nanobody-functionalized OECT (such as those described in PCT/IB2021/055981), either embodiment incorporated with the conductive element capable of providing a micro-stirring effect of alternating current electrothermal flow (ACET) for the ultra-rapid detection of single-molecule-to-nanomolar levels of a pathogen such as the severe acute respiratory syndrome coronavirus 2 (SARS-CoV-2) in complex bodily fluids (such as saliva, plasma, serum and blood). The ACET flow induced by a biased AC electrical field applied to the conductive element can rapidly convect the analyte onto concentric gate electrodes within a minute, and antigens from the pathogen, such as SARS-CoV-2 spike proteins (S1) or receptor-binding domains (RBDs) are captured via recognition units (nanobody) while sweeping nonspecific ones away from the surface.

### BRIEF DESCRIPTION OF THE DRAWINGS

FIG. 1A shows a schematic illustration of the SpyDirect nanobody-functionalized OECT sensor. The gate electrode is directly biofunctionalized with a spy Tag-linked cysteine peptide and a spyCatcher-linked nanobody. BSA is used as a blocking agent. The sensor is incubated with saliva. FIG. 1B shows a schematic illustrating operation of the OECT sensor. The bare sensor gate or the incubated sensor gate is mounted on the top of the channel for signal acquisition. FIG. 1C shows the output signal of the OECT sensor before and after exposure to COVID-19 positive saliva. NR=|(g_{mQ}−g_{mQ})|g_{mQ}

FIGS. 2A and 2B show QCMD (FIG. 2A) and AFM (FIG. 2B) characterization of the SpyCatcher-nanobody biofunctionalization of the gate electrode.

FIGS. 3A-3G show electrochemical characterization and XPS spectra of the biofunctionalized Au gate electrode: cyclic voltammogram (FIG. 3A), Bode plot (solid lines and dotted lines are corresponding to the magnitude and the phase of the impedance, respectively) (FIG. 3B), Nyquist plot of the gold electrode before and after the subsequent functionalization with SpyTag-cysteine peptide, and the nanobody-SpyCatcher (FIG. 3C; inset is the equivalent circuit model used to fit the impedance spectra), calculated charge transfer resistance (Rct) and electric double layer capacitance (Cdl) change of the electrode (FIG. 3D; measurements were operated in 10 mM [Fe(CN)6]3−/4− in 10 mM PBS, pH 7.4), and high-resolution XPS spectra for Au 4f, C1s and N1s during each modification step (FIGS. 3E-3G). The nanobody-SpyCatcher buffer contained BSA.

FIGS. 4A-4E show detection of SARS-CoV-2 using SpyDirect SARS-CoV-1 nanobody sensor. FIG. 4A shows the transfer characteristics of the accumulation mode OECT after incubation of SARS-CoV-2 spike with different concentrations in sequence. FIG. 4B shows the transconductance as function of gate voltage for the OECT sensors with nanobody-functionalized gate electrode after incaution. FIG. 4C shows the normalized response (NR) to SARS-CoV-2 spike and non-target (lysozyme) in saliva. Error bars represent the standard deviation calculated from at least 3 gate electrodes. FIG. 4D shows the schematic for OECT interfaces. Ionic circuit used to model OECT consisting of capacitors corresponding to the gate, C_{G}, and channel, C_{CH}, respectively, and a resistor corresponding to the electrolyte, RE. FIG. 4E shows the densing mechanism. Schematic for gate capacitance decreases upon protein binding (C_{G}→C′_{G,eff}).

FIG. 5A-5E show random detection of SARS-CoV-2 spike protein in saliva and untreated wastewater. FIG. 5A shows the random detection of SARS-CoV-2 spike in saliva with SpyDirect SARS-CoV-1 nanobody gates while using GFP nanobody gates as control. FIG. 5B shows the detection of SARS-CoV-2 spike in untreated wastewater. FIGS. 5C and 5D show the long-term stability test of HDT SAM-based gates (FIG. 5C) and SpyDirect gates (FIG. 5D) to SARS-CoV-2 S1 (1.2 pM) in saliva. The gates were stored in 10 mM PBS, pH 7.4 at 4° C. for 3, 7 and 14 days before use. FIG. 5E shows the sensor response of SpyDirect gates prepared from dried cysteine peptide. These cysteine peptide-modified gates were stored for 7 days in ambient condition before the nanobody immobilization.

FIGS. 6A-6F show the detection of SARS-CoV-2 pseudotyped lentivirus in human saliva, universal transport medium (UTM) and untreated wastewater. FIG. 6A shows the background response of SpyDirect- and HDT SAM-sensor to human saliva, UTM and untreated wastewater. FIG. 6B shows the SpyDirect sensor response to SARS-CoV-2 pseudotyped lentivirus and non-target in human saliva. FIG. 6C-6F show the sensor response to SARS-CoV-2 pseudotyped lentivirus in UTM (FIG. 6C) or wastewater (FIG. 6E) using SpyDirect- and sensor response to SARS-CoV-2 pseudotyped lentivirus in UTM (FIG. 6D) or wastewater (FIG. 6F) using HDT SAM-based nanobody gates. GFP nanobody gates were used as controls. Error bars represent the standard deviation calculated from at least 3 gate electrodes.

FIGS. 7A-7H show the electrochemical and chemical stability test of SARS-CoV-1 nanobody gates. FIGS. 7A and 7B show the charge transfer resistance (Rct) of SpyDirect-based (FIG. 7A) and HDT SAM-based (FIG. 7B) gates. FIGS. 7C-7H show XPS spectra of C1s, O1s and N1s of SpyDirect-based (FIGS. 7C-7E) and HDT SAM-based (FIG. 7F-7H) gates before and after 7 days' storage. The SARS-CoV-1 nanobody functionalized gates were kept in 10 mM PBS, pH 7.4 at 4° C., and the data were acquired on day 0, 3 and 7, respectively (n=3). The OECT planar gates with well-defined area (500 μm×500 μm) were functionalized with SARS-CoV-1 nanobody using either SpyDirect method or HDT SAM as linker, then those SARS-CoV-1 nanobody gates were used to monitor their long-term electrochemical stability. The impedance measurements were operated in 10 mM [Fe(CN)₆]^{3-/4-} in 10 mM PBS, pH 7.4. The Randles circuit model was used to fit the impedance spectra.

FIGS. 8A-8B show stability of gate functionalization. Charge-transfer resistance of nanobody-coated gates was measured after storage in PBS for 0, 3 and 7 days. (FIG. 8A) Gates prepared with the spyDirect (Cys-peptide) SAM show a stable low charge transfer resistance over time. (FIG. 8B) Gates prepared using the HDT-SAM show a strong increase of charge-transfer resistance over time indicating deterioration of the surface by, e.g., oxidation processes.

FIG. 8A shows the circular gate electrode is surrounded by a gold layer to apply the alternating current electrothermal flow (ACET). Scale bar: 100 μm. FIG. 8B is a schematic illustrating the architecture of the biorecognition unit on the gate electrode. The SpyTag peptide is chemically coupled to the HDT monolayer assembled on the gold electrode. The nanobody-SpyCatcher fusion protein is attached to this chemical layer through the autocatalytic formation of a covalent SpyCatcher-SpyTag bond. Three Ty1 nanobody structures are shown in complex with the trimeric SARS-CoV-2 spike protein (shown partially in grey, based on PDB entry 6ZXN). FIG. 8C is a schematic illustrating the operation steps of the ACET enhanced nanobody-OECT biosensor. 10 μL of the sample solution is introduced on top of the concentric gate electrode. A function generator supplies an AC potential and induces ACET to concentrate the target molecules (e.g., spike protein) on the surface with immobilized nanobodies. This process takes 2 min. The electrode is then rinsed with phosphate-buffered saline (PBS), flipped, and mounted on top of the channel for signal acquisition.

FIGS. 9A-9F show characterization of the Ty1-nanobody-functionalized concentric gate electrode. FIG. 9A shows the Nyquist plot of the Au electrode before and after the subsequent functionalization with HDT, Spy Tag peptide, and SpyCatcher/nanobody. FIG. 9B shows the Randles equivalent circuit model used to extract the solution resistance (Rsol), electric-double-layer capacitance (CEDL), charge-transfer resistance (Rct), and Warburg resistance (W). FIG. 9C shows the calculated values of the electrical components after each functionalization step. FIG. 9D shows the real-time monitoring of the Ty1-nanobody immobilization on the Au surface using QCM-D (top: frequency changes, bottom: mass of the layers on the crystal). The gold QCM-D sensor comprising an HDT layer was subjected to the two-step functionalization protocol consisting of the SpyTag peptide coupling followed by the SpyCatcher/nanobody immobilization. PBS was used to remove unbound species from the surface. FIGS. 9E and 9F show the high-resolution C1s (FIG. 9E) and N1s (FIG. 9F) XPS spectra of the gold surface after the immobilization of different functional layers.

FIGS. 10A-10I show the biosensor performance of the p-type and the n-type OECT. FIG. 10A-10D show the transfer curves of p(g3C₂T2-T) (FIGS. 10A and 10B) and p(C₆NDI-T) (FIGS. 10C and 10D) channels recorded with the Ty1-nanobody electrodes (FIGS. 10A and 10C) and GFP-nanobody electrodes (FIGS. 10B and 10D). The gate electrodes were incubated with solutions containing various concentrations of SARS-CoV-2 S1 under ACET. The arrows in (FIG. 10A) and (FIG. 10C) indicate the increase in the protein concentration of the solution from 1×10⁻¹⁸ m to 1×10⁻⁹ m. FIG. 10E-10G show the normalized response (NR) of p(g₃C₂T2-T) OECT determined at VG=−0.1 V and VD=−0.1 V (FIG. 10E) and p(C₆NDI-T) OECT determined at VG=0.5 V and VG=0.9 V, respectively (VD=0.1 V) (FIGS. 10F and 10G). The error bars represent the standard deviation calculated from at least three gate electrodes. FIGS. 10H and 10I show the NR of p(C6NDI-T) OECTs operated with the same gate electrodes but without ACET, reported at VG=0.5 V and VG=0.9 V, respectively (VD=0.1 V). FIG. 10J shows a schematic of the equivalent circuit model used to extract the voltage drop at the gate/electrolyte interface (V_{G}^{drop}). FIG. 10K shows a representative Bode plot of the Au gate electrode and the channel materials biased at V=|0.5|V vs Ag/AgCl to their doped states. FIG. 10L shows the V_{G}^{drop} vs gate capacitance (Cg) that decreases as the target molecule concentration binding on its surface increases. The analytical model is considered for four types of devices based on different gate-to-channel capacitance ratios: 1) Cg=0.1 Cch, 2) Cg=Cch, 3) Cg=10 Cch, and 4) Cg=100 Cch.

FIGS. 11A-11G show the performance of the ACET-assisted n-type OECT sensor in saliva and benchmarking its performance. FIGS. 11A and 11B show the response of p(C6NDI-T) channel gated with the Ty1- and GFP-nanobody-functionalized electrodes to various concentrations of SARS-CoV-2 S1 in saliva, recorded at VG=0.5 V (FIG. 11A) and VG=0.9 V (FIG. 11B), respectively. The saliva was diluted with lysis buffer at a 1:4 ratio. FIGS. 11C and 11D show the response of the n-type OECTs to randomly selected saliva samples containing various amounts of SARS-CoV-2 S1. Each data point represents the response of a single gate electrode. The data in these two plots are acquired using two sets of electrodes prepared at the same time. The small circles represent the negative control measurements conducted with GFP-nanobody-functionalized gate electrodes while the large circles represent the recordings from the Ty1-nanobody-functionalized gate electrodes. The data recorded at VG=0.9 V and VD=0.1 V are presented. FIG. 11E shows the NR difference between the Ty1- and GFP-nanobody gated OECTs. FIGS. 11F and 11G show the power consumption vs operating voltage plot (FIG. 11F) and the limit of detection (LOD), dynamic range, and incubation time (FIG. 11G) of the OECT-based immunosensor (represented with stars) compared with other transistor-based immunosensors (see Table 3 for the list of references).

FIGS. 12A and 12B show schematics of an exemplary reusable OECT (FIG. 12A) and an exemplary reader device (FIG. 12B) for measuring signals.

FIGS. 13A and 13B show schematics of an exemplary point-of-care (“POC”) OECT (FIG. 13A) and exemplary operation steps for using the POC OECT (FIG. 13B).

FIG. 14 shows an exemplary high-throughput device, in which 96 gate electrodes are used for simultaneous measurements. The gate holder is transferred between different plates containing e.g. patient sample, wash buffer, electrolyte shaking speeds up target capture by nanobody surface (otherwise severe diffusion limitation) for measurement. The holder can be placed on top of a custom measurement plate containing the OECT channels where electrical contact can be established with all the gates from the top.

### DETAILED DESCRIPTION OF THE INVENTION

Devices for sensitive and rapid analyte detection using an OECT sensor are provided. The devices can be used for rapid detection of an infection by a pathogen such as a SARS-CoV-2 by detecting the presence of an antigen (analyte) specific for the pathogen, using a directly biofunctionalized OECT sensor, where the OECT sensor is functionalized to include a binding partner for the antigen of interest.

The disclosed directly functionalized OECT sensors are based on an unexpected discovery that a modified and simplified immobilization strategy using an all protein-based Bio-SAM which eliminates the underlying Chem-SAM (which is made of organic molecules, such as alkane thiols and derivatives thereof), improves analyte detection using the OECT sensor. Exemplified herein using SpyTag and SpyCatcher, a cysteine-terminated peptide with SpyTag (and without any non-biological chemical modification) is directly coupled to the OECT sensor surface (such as the gate electrode surface of the OECT) and then used to immobilize anti-SARS-CoV nanobody on the OECT sensor without involving any organic chemicals such as HDT, which self-assemble into a monolayer at the OECT surface (Chem-SAM) and then attach to a peptide with SpyTag. Using the direct biofunctionalization method, the electrode surface that contains the binding partner for the analyte of interest formed thereon does not contain free organic molecules, i.e., organic molecules self-assembled on the electrode surface but are not linked to a peptide with SpyTag.

Compared to the Chem-SAM based nanobody OECT sensor, the cysteine peptide linked nanobody-OECT sensors achieve a higher packing density of coupled nanobody and thus a higher binding capacity for the analyte of interest (such as at least 1.5 times higher or at least 2 times higher). For example, the packing density of peptide is up to 82×10¹² per cm² SpyDirect-peptide (compared to 32×10¹² maleimide-peptide per cm² using Chem SAM) and packing density of nanobody is up to 7.8×10¹² per cm² (compared to 6.5×10¹² per cm² using Chem SAM). They show a reduction in background noise level in universal transport medium (UTM) as well as in raw saliva, a lower limit of detection (LOD) with broad detection window (such as a LOD as low as 6×10⁻²² M with 10 orders of magnitude of detection window) and are insensitive to contaminants such as dithiothreitol (DTT). They also show an improved long-term stability. For example, the Rct average value of the SpyDirect gates shows negligible change after 3-day storage in PBS, while a decrease of 12.5% after 7 days' storage. In contrast, the HDT SAM-based SARS-CoV gates show a dramatic increase of the average Rct values after storage (2600% increase after 3-day storage, 6400% increase after 7-day storage, respectively). Further, fabrication is easier and cheaper for three reasons: (1) three-step immobilization is reduced to two steps, (2) the remaining two steps are performed in aqueous solution thus avoiding use of organic solvents, (3) the peptide used for immobilization is now only composed of natural amino acids without any chemical modification (although versions of this peptide with unnatural linkers or modifications could easily be imagined). The sensors using cys-peptide coupling again selectively detect SARS-CoV-2 spike protein with a nominal LOD of 6×10⁻²² M (compare to sensors using Chem-SAM which show limit of detection of 2.8*10⁻¹⁶ M or 1.8×10⁻²⁰ M of target molecule depending on the channel material, for example SARS-CoV-2-Spike protein, in buffer solution), and achieve 10 orders of magnitude dynamic range in unprocessed saliva. The sensors fabricated with this strategy were also validated with SARS-CoV-2 pseudotyped lentivirus spiked into unprocessed human saliva and reliably detected about 20 copies of the virus particles in 5 μL sample solution.

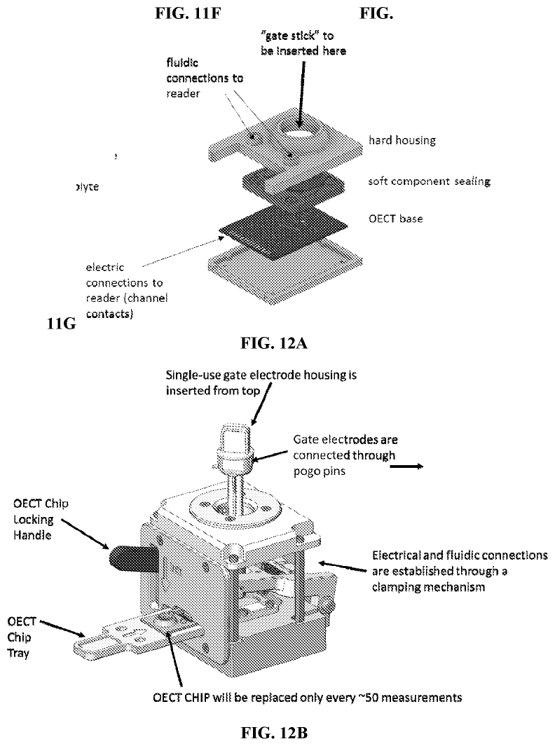

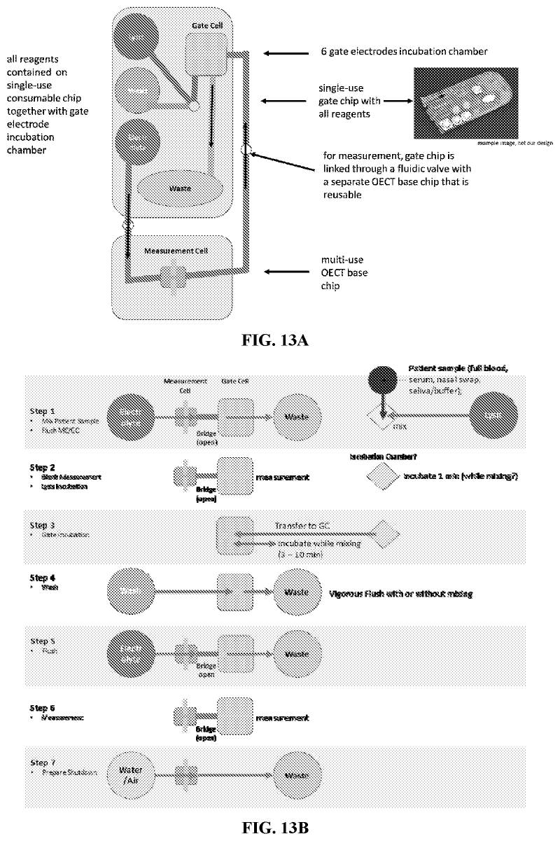

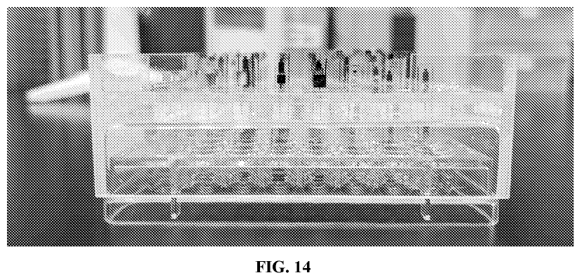

The devices can be used with minute amount of solution (as little as 5 μl) from easily accessible samples including saliva (but also blood from a finger prick); they are accessible and affordable, allowing large-scale application; easy-to-use by minimally trained users or even by the patients themselves (see, for example, the reusable device and point-of-care device shown in FIGS. 13A-13B and FIGS. 14A-14B, respectively); can be operated with an inexpensive, portable, and hand-held readout unit (for example connected to a smartphone); can be designed to detect different targets using the same platform by modular exchange of the biological detection unit (see, for example, the high-throughput device shown in FIG. 14). The device can have multiple channels in an area underneath the gate electrode, meaning that one sensing gate electrode can be used to get multiple readouts simultaneously (see, for example, the device shown in FIG. 12A), improving the sensor accuracy. These channels can be made of different organic materials.

Conventional biosensors rely on the passive diffusion-dominated transport of the target species on the sensor surface. The sample that contains the analyte to be detected is incubated with the sensor for a certain time to interact with the binding site on the sensor surface. This incubation time, sometimes up to several hours, is a significant step limiting the sensor speed.

An ACET-enhanced, OECT-based immunosensor for rapid and reliable detection of biomarkers, exemplified herein using SARS-CoV-2, is provided. The ACET can be induced by applying an AC potential to a conductive layer that is placed in proximity to the gate electrode, which allows target molecules (such as Spike protein) to accumulate on the immobilized binding partners (such as nanobodies) for the target molecules. The AC potential can be in a range from 1 Vpp to 8 Vpp or from 2 Vpp to 6 Vpp, such as about 6 Vpp (pp refers to peak to peak). The ACET-enhanced, OECT-based immunosensors can use directly functionalized OECT or indirectly functionalized OECT. The disclosed devices are based at least on the discovery (through numerical simulations and experimental studies) that ACET-induced mixing could significantly reduce the time for immunocomplex formation (≤2 min from sample to results) and can achieve a higher specificity and lower background due to electrothermal flow-induced removal of nonspecific species from the sensor surface, compared to the same OECT-based immunosensor without ACET. The ACET-enhanced, OECT-based immunosensors also show a low energy consumption (i.e., nW level, such as about 100 nW). For example, the ACET-enhanced, OECT-based immunosensors can be operated using only 100 nW power and about 2 min of incubation with a 10 μL or 5 μL sample for detecting target analytes in complex media. Two types of (semi) conducting polymers were exemplified, the p-type (p(g0T2-g6T2)) and n-type (p(C6NDI-T)), in the channel. In some embodiments, the n-type OECT can outperform the p-type OECT in terms of sensitivity and lower power consumption. The developed sensor is largely reusable, easy to manufacture, and highly modular. Its performance was validated using clinical unprocessed saliva samples from patients with COVID-19 and demonstrated sensitivity comparable to RT-PCR methods.

### I. Definitions

“Affinity interactions” as used herein refers to the combination of non-covalent interactions between a ligand and its binding partner to form a complex.

“Affinity tags” as used herein are peptide sequences appended to proteins so that they can be purified from a crude biological source using an affinity technique.

“Covalent linkage”, refers to a bond or organic moiety that covalently links molecules (e.g. fusion proteins) to a non-cellular surface.

As used herein, the term “polypeptide” refers to a chain of amino acids of any length, regardless of modification (e.g., phosphorylation or glycosylation).

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences

As used herein, an “expression control sequence” is a DNA sequence that controls and regulates the transcription and/or translation of another DNA sequence.

As used herein, “operably linked” means incorporated into a genetic construct so that expression control sequences effectively control expression of a coding sequence of interest.

As used herein, “conservative” amino acid substitutions are substitutions wherein the substituted amino acid has similar structural or chemical properties. As used herein, “non-conservative” amino acid substitutions are those in which the charge, hydrophobicity, or bulk of the substituted amino acid is significantly altered.

As used herein, the term “host cell” refers to prokaryotic and eukaryotic cells into which a recombinant expression vector can be introduced.

As used herein, “transformed” and “transfected” encompass the introduction of a nucleic acid (e.g., a vector) into a cell by a number of techniques known in the art.

### II. Compositions and Devices

### A. Directly Functionalized OECT

The disclosed devices include a directly functionalized OECT engineered to include a biorecognition layer, which includes a biorecognition element. The biorecognition element is the component that can specifically interact with its cognate target. The OECT is an electrolyte gated transistor, that is, a three-terminal electronic device which includes a source electrode, a drain electrode, a channel, and a gate electrode. In some preferred embodiments, the biorecognition layer is integrated on the gate electrode of the OECT.

### 1. Biorecognition Layer

The biorecognition layer is designed to provide a stable complex, preferably, an immunocomplex between a biorecognition element in the biorecognition layer, and the analyte/antigen of interest. The biorecognition element is preferably, not a multidomain antibody. The biorecognition layer is preferably integrated on the surface of the gate electrode of the OECT.

The biorecognition layer includes one self-assembled monolayer formed through a specific biological autocatalytic coupling strategy (herein, Bio-SAM), as disclosed herein. The Bio-SAM includes biorecognition element, such as a nanobody, a fusion protein, or a Cas protein, preferably, a nanobody.

The organization of the biorecognition element on the OECT surface can be represented by the general formula:

L1-AP1:AP2-L2-B Formula I

Where L1 is a first linker, AP1 is the first peptide binding partner harboring a specific biological recognition sequence; AP2 is the second peptide partner recognizing this sequence, i.e., AP1 and AP2 are binding partners, preferably, covalent binding partners; however, they can be members of an affinity pair; L2 is a second linker and B is the biorecognition element for the biosensor. AP2-L2-B can be produced as a single fusion protein without the need for chemical modification. The OECT surface is modified with AP1 via L1, resulting in a layer of first peptide binding partner modified OECT and provides a specific (biologically derived) recognition sequence AP1 for AP2 rather than an unspecific reactive group. Binding of AP1 and AP2 results in a biologically self-assembled monolayer, herein, Bio-SAM. Thus, the biorecognition layer includes L1 and Bio-SAM.

L1 and L2 are preferably peptide linkers sequences which are at least 2 amino acids in length. Preferably the peptide or polypeptide domains are flexible peptides or polypeptides. A “flexible linker” herein refers to a peptide or polypeptide containing two or more amino acid residues joined by peptide bond(s) that provides increased rotational freedom for two polypeptides linked thereby than the two linked polypeptides would have in the absence of the flexible linker. Exemplary flexible peptides/polypeptides include, but are not limited to, the amino acid sequences Gly-Ser, Gly-Ser-Gly-Ser (SEQ ID NO:9), Ala-Ser, Gly-Gly-Gly-Ser (SEQ ID NO: 10), (Gly4-Ser)3 (SEQ ID NO:11), and (Gly4-Ser)4 (SEQ ID NO:12), GSGSGSGS (SEQ ID NO:13) and SGSG (SEQ ID NO: 14). Additional flexible peptide/polypeptide sequences are well known in the art. In a preferred embodiment, L1 is flexible peptide as disclosed herein, modified to include a cysteine residue at its N- or C-terminus, for example, CGGSGSGSG (SEQ ID NO:22) or GSGC (SEQ ID NO:23) are preferred sequences for L1. SpyTag peptide already contains a disordered sequence at its C-terminal. If the spyTag sequence is added to the N-term of the peptide 23 (GSGC), then the overall flexible linker region can be about as long as in the case of SEQ 22.

The nanobody can be a naturally derived from immunization of an animal or synthetically-derived (i.e., a sybody) or a combination thereof. Sybodies are disclosed for example, in Walter, et al., doi: https://doi.org/10.1101/2020.04.16.045419. In some preferred embodiments, the organic molecules forming the SAM include thiols. In some preferred embodiments, the linker is a biomolecular linker formed by the bioconjugation of two peptides, such as a SpyTag/SpyCatcher bioconjugation (Zakeri, et al., Proc. Natl. Acad. Sci., 109:E690-E697 (2012)).

In a particularly preferred embodiment, the biorecognition layer does not include a SAM formed from an alkanethiol or derivative thereof (e.g. 1,6-hexanedithiol) between the OECT and L1, i.e., L1 is directly coupled to the OECT (i.e., directly functionalized OECT). The electrode surface that contains the biorecognition layer formed thereon does not contain free organic molecules, i.e., organic molecules self-assembled on the electrode surface but are not attached to L1-AP1 and thus not attached to a biorecognition element. For example, the gate electrode modified with the biorecognition layer does not contain free cysteines that are not linked to L1-AP1, such as a peptide sequence with SpyTag. Additionally, the biorecognition layer preferably includes a SpyTag/SpyCatcher bioconjugation as the linker, which allows for controllable orientation of the nanobody functionalization, maximizes the capture density of nanobodies within a small sensor area (0.64 mm²) and enhances the sensitivity of the OECT sensor. For example, at least 50-120×10¹² SpyTag peptides per cm² (such as at least 50, at least 60, at least 70, at least 80, at least and up to 90, at least 95 or about 97×10¹² SpyTag peptides) and at least 10×10¹² nanobody-SpyCatcher molecules per cm² (such as at least 10, at least 12, at least 14 and up to 15×10¹² nanobody-SpyCatcher molecules) are coupled per cm² on the surface of the gate electrode of the OECT.

i. First Binding Partner Modified OECT

The first peptide binding partner is coupled to the OECT gate electrode typically by reacting the first binding partner linked to a linker, with the gate electrode surface of the OECT under conditions resulting coupling of the linker to the surface of the OECT, following which the first peptide binding partner is bioconjugated with the second peptide binding partner, by contacting the first building partner modified OECT with a second binding partner required for formation of the SAM, i.e., the Bio-SAM. The peptide modified OECT as disclosed herein does not include free organic molecules, i.e., organic molecules self-assembled on the electrode surface but are not attached to the first linker and first peptide binding partner (L1-AP1). For example, the gate electrode of the OECT modified with the peptide does not contain free cysteines that are not linked to the first peptide binding partner.

Exemplary thiols that are excluded from coupling to the surface of the gate electrode of the OECT include, but are not limited to, alkane monothiols (e.g. methanethiol, ethanethiol, 2-propanethiol, butanethiol, pentanethiol, ter-butyl mercaptan, 1-hexanethiol, 1-octanethiol, 1-nonanethiol, 1-decanethiol, 1-undecanethiol, 1 dodecanethiol, 1-tridecanethiol, 1-tetradecanethiol, 1-pentadecanethiol, 1-hexadecanethiol, 1-octadecanethiol, 1-nonadecanethiol, and 1-icosanethiol, etc.), alkane dithiols (e.g. 1,2-ethanedithiol, 1,4-butanedithiol, 1,3-butanedithiol, 1,5-pentanethiol, 1,6-hexanedithiol (HDT), 1,7-octanedithiol, 1,8-nonanedithiol, 1,3-propanedithiol (PDT), etc.), 3-mercaptopropionic acid (MPA), thiophenol, dimercaptosuccinic acid, glutathione, 2-mercaptoethanol, lipoic acid, and 1,4-benzenedimethanethiol. Additional exemplary thiols that can form a SAM on the surface of the gate electrode of the OECT and are excluded from the gate electrode are disclosed in Love, et al., Chem. Rev., 105:1103-1169 (2005).

In a particularly preferred embodiment, the first peptide binding partner is coupled to the gate electrode surface of the OECT via a linker, preferably a peptide linker including a cysteine residue perferably at the C- or N-terminal of the peptide linker.

Referring to Formula I:

L1-AP1:AP2-L2-B Formula I

The first peptide binding partner modified surface is formed by L1-AP1, where L1 is a first linker and is coupled to the surface of the OECT gate electrode preferably via a terminal cysteine, AP1 is the first peptide binding partner; AP2 is the second peptide binding partner; AP1 and AP2 are binding partners, preferably, covalent binding partners; however, they can be members of an affinity pair; L2 is a second linker and B is the biorecognition element, such as a nanobody, a fusion protein, or a Cas protein.

ii. Bio-SAM

The biorecognition layer integrated on the gate electrode of the OECT includes one SAM, which is a biologically self-assembled monolayer formed as a result of covalent binding of two binding partners or affinity interactions of an affinity pair, thereby forming a Bio-SAM, which includes the biorecognition element, such as a protein which is not a whole antibody or fragment thereof, fusion protein, which does not include a whole antibody or fragment thereof, or a CRISPR protein (e.g., a ribonucleoprotein complex Cas:gRNA). In one preferred embodiment, the biorecognition element is a fusion protein as described herein. In another preferred embodiment, the biorecognition element is a CRISPR protein as described herein, such as a ribonucleoprotein complex Cas:gRNA.

Referring to Formula I:

L1-AP1:AP2-L2-B Formula I

Bio-SAM is formed by interaction of the first peptide binding partner AP1 with the second peptide binding partner, AP2, resulting in the formation of a SAM, the Bio-SAM, which includes the biorecognition element B, exposed for interaction with its binding partner in a sample with which it is contacted.

AP1 and AP2 in a particularly preferred embodiment include the spyTag/spyCatcher pair, with AP1 is preferably, spyTag in some preferred embodiments, for example, AHIVMVDAYKPTK (SEQ ID NO: 6), i.e., spyTag, and in preferred embodiments the spy Tag consists of SEQ ID NO: 6; VPTIVMVDAYKRYK (SEQ ID NO:7) i.e., spytag002 (Keeble, et al., Angew. Chem. Int. Ed. Engl. 2017, 56, 16521-16525) or RG VPH IVMVDAYK RYK (SEQ ID NO:8), i.e., spyTag 0003 (Keeble, et al: Proc Natl Acad Sci USA. 2019 Dec. 26; 116(52): 26523-26533).

A preferred SpyTag/SpyCatcher bioconjugation system is disclosed in Zakeri, et al., Proc. Natl. Acad. Sci., 109:E690-E697 (2012). It is based on a modified domain from a Streptococcus pyogenes surface protein (SpyCatcher), which recognizes a cognate 13-amino-acid peptide (SpyTag). Upon recognition, the two form a covalent isopeptide bond between the side chains of a lysine in SpyCatcher and an aspartate in SpyTag. The SpyTag/SpyCatcher bioconjugation is a robust method for conjugating recombinant proteins where the peptide Spy Tag can spontaneously react with the protein SpyCatcher in a facile manner and with high specificity (Zakeri, et al., Proc. Natl. Acad. Sci., 109:E690-E697 (2012); Keeble, et al., Angew. Chem. Int. Ed. Engl. 2017, 56, 16521-16525. The peptide Spy Tag (13 amino acids) spontaneously reacts with the protein SpyCatcher (12.3 kDa) to form an intermolecular isopeptide bond between the pair. The SpyTag peptide is preferably not chemically functionalized with a maleimide functional group and the SpyCatcher is linked to the biorecognition elements (e.g., nanobodies), such that upon bioconjugation between the SpyTag and the SpyCatcher, the biorecognition elements are integrated on the gate electrode surface in a uniform orientation. In optional embodiments, the SpyTag may include a linker.

Other binding partners can be used as AP1:AP2, for example, snoop Tag peptide:SnoopCatcher, (Veggiani, et al., PNAS 2016 113 (5) 1202-1207) MoonTag:MoonCatcher (homologue of SpyCatcher); the snapTag labelling system; the Sortase reaction which is connecting two shorter peptides; Coating the surface with a biotinylated peptide or biotin-modified hydrocarbon and then coupling a Streptavidin nanobody fusion protein can also be employed. SnoopTag, is a peptide which binds covalently to SnoopCatcher protein (KLGDIEFIKVNK) (SEWQ ID NO: 18). A second generation, SnoopTagJr, has been developed to bind to either SnoopCatcher or DogTag (mediated by SnoopLigase) (KLGSIEFIKVNK) (SEQ ID NO: 19). DogTag is, a peptide which covalently binds to SnoopTagJr, mediated by SnoopLigase (DIPATYEFTDGKHYITNEPIPPK) (SEQ ID NO: 20); SdyTag is, a peptide which binds covalently to SdyCatcher protein (DPIVMIDNDKPIT) (SEQ ID NO: 21) SdyTag/SdyCatcher has a kinetic-dependent cross-reactivity with Spy Tag/SpyCatcher. These systems are known in the art (reviewed Hatlem, et al., Int J Mol Sci. 2019 May; 20(9): 2129)). NAP- and CLIP-tag protein labeling systems enable the specific, covalent attachment of virtually any molecule to a protein of interest. There are two steps to using this system: cloning and expression of the protein of interest as a SNAP-tag® fusion (such as a nanobody-SNAP-tag® fusion) and labeling of the fusion with the SNAP-tag substrate of choice. The SNAP-tag is a small protein based on human 06-alkylguanine-DNA-alkyltransferase (hAGT), a DNA repair protein.

SNAP-tag substrates are dyes, fluorophores, biotin, or beads conjugated to guanine or chloropyrimidine leaving groups via a benzyl linker. In the labeling reaction, the substituted benzyl group of the substrate is covalently attached to the SNAP-tag. CLIP-tag™ is a modified version of SNAP-tag, engineered to react with benzylcytosine rather than benzylguanine derivatives. When used in conjunction with SNAP-tag, CLIP-tag enables the orthogonal and complementary labeling of two proteins simultaneously in the same cells.

a. Non-Antibody Protein Biorecognition Element

Preferred biorecognition elements (B) for incorporation into the disclosed devices are not whole antibodies, but more compact recognition domains such as a nanobody or a sybody. As used herein, non-antibody protein refers to a protein that is not a whole antibody which is a multidomain protein. By contrast, a nanobody, also known as a single domain antibody, is an antibody fragment consisting of a single monomeric variable antibody domain. Like a whole antibody, it can bind selectively to a specific antigen. With a molecular weight of only 12-15 kDa, single-domain antibodies are much smaller than common antibodies (150-160 kDa) which are composed of two heavy protein chains and two light chains and are also smaller than Fab fragments (˜50 kDa, one light chain and half a heavy chain) or single-chain variable fragments (scFv, ˜25 kDa fusion of two variable domains, one from a light and one from a heavy chain).

Camelids naturally produce one class of antibodies composed only of heavy chains in which the target recognition module is composed of a single variable domain (VHH or Nb). (De Meyer, et al., 2014; Kubala, et al., 2010). The nanobody offers unique properties, simpler structural conformation and high stability in a range of different conditions (De Meyer, et al., 2014).

In one preferred embodiment, the devices include a nanobody, which binds to an antigen from the SAR-CoV-2, such as the spike protein. Specific nanobodies against SARS-CoV-2 Spike protein (S protein) or, more specifically, against the Receptor Binding Domain (RBD) within the S1 subunit of the protein are available (Wrapp, et al., Science 2020, 367 (6483), 1260-1263). Sybodies are disclosed for example, in Walter, et al., doi: https://doi.org/10.1101/2020.04.16.045419. Examples include the SARS 1/2 nanobody (VHH72), shown below:

(SEQ ID NO: 17)
QVQLQESGGGLVQAGGSLRLSCAASGRTFSEYAMGWFRQAPGK
EREFVATISWSGGSTYYTDSVKGRFTISRDNAKNTVYLQMNSLKPDDTA
VYYCAAAGLGTVVSEWDYDYDYWGQGTQVTVSSGS

b. Fusion Proteins

In some embodiments, the biorecognition elements are expressed as fusion proteins that contain a first polypeptide domain, a linker domain and a purification tag. Biorecognition elements such as nanobodies, sybodies, etc. can readily be expressed in various formats by fusion to other proteins, peptides or effector domains, thereby tailoring their utility. Taking advantage of this feature, the disclosed biorecognition element are expressed as fusion proteins, which include a linker and preferably, a protein purification tag; and can be represented by the following general Formula II:

AP1:AP2-L2-B—C-PT Formula II,
where AP1:AP2 and L2 are as defined above for Formula I, and PT is a purification tag (an amino acid sequence that allows the polypeptides to be captured onto an affinity matrix) and C is an optional cleavage site, for example, the 3C cleavage site. In one embodiment, the biorecognition element B is oriented first in the fusion protein sequence (i.e., N-terminal). However, the biorecognition element B can be at the C-terminal end of the fusion protein. For example, a tag such as c-myc, hemagglutinin, polyhistidine, or Flag™ (Kodak) can be used to aid polypeptide purification. Such tags can be inserted anywhere within the polypeptide, including at either the carboxyl or amino terminus. The polyhistidine affinity tag, also known as the His-tag or His6, usually consists of six consecutive histidine residues, but can vary in length from two to ten histidine residues; glutathione S-transferase (GST); Maltose binding protein (MBP), calmodulin binding peptide (CBP); the intein-chitin binding domain (intein-CBD), the streptavidin tag, etc. are other known tags.

One preferred fusion protein is MTGQVQLQESGGGLVQAGGSLRLSCAASGRTFSEYAMGWFRQAPGKE REFVATISWSGGSTYYTDSVKGRFTISRDNAKNTVYLQMNSLKPDDTAV YYCAAAGLGTVVSEWDYDYDYWGQGTQVTVSSGSGSGSGSGSVDTLS GLSSEQGQSGDMTIEEDSATHIKFSKRDEDGKELAGATMELRDSSGKTIS TWISDGQVKDFYLYPGKYTFVETAAPDGYEVATAITFTVNEQGQVTVN GKATKGDAHISGLEVLFQGPTGHHHHHHHH (SEQ ID NO: 15) where the underlined residues constitute a linker (8 amino acids long), between the SAR1/2-nanobody and SpyCatcher-3C cleavage site-His8. His8 is shown in bold font and the HRV (Human rhinovirus) 3C protease cleavage site is within the underlined sequence. HRV 3C Protease is a recombinant 3C protease derived from human Rhinovirus type 14 expressed in E. coli. The enzyme has the same activity as the native protein and cleaves a specific amino acid sequence (LEVLFQ↓GP) (SEQ ID NO:16).

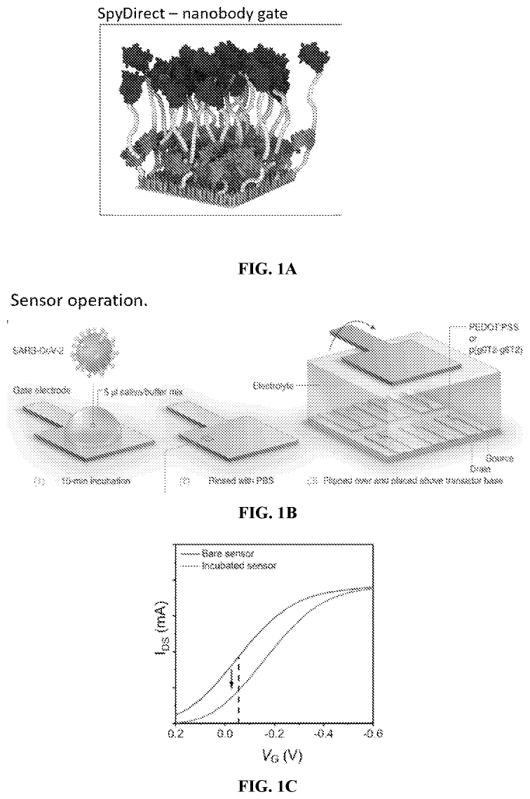

A schematic of the SpyDirect nanobody functionalized OECT sensor is shown in FIG. 1A (direct biofunctionalization of the gate electrode with a spyTag-linked cysteine peptide and a spyCatcher-fused nanobody). BSA is used as a blocking agent. The sensor is incubated with a saliva-buffer mixture. (FIG. 1B) The bare sensor gate or the incubated sensor gate is mounted on the top of the channel for signal acquisition. As exemplified therein, a synthetic SpyTag peptide is directly coupled to the gold surface via a linker, without the need for the 1,6-hexanedithiol (HDT) monolayer to form a Chem-SAM as disclosed for example, in PCT/IB2021/055981. The nanobody-SpyCatcher fusion protein then attaches itself to the spyTag through the autocatalytic formation of a covalent SpyCatcher-Spy Tag bond, forming the Bio-SAM. The nanobody domain defines sensor specificity and is interchangeable (examples below demonstrate the device design and its function with GFP, SARS-CoV, and MERS-CoV).

In some embodiments, the peptide binding partners are prepared by chemical synthesis. The peptide binding partners can be chemically synthesized by methods known in the art, such as by using solid phase peptide synthesis, solution phase synthesis, chemical ligation (see e.g., Chandrudu, et al., Molecules, 18 (4): 4373-4388 (2013)). In some embodiments, the SpyTag can be chemically synthesized. For example, the SpyTag, which includes a linker and optionally a cleavage site and/or a purification tag, is chemically synthesized by a known method as described above, and can be represented by the general formula III below.

Alternatively, the peptide binding partners can be expressed as fusion proteins that contain a linker domain and a purification tag. Peptide binding partners such as SpyTag can readily be expressed in various formats by fusion to other proteins, peptides or effector domains, thereby tailoring their utility. In these embodiments, the disclosed Spy Tag can be expressed as fusion proteins, which include a linker and preferably, a protein purification tag; and can be represented by the following general formula III:

L1-AP1-C—PT
where AP1 and L1 are as defined above for Formula I, C and PT are as defined above for Formula II. AP1 is preferably C-terminally cysteine linked.
   c. CRISPR Protein

Clustered regularly interspaced short palindromic repeats (CRISPR) and CRISPR-associated (Cas) protein systems have been harnessed for gene editing and the nuclease-dead mutants (dCas9) have been used for gene regulation across diverse species. There are numerous classes and types of Cas effectors reported in the literature. For example, it is possible to select Cas effectors that selectively bind or cleave DNA, RNA, or both. Moreover, many, but not all, Cas proteins have two cleavage activities, one in cis (to the target molecule) and the other in trans (also referred to as collateral activity that cleaves “bystander” non-specific nucleic acids after a specific target has been recognized). Due to their programmability (i.e., customized design of guide RNA spacer sequences) and multiplexing features (i.e., by using Cas orthologues that target different nucleic acid molecules or cleave on different sequences) Cas-based systems are very appealing for molecular diagnostics.

In some embodiments, the CRISPR enzyme is mutated with respect to a corresponding wild-type enzyme such that the mutated CRISPR enzyme lacks the ability to cleave one or both strands of a target polynucleotide containing a target sequence. Cas proteins devoid of nucleolytic activity (dead Cas proteins; dCas) are known. Inactivation through mutation of both nuclease domains generates a catalytically dead Cas. The CRISPR enzyme can be Cas9, dCas9 (dead Cas9), dCas12, Cas12a, Cas12b, dCas13 or Cpf1, etc Catalytically inactive, or “dead,” Cas9 (dCAS9) (or any Cas enzyme) is mutated version of the protein cannot cut, but still binds tightly to a particular DNA sequence specified by the guide RNA. An example is Streptococcus pyogenes Cas9 (SpCas9) can also be used in its deactivated form (SpdCas9) i.e. deactivated SpdCas9. In one preferred embodiment, the Cas effector is from Staphylococcus aureus (SauCas9), Leptotrichia wadeii. (LwaCas13a), Leptotrichia buccalis. (LbuCas13a) or Eubacterium siraeum (EsCas13d).

iii. Blocking Agents

Optionally, the biorecognition layer includes a blocking agent. Exemplary blocking agents include, but are not limited to, bovine serum albumin (BSA), ethanolamine (ETA) and Casein. In some preferred embodiments, the blocking agent included in the biorecognition layer is BSA. While not being bound by theory, the blocking agent provided to capture potentially contaminating proteins in samples included analyte, which are contacted with the biorecognition layer for binding of the analyte to its binding partner (i.e., biorecognition element) present in the biorecognition layer.

### 2. Indirectly Functionalized OECT

Alternatively, the OECT may be functionalized using an indirect method, as described in PCT/IB2021/055981. For example, the biorecognition layer includes two self-assembled monolayers (SAMs the first of which is formed from organic molecules, chemically modified as disclosed in PCT/IB2021/055981, i.e. Chem-SAM, and the second of which is a Bio-SAM, as disclosed herein). The Bio-SAM includes biorecognition element such as a nanobody, a fusion protein, or a Cas protein described above, preferably, a nanobody.

The organization of the biorecognition layer indirectly functionalized on the OECT surface can be represented by the general formula:

N-L₁-AP₁-AP₂-L₂-B Formula I′
where N is one or more organic molecules capable of self-assembly to form a first SAM, L₁ is an optional first linker, AP₁ is the first peptide binding partner; AP₂ is the second peptide partner, L2 is a second linker, and B is the biorecognition element. The first SAM formed by N is chemically modified with AP₁, resulting in Chem-SAM. AP₁, AP₂, L2, and B can be any of those described above for directly functionalization. Thus, the biorecognition layer includes Chem-SAM and Bio-SAM. The first-SAM on the OECT gate electrode is typically formed by reacting a plurality of the organic molecules on the gate electrode surface of the OECT under conditions resulting in self-assembly, following which it chemically reacts with L1-AP1 to form Chem-SAM.

In contrast, the method for direct functionalization of the gate electrode of the OECT excludes a step of contacting the gate electrode with a solution containing organic molecules capable of self-assembly, such as cysteines, prior to contacting the gate electrode with L1-AP1, and thus eliminates the surface chemistry between the organic molecule and L1-AP1.

### 3. OECT Components

The disclosed device includes a biorecognition layer described above and its integration with a high gain, ion-to-electron transducing device, the OECT. The OECT is a three-terminal transistor which includes a source electrode, a drain electrode, a channel, and a gate electrode. The channel electronically connects the source electrode and drain electrode. The gate electrode that is integrated/functionalized with a biorecognition layer. The source electrode and the drain electrode are placed apart and connected electronically by a corresponding channel.

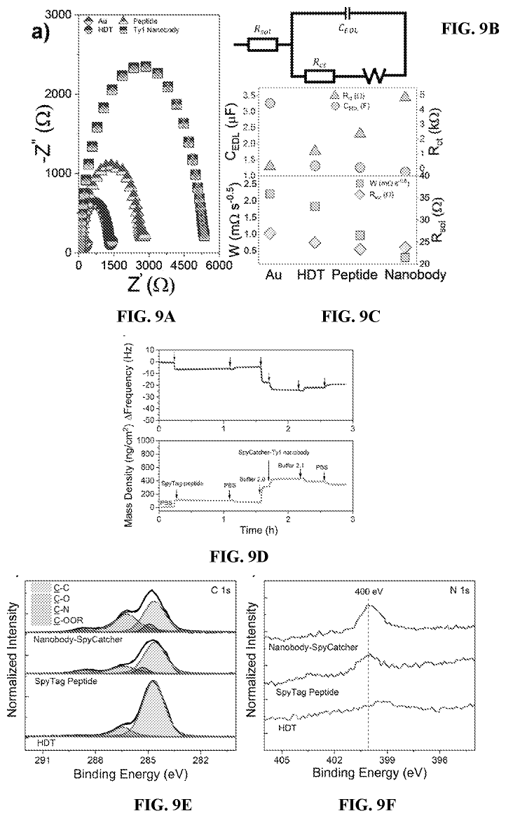

In some embodiments, the source electrode, the drain electrode, and the channel can be patterned on a supporting substrate, such as a glass substrate, a silicon substrate, or a plastic substrate, such as a polyimide substrate or a textile. For example, the source electrode, drain electrode, and channel are patterned on a glass substrate. The gate electrode is placed separately from the source electrode, the drain electrode, and the channel. The gate electrode is removable, i.e., it is not physically connected to the supporting substrate on which the source, drain, and channel are patterned. This configuration allows easy handling of the gate electrode when in use. For example, the user can incubate the gate electrode with a blank solution or biological sample and perform subsequent rinse, away from the rest of the OECT components, and then place the gate electrode in position for measurement (see, for example, FIGS. 1B and 8C).

During measurements, the channel and optionally the source electrode and drain electrode are in contact with an electrolyte solution, into which the gate electrode is also immersed. Optionally, the OECT includes a reservoir to contain the electrolyte solution. The reservoir (i.e., PDMS well) is placed on top of the channel to contain the electrolyte solution such that the channel and the gate electrode are in contact with the electrolyte solution. Optionally, the reservoir is configured such that the electrolyte solution contained therein also contact the source and drain electrodes, but are insulated with an insulator such as parylene or SU-8.

In some embodiments, the device includes more than one OECT in the form of an array. When the device contains an array of OECTs, each OECT may contain a source electrode, a drain electrode, and a corresponding channel, and all the OECTs in the array use a common gate electrode. For example, if the OECT array includes a plurality of OECTs, for example 2, 3, 4, 5 or 6 OECTs, where each OECT contains a source electrode, a drain electrode, and a corresponding channel; however, all the OECTs in the array use a common gate electrode that is integrated with a biorecognition layer. The source electrode, drain electrode, and corresponding channel of each of the OECTs are patterned on a supporting substrate. Optionally, when the device contains an array of OECTs, each OECT may contain a source electrode, a drain electrode, a corresponding channel, and a gate electrode, such that each gate electrode can be engineered with the same or different biorecognition element for detecting one or more than one analyte simultaneously. The device may be incorporated into a microfluidics configuration. Examples of device including an array of OECTs are shown in FIGS. 1B, 12A, and 14.

The channel is typically made of an ion-permeable organic electronic material, through which holes or electrons flow from the source electrode to the drain electrode. The OECT relies on ions that are injected from the electrolyte solution into the ion-permeable organic electronic material, thereby changing its doping state and thus its conductivity. The operation of OECT is controlled by voltages applied to the gate electrode (gate voltage, VG) and to the drain electrode (drain voltage, VD), which are referenced with respect to the source electrode. The drain voltage induces a current (drain current, ID), which is proportional to the quantity of mobile holes or electrons in the channel, and thus probes the doping state of the organic electronic material. The gate voltage controls the injection of ions into the channel and thus the doping state of the organic electronic material, resulting in a change in ID. The change in ID may be expressed as a normalized response (also referred herein as “NR”) which can be calculated based on equation 1:

NR=(|ID−I0|)/I0 equation 1
where I0 is the drain current obtained after incubation of the OECT with a blank solution (i.e., a solution in the absence of the target analytes) under the same measurement conditions as the biological sample.

The doping changes in OECT occur over the entire volume of the channel because of the injection barrier-free penetration of electrolyte ions into the bulk of the organic channel, causing a large modulation of the carrier density therein. The device translates small ionic fluxes in the electrolyte into a large electrical readout from the channel. Therefore, the transducing event is coupled with amplification, and endows the OECT with high gain at low voltages (<1V).

i. Source and Drain Electrodes

The source and drain electrodes are made from materials capable of conducting an electric current. The electrode materials can be organic or inorganic in nature, as long as it is able to conduct electrons and inject electronic charges into the channel material. The electrodes can be a polymeric electrode, a metallic electrode, a carbon-based material, a metal oxide, or a modified electrode. In some embodiments, the source and drain electrodes are made from an electrochemically inert material such as gold, platinum, chromium, or a conductive form of carbon, or a combination thereof. In some embodiments, the source and drain electrodes are gold electrodes. In some embodiments, the source and drain electrodes are a stack of metal layers, such as gold layer coated on a chromium layer. Each of the source and drain electrodes may be insulated using an insulator such as parylene. In some embodiments, SU-8 (epoxy based photoresist used to pattern electronics with photolithography (also used as an insulator)) is used to insulate the source and drain electrodes.

In some embodiments, the electrodes are made from a metallic conductor. Suitable metallic conductors include but are not limited to gold, chromium, platinum, iron, nickel, copper, silver, stainless steel, mercury, tungsten and other metals suitable for electrode construction. The metallic conductor can be a metal alloy which is made of a combination of metals disclosed above, such as gold/chromium. In some embodiments, the electrode can be formed from different layers of metals, such as a gold layer coated on a chromium layer. In addition, conductive substrates which are metallic conductors can be constructed of nanomaterials made of gold, cobalt, diamond, and other suitable metals.

In other embodiments, the electrodes are made from carbon-based materials. Exemplary carbon-based materials are conducting polymers (in the form of films or fibers) carbon cloth, carbon paper, carbon screen printed electrodes, carbon paper, carbon black, carbon powder, carbon fiber, singe-walled carbon nanotubes, double-walled carbon nanotubes, multi-walled carbon nanotubes, carbon nanotube arrays, diamond-coated conductors, glassy carbon and mesoporous carbon. In addition, other exemplary carbon-based materials are graphene, graphite, uncompressed graphite worms, delaminated purified flake graphite, high performance graphite and carbon powders, highly ordered pyrolytic graphite, pyrolytic graphite, and polycrystalline graphite.

The electrodes can be doped semiconductors. Suitable semiconductors are prepared from silicon and germanium, which can be doped (i.e., the intentional introduction of impurities into an intrinsic semiconductor for the purpose of modulating its electrical and structural properties) with other elements. The semiconductors can be doped with phosphorus, boron, gallium, arsenic, indium or antimony, or a combination thereof.

Other electrode materials can be metal oxides, metal sulfides, main group compounds, and modified materials. Exemplary materials of this type are nanoporous titanium oxide, tin oxide coated glass, glass, cerium oxide particles, molybdenum sulfide, boron nitride nanotubes, aerogels modified with a conductive material such as gold, solgels modified with conductive material such as carbon, ruthenium carbon aerogels, and mesoporous silicas modified with a conductive material such as gold.

The source and drain electrodes can be any shape appropriate such as cuboid, cubic, circular, and cylindrical. In some preferred embodiments, the electrodes are cuboid gold electrodes or cuboid gold coated chromium electrodes. In some embodiments, each of the source and drain electrodes has a first dimension (i.e., width), a second dimension (i.e. length), and a third dimension (i.e. thickness). For example, each of the source and drain electrodes has a width in a range from 100 μm to 1 mm (e.g., 0.8 mm), a length in a range from 100 μm to 1 mm (e.g., 0.8 mm), and a thickness of about 100 nm.

ii. Channel

Generally, the channel is configured to establish the electrochemical connection between a pair of sources and drain electrodes such that holes or electrons flow from the source electrode to the drain electrode.

The channel typically contains an ion-permeable organic electronic material, such as a conducting polymer disclosed in, for example, Rivnay, et al., Nature Reviews, 3:17086 (2018) and Sun, et al., J. Mater. Chem. C, 6:11778-11784 (2018). The channel of the OECT may be formed directly from a conducting polymer or by incorporating a conducting polymer on the surface of a conductive substrate. For example, the conducing polymer can be spin coated, drop casted, inkjet printed, or screen printed on the surface of an conductive substrate, such as a gold substrate or a gold coated chromium substrate. Any suitable conducting polymers can be used for the channel of the OECT. In some embodiments, the conducting polymer for the channel of the OECT is a p-type polymer. In some embodiments, the conducting polymer for channel of the OECT is an n-type polymer. Depending on channel material, the OECT can operate in depletion or in accumulation mode.

An exemplary conducting polymer for the channel is poly(3,4-ethylenedioxythiophene) doped with poly(styrene sulfonate) (PEDOT:PSS). The conducting PEDOT is p-type doped (oxidized), which leads to mobile holes that can hop from one chain to another, resulting in a hole current once a drain voltage is applied. These holes are compensated by the sulfonate anions of PSS. Channels made of PEDOT:PSS can work as depletion mode OECTs. For example, in the absence of a gate voltage, a hole current flows in the channel. Once a positive gate voltage is applied, cations from the electrolyte are injected into the channel and the anions are compensated, resulting in a decrease in the number of holes in the channel. This results in a decrease in the drain current. Alternatively, the channel can be made of materials that work in accumulation mode OECT, such as a semiconductor based on a polythiophene with a sulfonate group attached to the backbone with a hexyl chain (PTHS) (Inal, et al., Adv. Mater., 26:7450-7455 (2014)), an ethylene glycol unit attached to bithiophenes (Moser, et al., Adv. Mater., 32:2002748 (2020)) or a g7-NDI-Br and NDI-T2 copolymer (P-90) (Giovannitti, et al., Chem. Mater., 30:2945-2953 (2018)). In accumulation mode p-type OECT, application of a negative gate voltage causes injection of anions into the channel and a corresponding accumulation of holes, leading to an increase in the drain current ID. One can also use n-type semiconductors and build accumulation mode n-type OECTs. In accumulation mode n-type OECT, application of a positive gate voltage causes injection of cations into the channel and a corresponding accumulation of electrons, leading to an increase in the drain current ID.

Other suitable conducting polymers for the channel include, but are not limited to, conductor based on PEDOT with a pendant sulfonate group (PEDOT-S), PEDOT doped with tosylate (PEDOT: TOS), PEDOTOH:ClO4, PEDOT-co-PEDOTOH:ClO4 (Schmode, et al., Chem. Mater., 31 (14): 5286-5295 (2019)), poly(2-(3,3′-bis(2-(2-(2-methoxyethoxy) ethoxy) ethoxy)-[2,2′ bithiophen]-5 yl)thieno[3,2b]thiophene), p(g2T TT), poly((ethoxy)ethyl 2-(2-(2 methoxyethoxy) ethoxy)acetate)-naphthalene 1,4,5,8 tetracarboxylic-diimide-co 3,3′ bis(2-(2-(2 methoxyethoxy)ethoxy)ethoxy)-(bithiophene)) p(gNDI g2T), P3HT (poly(3-hexylthiophene-2,5-diyl)), BBL (polybenzimidazo-benzoisoquioline), PTHS-TMA+-co-P3HT (poly[(6-thiophen-3-yl) hexane-1-sulfonate-co-3-(hexylthiophene)]), poly(N,N′-bis(7-glycol)-naphthalene-1,4,5,8-bis(dicarboximide)-co-2,2′-bithiophene-co-N,N′-bis(2-octyldodecyl)-naphthalene-1,4,5,8-bis(dicarboximide), naphthalene-1,4,5,8-tetracarboxylic-diimide-bithiophene (NDI-T2) based polymer with 90% glycol chain percentage (P-90), and glycolated Diketopyrrolopyrroles (DPPs) or glycolated thiophenes, bithiophenes such as p(g0T2-g6T2) (Moser, et al., Advanced Materials, 32:2002748 (2020)).

Examples of suitable polymers for the channel in a depletion mode OECT are PEDOT polymers or copolymers thereof, such as polymers based on PEDOT with a pendant sulfonate group (PEDOT-S), PEDOT doped with tosylate (PEDOT:TOS), PEDOTOH:ClO4, PEDOT-co-PEDOTOH:ClO4, and PEDOT:PSS.

Examples of suitable polymers for the channel in an accumulation mode OECT are poly(2-(3,3′-bis(2-(2-(2-methoxyethoxy) ethoxy)ethoxy)-[2,2′ bithiophen]-5 yl)thieno[3,2 b] thiophene), p(g2T TT), poly((ethoxy)ethyl 2-(2-(2 methoxyethoxy) ethoxy)acetate)-naphthalene 1,4,5,8 tetracarboxylic-diimide-co 3,3′ bis(2-(2-(2 methoxyethoxy)ethoxy) ethoxy)-(bithiophene)) p(gNDI g2T), P3HT (poly(3-hexylthiophene-2,5-diyl)), BBL (polybenzimidazo-benzoisoquioline), PTHS-TMA+-co-P3HT (poly[(6-thiophen-3-yl) hexane-1-sulfonate-co-3-(hexylthiophene)]), poly(N,N′-bis(7-glycol)-naphthalene-1,4,5,8-bis(dicarboximide)-co-2,2′-bithiophene-co-N,N′-bis(2-octyldodecyl)-naphthalene-1,4,5,8-bis(dicarboximide), naphthalene-1,4,5,8-tetracarboxylic-diimide-bithiophene (NDI-T2) based polymer with 90% glycol chain percentage (P-90), and glycolated Diketopyrrolopyrroles (DPPs) or glycolated thiophenes, bithiophenes such as p(g0T2-g6T2).

In some embodiments, the conducting polymer for the channel of the OECT is poly(3,4-ethylenedioxythiophene) doped with poly(styrene sulfonate) (PEDOT:PSS) or any other mixed (ionic and electronic) semiconductor. In some embodiments, the conducing polymer for the channel of the OECT is p(g₀T2-g6T2). In some embodiments, the conducting polymer for the channel of the OECT is p(g₃C₂T2-T). In some embodiments, the conducting polymer for the channel of the OECT is p(C6NDI-T).

Typically, the channel has a first dimension (i.e., width), a second dimension (i.e. length), and a third dimension (i.e. thickness). The length is the distance between the source and drain electrodes. Width is the remaining dimension of that rectangle The length and width of the channel can be a value between about 5 μM and about 5 mm, for example, between 5 μm and 1000 μm, between 5 μm and 100 μm, between 5 μm and 100 μm, or between 10 μm and 100 μm, for example, about 10 μm or about 100 μm. The width of the channel can be between 10 μm and 1000 μm, between 20 μm and 500 μm, or between 20 μm and 200 μm, for example, about 100 μm. The thickness of the channel can be a value between about 10 nM and about 200 nm, for example, between nm and 150 nm, between 20 nm and 100 nm, between 50 nm and 150 nm, or between 50 nm and 100 nm, for example, about 85 nm.

The first dimension and the second dimension can be the same or different. In some embodiments, the width of the channel is the same as the length of channel. For example, the channel has a width of about 100 μm and a length of about 100 μm. In some embodiments, the width of the channel is smaller than the length of the channel. In some preferred embodiments, the width of the channel is larger than the length of the channel. For example, the channel has a width of about 100 μm and a length of about 10 μm. In some embodiments, the OECT having a small channel (e.g., a width of about 100 μm and a length of about 10 μm) is more sensitive (i.e. higher NR) than the same OECT but having a larger channel (e.g. a width of about 100 μm and a length of about 100 μm) under the same measurement conditions (e.g. VG, VD, incubation time, temperature, and pressure).

Typically, the channel remains unmodified, i.e., it is not chemically modified to anchor binding partners. The analyte recognition takes place at the functionalized gate electrode, which is not in physical contact with the channel. This configuration allows prolonged shelf life of the device compared with OECT containing channels modified with binding partners. For example, the OECT disclosed herein can preserve its drain current (i.e. change of drain current is less than about 5% compared with a fresh sensor) after storing at ambient conditions (i.e. about 25° C. at 1 atm) for at least 6 months in air or for at least 1 year in nitrogen or vacuumed environment. For example, the OECT disclosed herein can preserve its NR determined with a standard solution containing the same amount of the same analyte (i.e. change of NR is less than about 5% compared with a fresh sensor) after storing at ambient conditions (i.e. about 25° C. at 1 atm) for 6 months in air or 1 year in nitrogen or vacuumed environment.

iii. Gate Electrode

The gate electrode is configured to control the injection of ions into the channel, typically placed in the measurement solution with the source electrode, the drain electrode and the channel, but not in physical contact with these components.

The gate electrode can be any shape appropriate such as cuboid, cubic, circular, and cylindrical. In some embodiments, the gate electrode is a circular electrode having a diameter in a range from 0.5 mm to 10 mm, from 1 mm to 10 mm, from 0.5 mm to 5 mm, or from 1 mm to 5 mm, such as 2.8 mm or 5 mm. In some embodiments, the gate electrode is a circular electrode having a diameter of about 5 mm. In some embodiments, the gate electrode is a square electrode having a length in a range from 0.1 mm to 10 mm, from 0.1 mm to 8 mm, from 0.1 mm to 5 mm, from 0.5 mm to 10 mm, or from 0.5 mm to 5 mm, such as about 0.8 mm or about 4.8 mm. In some embodiments, the gate electrode is a square electrode having a length of about 0.8 mm.

In some embodiments, the OECT contains a gate electrode having any shape and size as described above and a channel having any shape and size as described above. In some embodiments, the OECT contains a square gate electrode having any length as described above and a rectangular channel having any size (i.e., width and length) as described above. In some embodiments, the OECT contains a circular gate electrode having any diameter as described above and a cuboid channel having any size (i.e., width, length, and thickness) as described above. For example, the OECT contains a square gate electrode having a length of about 0.8 mm and a rectangular channel having a width of about 100 μm and a length of about 10 μm. For example, the OECT contains a circular gate electrode having a diameter of about 5 mm and a cuboid channel having a width of about 100 μm, a length of about 10 μm, and a thickness of about 85 nm.

Generally, the gate electrode can be made from a substrate coated with any conducting materials descried above for the source and the drain electrodes (e.g., Pt, Au, Cr, Au coated Cr, etc.) or a conducting polymer, such as poly(fluorine)s, polyphenylenes, polypyrenes, polyazulenes, polynaphthalenes, poly(pyrrole)s, polycarbozoles, polyindoles, polyzaepines, polyanilines, poly(thiophene)s, poly(3,4-ethylenedioxythiophene), poly(p-phenylene sulfide), poly(acetylene)s, or poly(p-phenylene vinylene). In some embodiments, the gate electrode contains two or more conducting materials. For example, the gate electrode contains two conducting materials, where the first conducting material can be a material disclosed above for the source and the drain electrodes and a second conducting material can be a conducting polymer. In some embodiments, the gate electrode contains two conducting materials, where the first conducting material is a metallic conductor of a first type and the second conducting material is a metallic conductor of a second type. The two conducting materials may be coated simultaneously or subsequently on the substrate. An exemplary gate electrode is formed from a Kapton (polyimide) substrate sputter coated with Cr and Au subsequently. Another exemplary gate electrode is formed from a glass substrate sputter coated with Cr and Au subsequently.

Typically, the gate electrode is engineered to include a biorecognition layer. For example, the gate electrode is modified sequentially with a first peptide binding partner via a first linker and a biorecognition element, such as nanobody, via a second peptide binding partner to form the biorecognition layer on the gate electrode (i.e., direct functionalization). For example, the gate electrode is modified sequentially with a thiol-containing organic molecule, a first peptide binding partner via a first linker, and a biorecognition element, such as nanobody, via a second peptide binding partner to form the biorecognition layer on the gate electrode (i.e., indirect functionalization).

iv. Electrolyte Solution

When in use, the electrolyte solution is in electrical contact with the channel and the gate electrode, and optionally the source electrode and the drain electrode (the last two are insulated with an insulator). The electrolyte solution is a solution that contains ions or molecules that have lost or gained electrons and is electrically conductive and mostly aqueous. Electrolyte solutions include, but are not limited to, buffers such as water, phosphate buffered solution, phosphate buffered saline, salt water, MES buffer, Bis-Tris buffer, ADA, ACES, PIPES, MOPSO, Bis-Tris propane, BES, MOPS, TES, HEPES, DIPSO, MOBS, TAPSO, Trizma, HEPPSO, POPSO, TEA, EPPS, Tricine, Gly-gly, Bicine, HEPBS, TAPS, AMPD, TABS, AMPSO, CHES, CAPSO, AMP, CAPS, CABS. The electrolyte solution can have a pH between about 4 and about 8.5, between about 4.5 and about 8.5, between about 5 and about 8.5, between about 5.5 and about 8.5, between about 6 and about 8, or between about 6.5 and about 7.5, preferably about 7.4.

v. Reservoir

The device may include a reservoir to contain the electrolyte solution. The reservoir may be incorporated in the OECT by any suitable methods. For example, the reservoir is glued or molded on top of the channel. The reservoir can also be a microfluidic channel. When more than one channels are included in the device as an array, the reservoir is incorporated in the device such that the electrolyte solution contained therein is in contact with all of the channels and the gate electrode, when in use.

The reservoir is typically defined by a side wall and a bottom surface, and contains an opening configured to allow the electrolyte solution to enter the reservoir. The reservoir may have any suitable shapes, such as a cylindrical well, a cubic shape, or a cuboid shape. The bottom surface is formed from at least a portion of the channel and optionally, at least a portion of the side wall is formed from the source electrode and the drain electrode, respectively. For example, the reservoir is a cylindrical well and has a bottom surface formed from at least a portion of the channel. The side wall is perpendicular to the surface of the channel and at least a portion of the side wall is in contact with the source electrode and drain electrode. The cylindrical reservoir contains an opening to allow the electrolyte solution to enter the reservoir. An exemplary OECT containing a cuboid, trapezoidal, or cylindrical reservoir to contain the electrolyte solution is shown in FIGS. 1B, 8C, and 12A.

For example, the reservoir is a cuboid defined by a bottom surface and four side walls. The bottom surface is formed by at least a portion of the channel and each of the first and second side walls are parallel to each other and vertically placed on top of each of the source electrode and drain electrode. The third and fourth side walls are parallel to each other and perpendicular to the first and second side walls, such that the reservoir contains an opening to allow the electrolyte solution to enter the reservoir.

For example, the reservoir is a cuboid and has a bottom surface formed by the channel, a first side wall formed from a portion of the source electrode, a second side wall formed from a portion of the drain electrode, and a third side wall and a fourth side wall that are parallel to each other and perpendicular to the first side wall and second side wall, such that the reservoir contains an opening to allow the electrolyte solution to enter the reservoir. When an electrolyte solution is in the reservoir, the channel, at least a portion of the source electrode and the drain electrode, and the gate electrode are in contact with the electrolyte solution.

The reservoir can be made from any suitable inert material, such as plastic, glass, or a polymeric material, such as polydimethylsiloxane (PDMS).

### B. Convection Driven Ultra-Rapid Detection Transistors

One embodiment provides a directly nanobody-functionalized organic electrochemical transistor (directly functionalized-OECT) and another embodiments provides indirectly nanobody-functionalized organic electrochemical transistor (indirectly functionalized-OECT), either, incorporated with the micro-stirring effect of alternating current electrothermal flow (ACET) for the ultra-rapid detection of single-molecule-to-nanomolar levels of a pathogen such as the severe acute respiratory syndrome coronavirus 2 (SARS-CoV-2) in complex bodily fluids.

The ACET-enhanced, OECT-based immunosensors can use directly functionalized OECT or indirectly functionalized OECT, such as any of those described above, and a conductive layer that is placed in close proximity (i.e., the shortest edge-to-edge distance of less than 1 μm, measured from an edge of the gate electrode to an edge of the conductive layer) to the gate electrode of the OECT for inducing ACET. Typically, the conductive layer is placed along an edge of the gate electrode and surrounds at least a portion of the gate electrode. Optionally, the ACET-enhanced, OECT-based immunosensor includes more than one conductive layer, where each conductive layer is placed along an edge of the gate electrode and the conductive layers, together, surround at least a portion of the gate electrode. The conductive layer can have any suitable shape, as long as it can be placed along an edge of the gate electrode and surrounds at least a portion of the gate electrode. For example, as shown in FIG. 8A, the gate electrode 10 is circular in shape, and the arch-shaped conductive layer 20 is placed along the edge 12 of the gate electrode and surrounds at least a portion of the circle 10 concentrically. Optionally, the conductive layer contains one or more extended portions (see, e.g., 22a and 22b in FIG. 8A) to facilitate application of an AC potential from a power source to the conductive layer. When in use, ACET can be induced by applying an AC potential to the conductive layer optionally through one or more extended portions. The induced ACET generates a electrohydrodynamic force on the fluid that stirs the solute molecules by the induced flow (also referred to herein as “micro-stirring effect”), allowing target molecules (such as Spike protein) to accumulate on the immobilized binding partners (such as nanobodies) for the target molecules at the gate electrode.

The conductive layer can be formed using any conductive material suitable for forming an electrode, such as any of those described above for forming the source electrode, drain electrode, channel, and gate electrode. For example, the conductive layer is formed by gold or gold coated on chromium.

The disclosed ACET enhanced, OECT-based sensors are based at least on the discovery (through numerical simulations and experimental studies) that ACET-induced mixing could significantly reduce the time for immunocomplex formation (≤2 min from sample to results) and can achieve a higher specificity and lower background due to electrothermal flow-induced removal of nonspecific species from the sensor surface, compared to the same OECT-based immunosensor without ACET. The ACET-enhanced, OECT-based immunosensors also show a low energy consumption (i.e., nW level, such as about 100 nW). For example, the ACET-enhanced, OECT-based sensors can be operated using only 100 nW power and about 2 min of incubation with a 10 μL or 5 μL sample for detecting target analytes in complex media.

The ACET flow induced by a biased AC electrical field can rapidly convect the analyte onto concentric gate electrodes within 2 minutes, such as within a minute, and SARS-CoV-2 spike proteins (S1) or receptor-binding domains (RBDs) are captured via recognition units (nanobody) while sweeping nonspecifically bound ones away from the surface.

The sensors include of a solution-processable conjugated polymer in the transistor channel and a large variety of recognition units on gate electrodes, for example, a high-density and orientation-controlled bioconjugation of nanobody-SpyCatcher fusion proteins on the gate electrode, either directly or indirectly functionalized on the gate electrode as described above. Synergetic effects of ACET and OECT provide results after 2 min or 1 min of exposure to 10 μL or 5 μL of a sample, maintaining high specificity and single-molecule sensitivity in biological samples, such as human saliva, with low power consumption, such as ≤100 W.

### II. Methods Od Making

The disclosed biosensors integrate OECT technology engineered to include a biorecognition layer by functionalizing the sensing electrode (i.e., gate electrode), via a binding partner, preferably, covalent binding partners or an affinity pair (e.g. the SpyTag/SpyCatcher conjugation system), and a biorecognition element. Optionally, the biorecognition layer is also exposed to a blocking agent, which can be introduced simultaneously with the biorecognition element or subsequently to the biorecognition element.

Methods of making an OECT or an array of two or more OECTs are known. An exemplary method of making an OECT array is described in the Examples. The biorecognition elements, such as nanobody units, can be immobilized with uniform orientations using the methods described herein; this allows the biorecognition layer to be more precise and compact.

Generally, methods of integrating a biorecognition layer on an electrode, such as a gate electrode of an OECT, include: (i) contacting at least a portion of the surface of the electrode with a first solution containing a peptide sequence which preferably includes a cysteine residue at its N- or C-terminus, a peptide linker and a first peptide binding partner, to produce a first peptide binding partner-modified OECT surface, and (ii) contacting first peptide binding partner-modified surface with a second solution containing a second peptide (which is a binding partner to the first peptide), a biorecognition element for the analyte of interest and optionally, a blocking agent, where the first peptide conjugates with the second peptide to form a monolayer, referred to herein as Bio-SAM. The method for functionalizing the gate electrode of the OECT excludes a step of contacting at least a portion of the surface of the gate electrode with a solution containing thiol-containing organic molecules, such as cysteines, prior to step (i), to form a Chem SAM. That is, the method for electrode functionalization disclosed herein does not form a Chem SAM and then chemically attach the first linker and first peptide binding partner to the Chem SAM, which would result in free organic molecules on the gold surface (i.e., organic molecules in the Chem SAM that are not attached to a first peptide binding partner and thus not attached to a biorecognition element).

The OECT electrode modified by Bio-SAM results in a modified OECT gate electrode surface, with the recognition element exposed for interaction with an analyte (to which it binds) from a sample. (See FIGS. 1A and 1B). The second solution preferably includes a blocker molecule such as BSA with, or without a mild detergent for example, Tween®20 (Polyethylene glycol sorbitan monolaurate).

Referring to Formula I, (i) the surface of the electrode is contacted with a composition including AP₁ and L₁ under conditions that result in chemical coupling of the AP₁ to the surface of the OECT via L₁, thus forming an AP₁-L₁-OECT electrode surface, and (ii) contacting the AP₁-L₁-OECT electrode surface with a composition including AP₂-L₂-B— under conditions resulting in conjugation of AP₁ and AP₂ to form a biologically self-assembled monolayer, herein Bio-SAM. (FIG. 1A). The composition in step (ii) preferably includes a blocker molecule such as BSA with, or without a mild detergent for example, Tween®20 (Polyethylene glycol sorbitan monolaurate). When spy Tag and Spycatcher are used as binding partners, spyTag is AP1 and spyCatcher is Ap2 i.e., the surface of the OECT is contacted with the solution containing spy Tag first. This is in contrast to the procedure described in Oloketuyi, et al., Biosenbsores and Bioelectronics, 154:112052 (2020) which relies on random chemical immbomilization of spyCatcher on the surfaces described therein. The coupling of COOH from the spyCatcher to the activated Cys on the surfaces in Oloketuyi, et al., Biosenbsores and Bioelectronics, 154:112052 (2020) is random because it can occur through any of the acidic residues (Asp or Glu) of the protein, or through more than one of them. There is therefore no control over the orientation of the spyCatcher. This then has three consequences: (1) loss of orientation control at that point; (2) a large fraction of the spyCatcher will not be able to react with the spyTag because of sterical or chemical blockage or unfolding, resulting in lower density coupling; and (3) loss of flexibility, since the spyCatcher is directly sticking to the surface (which is in contrast to the disclosed method wherein spyCatcher is swinging on a flexible linker above the OECT surface). The disclosed method of which attaches spyTag onto the gold surface first, followed by contacting the spy Tag-modified surface with a solution containing spyCatcher, overcomes these limitations. In contrast to the “spyCatcher first” approach, the disclosed method retains full orientation control throughout the immobilization stack. For example, it retains full spyCatcher integrity owing to the lack of chemical modification or adsorption and allows spyCatcher to swing on a flexible linker above the OECT surface. This maximizes the ability of the whole system to re-orient itself as needed for tight packing and binding of analyte.

A. Modification with a First Peptide

Generally, a first peptide (of a binding pair) is dissolved in a buffer as described above, such as PBS to form a first incubation composition. The first peptide is a modified peptide, which includes a short linker. Preferred linkers include CGGSGSGSG (SEQ ID NO: 22) and GSGC (SEQ ID NO:23). Exemplary modified first peptides (of a binding pair) include: CGGSGSGSGAHIVMVDAYKPTK (SEQ ID NO:24) and AHIVMVDAYKPTKGSGC (SEQ ID NO:25). The electrode surface is immersed in the first incubation composition containing the first peptide for a time period sufficient to couple the first peptide to OECT surface, such that a first peptide-modified OECT surface is produced. Typically, the incubation time period sufficient to couple the first peptide to the SAM on the surface of the electrode is in a range from 10 minutes to 24 hours, from 10 minutes to 20 hours, from 10 minutes to 15 hours, from 10 minutes to 12 hours, from 10 minutes to 10 hours, from 10 minutes to 5 hours, from 10 minutes to 2 hours, from 10 minutes to 1.5 hours, or from 10 minutes to 1 hour, such as about 1 hour.

The first peptide can couple to and conjugate with a second peptide as described below. An exemplary first peptide is SpyTag peptide, unmodified, or preferably, which is modified with a short linker (SEQ ID NO: 1) (disclosed in Zakeri, et al., Proc. Natl. Acad. Sci., 109:E690-E697 (2012)), preferably not modified with a maleimide functional group).

Typically, the concentration of the first peptide in the second incubation solution is in a range from 0.01 mg/mL to 20 mg/mL, from 0.01 mg/mL to 15 mg/mL, from 0.01 mg/mL to 10 mg/mL, from 0.05 mg/mL to 20 mg/mL, from 0.05 mg/mL to 15 mg/mL, from 0.05 mg/mL to 10 mg/mL, from 0.01 mg/mL to 5 mg/mL, from 0.01 mg/mL to 1 mg/mL, from 0.05 mg/mL to 5 mg/mL, or from 0.05 mg/mL to 1 mg/mL, such as about 0.1 mg/mL.

Optionally, following incubation, the first peptide-SAM-modified electrode surface is rinsed with a second rinsing composition, such as a buffer as described above.

### B. Method for Producing Biorecognition Element-Containing Fusion Proteins

Fusion proteins of Formula II can be obtained by, for example, by chemical synthesis, and more preferably, by recombinant production in a host cell. To recombinantly produce a fusion proteins of Formula II, a nucleic acid containing a nucleotide sequence encoding the polypeptide can be used to transform, transduce, or transfect a bacterial or eukaryotic host cell (e.g., an insect, yeast, or mammalian cell). In general, nucleic acid constructs include a regulatory sequence operably linked to a nucleotide sequence encoding a fusion proteins of Formula II. Regulatory sequences (also referred to herein as expression control sequences) typically do not encode a gene product, but instead affect the expression of the nucleic acid sequences to which they are operably linked. The nucleotide sequences encoding the fusion protein are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated. The vector is preferably an expression vector in which the DNA sequence encoding the fusion protein is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the fusion protein Expression vectors for use in expressing the fusion protein will comprise a promoter capable of directing the transcription of a cloned gene or cDNA. The promoter may be any DNA sequence, which shows transcriptional activity in the host cell of choice and may be derived from genes encoding proteins either homologous or heterologous to the host cell. Expression vectors for use in expressing the fusion protein will comprise a promoter capable of directing the transcription of a cloned gene or cDNA. The promoter may be any DNA sequence, which shows transcriptional activity in the host cell of choice and may be derived from genes encoding proteins either homologous or heterologous to the host cell. Examples of suitable promoters for directing the transcription of the DNA in mammalian cells are the SV40 promoter (Subramani et al., Mol. Cell. Biol 1 (1981), 854-864), the MT-1 (metallothionein gene) promoter (Palmiter et al., Science 222 (1983), 809-814), the CMV promoter (Boshart et al., Cell 41:521-530, 1985) or the adenovirus 2 major late promoter (Kaufman and Sharp. Mol. Cell. Biol, 2:1304-1319, 1982).

Useful prokaryotic and eukaryotic systems for expressing and producing polypeptides are well known in the art include, for example, Escherichia coli strains such as BL-21, and cultured mammalian cells such as CHO cells.

In eukaryotic host cells, a number of viral-based expression systems can be utilized to express fusion proteins of Formula II. Viral based expression systems are well known in the art and include, but are not limited to, baculoviral, SV40, retroviral, or vaccinia based viral vectors.

The expressed tagged or fusion proteins produced by the cells may be recovered from the culture medium by conventional procedures including separating the host cells from the medium by centrifugation or filtration, releasing the fusion protein by mechanical cell disruption, such as ultrasonication or pressure, precipitating the protein aqueous components of the supernatant or filtrate by means of a salt, e.g. ammonium sulphate. After sonication a suitable concentration of NaCl can be added to further decrease the ability of host cell contaminants to bind to the cation exchange matrix. After cation-exchange chromatography the fusion protein may be eluted in a salt gradient and eluate fractions containing the fusion protein are collected. In some preferred forms, fusion protein is captured from lysate through its His tag. So IMAC (immobilized metal affinity chromatography) was used and then, after concentration of protein-containing fractions, they are subjected to size exclusion chromatography (SEC) for final purification. In particularly preferred embodiments for nanobody purification, the nanobody is purified from the periplasmic space, where the host cell is bacteria, for example, E. coli. This would include (1) centrifugation, (2) osmotic shock to release the protein from the cell wall compartment, (3) IMAC (Immobilized Metal Ion Affinity Chromatography), (4) SEC (Size Exclusion Chromatography).

C. Modification with Biorecognition Elements

Generally, a second peptide-biorecognition element conjugate and a blocking agent are dissolved in a blocking buffer solution, including a buffering agent such as HEPES, to form a second incubation solution. Preferably, the blocking agent contained in the second incubation solution is BSA.

Typically, the concentration of the blocking agent in the second incubation solution is in a range from 0.01% w/v to 10% w/v, from 0.01% w/v to 5% w/v, from 0.01% w/v to 1% w/v, from 0.05% w/v to 10% w/v, from 0.05% w/v to 5% w/v, or from 0.05% w/v to 1% w/v, such as about 0.1% w/v. The first peptide-modified electrode surface is immersed in the second incubation solution containing the second peptide-biorecognition element conjugate and the blocking agent. Generally, the second peptide of the peptide-biorecognition element conjugate is capable of conjugating with the first peptide to form a biomolecular linker and thus attach the biorecognition element on the electrode surface. An exemplary second peptide is SpyCatcher peptide as disclosed in Zakeri, et al., Proc. Natl. Acad. Sci., 109: E690-E697 (2012). For example, a SpyTag/SpyCatcher linker is formed to control the oriented immobilization of the biorecognition elements (e.g., nanobodies) on the electrode surface. After first peptide binding partner-second peptide binding partner linker formation, the biorecognition element (e.g., nanobody) is functionalized on the gate electrode surface of the OECT, in a configuration vertical to electrode surface and is vertical relative to the channel surface, allowing for binding with an analyte in a sample, when the sample contacts the biorecognition element-functionalized electrode. The biorecognition elements can vary depending on the analytes of interest. For example, the biorecognition element is a nanobody such as an anti-GFP nanobody or an anti-SARS-1-RBD nanobody or an anti-MERS RBD nanobody (See TABLE 1).

TABLE 1
Protein sequences

| Construct | | |
|---|---|---|
| Name | Description | Sequence |
| Peptide | MCA-SpyTag | SGSGAHIVMVDAYKPTK (SEQ ID NO: 1) (linker sequence |
| | | is underlined) |
| GFP | GFPnanobody-8 aa- | MASQVQLVESGGALVQPGGSLRLSCAASGFPVNRYSMRW |
| Nanobody | Spy Catcher-3C | YRQAPGKEREWVAGMSSAGDRSSYEDSVKGRFTISRDDAR |
| | cleavage site-His₁₀ | NTVYLQMNSLKPEDTAVYYCNVNVGFEYWGQGTQVTVSS |
| | | KGSGSGSGSVDTLSGLSSEQGQSGDMTIEEDSATHIKFSKRD |
| | | EDGKELAGATMELRDSSGKTISTWISDGQVKDFYLYPGKY |
| | | TFVETAAPDGYEVATAITFTVNEQGQVTVNGKATKGDAHI |
| | | SGLEVLFQGPTGHHHHHHHHHH (SEQ ID NO: 2) |
| SARS- | VHH72-8 aa- | MTGQVQLQESGGGLVQAGGSLRLSCAASGRTFSEYAMGW |
| CoV-1/2 | spy Catcher-3C | FRQAPGKEREFVATISWSGGSTYYTDSVKGRFTISRDNAKN |
| nanobody | cleavage site-His₈ | TVYLQMNSLKPDDTAVYYCAAAGLGTVVSEWDYDYDYW |
| (VHH72) | | GQGTQVTVSSGSGSGSGSGSVDTLSGLSSEQGQSGDMTIEE |
| | | DSATHIKFSKRDEDGKELAGATMELRDSSGKTISTWISDGQ |
| | | VKDFYLYPGKYTFVETAAPDGYEVATAITFTVNEQGQVTV |
| | | NGKATKGDAHISGLEVLFQGPTGHHHHHHHH (SEQ ID |
| | | NO: 15) |
| MERS- | VHH04-8 aa- | MTGEVQLQESGGGSVQAGGSLRLSCEASGTISSMYCMGWF |
| CoV | spy Catcher-3C | RQAPGKEREGVALFNRSTGVEYYRASVKGRFTISHDNAKN |
| nanobody | cleavage site-His₁₀ | TVYLQMNSLKLEDTAVYYCAAGPTCGGWYPGLYNYWGQ |
| (VHH04) | | GTQVTVSSGSGSGSGSVDTLSGLSSEQGQSGDMTIEEDSAT |
| | | HIKFSKRDEDGKELAGATMELRDSSGKTISTWISDGQVKDF |
| | | YLYPGKYTFVETAAPDGYEVATAITFTVNEQGQVTVNGKA |
| | | TKGDAHISGLEVLFQGPTGHHHHHHHHHH (SEQ ID NO: |
| | | 17) |
| Target | snoop Tag-msfGFP- | MASKLGDIEFIKVNKGSGSGSGSVSKGEELFTGVVPILVELD |
| (msfGFP) | 3C cleavage site- | GDVNGHKFSVRGEGEGDATNGKLTLKFICTTGKLPVPWPT |
| | TwinStrep | LVTTLTYGVQCFSRYPDHMKQHDFFKSAMPEGYVQERTIS |
| | | FKDDGTYKTRAEVKFEGDTLVNRIELKGIDFKEDGNILGHK |
| | | LEYNFNSHNVYITADKQKNGIKANFKIRHNVEDGSVQLAD |
| | | HYQQNTPIGDGPVLLPDNHYLSTQSKLSKDPNEKRDHMVL |
| | | LEFVTAAGITHGMDELYKTGSTGLEVLFQGPTGSAWSHPQ |
| | | FEKGTGSGTGSGTGSWSHPQFEK (SEQ ID NO: 3) |

The conjugation between SpyTag and SpyCatcher is a robust method for conjugating the recombinant proteins where the peptide SpyTag can spontaneously react with the protein SpyCatcher in a facile manner and with high specificity (Zakeri, et al., Proc. Natl. Acad. Sci., 109: E690-E697 (2012); Keeble et al., 2017). An additional advantage of the disclosed techniques is that there is no need for maleimide functional groups to be constructed in the SpyTag peptide. SpyTag peptide can be chemically synthesized by any of the known methods described above. The nanobodies as fusion to the SpyCatcher proteins, can be expressed as fusion protein in any suitable protein expression system, preferably with a tag such as a His tag, to aid in its purification.

The first peptide-modified electrode surface is incubated with the second incubation solution for a time period sufficient to form the linker (i.e. first peptide/second peptide conjugate), such that a biorecognition element-linker-SAM modified electrode surface is produced (biorecognition layer integrated on the electrode surface.

Typically, the incubation time period sufficient to form the second peptide/first peptide conjugate on the surface of the electrode is in a range from 5 minutes to 24 hours, from 10 minutes to 20 hours, from 10 minutes to 15 hours, from 10 minutes to 12 hours, from 10 minutes to 10 hours, from 10 minutes to 5 hours, from 10 minutes to 2 hours, from 10 minutes to 1.5 hours, or from 10 minutes to 1 hour, such as about 1 hour.

Typically, the concentration of the second peptide-recognition element conjugate in the second incubation solution is in a range from 1 μM to 100 μM, from 1 μM to 100 μM, from 1 μM to 90 μM, from 1 μM to 80 μM, from 10 μM to 100 μM, from 20 μM to 90 μM, from 10 μM to 80 μM, from 5 μM to 100 μM, from 5 μM to 90 μM, from 5 μM to 80 μM, from 20 μM to 100 μM, from 20 μM to 90 μM, or from 20 μM to 80 μM, such as about 50 μM.

Optionally, following incubation, the biorecognition layer-integrated electrode surface is rinsed with a third rinsing solvent, such as a buffer as described above.

### D. Exemplary Methods of Electrode Functionalization

An exemplary method of preparing a biorecognition layer-integrated electrode surface is described in the Examples.

A preferred method uses p-type depletion (PEDOT:PSS) and accumulation (such as p(g0T2-g6T2)) mode materials in the OECT channel while the gate electrode is gold. The gate electrode (preferably gold) surface is functionalized with a biorecognition layer, i.e., the biorecognition element, for example, a nanobody. The gate electrode surface is electrochemically cleaned and then exposed to the first and second incubation compositions as disclosed above.

The disclosed methods above result in OECT devices containing a biorecognition element whose binding partner is any analyte of interest, for example an antigen from any pathogen.

### III. Methods of Using

OECT devices containing Cas protein with a gRNA whose binding partner is any RNA of interest, for example RNA from any pathogen can be used to detection pathogens of interest in a sample. OECT devices containing Cas protein with a gRNA whose binding partner is any RNA of interest, for example RNA from any pathogen can be used to detection pathogens of interest in a sample.

### A. Directly Functionalized OECT

The disclosed device is used to measure the presence, the absence, or the concentration of any analyte in a sample, where the analyte is a binding partner to the biorecognition element functionalized onto the device. The biorecognition element functionalized-OECT Sensor features: improved sensitivity high sensitivity over prior art devices (aM or zM compared to fM) and selectivity, delivering accurate results in accordance with gold standard tests (where possible and applicable, correlated to DNA or RNA detection results with the conventional PCR method); fast detection time and high limit of detection (lower limit of detection (LOD) exemplified below forSARS-CoV-2 S1 in saliva is 6×10⁻²² M). Furthermore, the peptide-based biorecognition element (which eliminates Chem-SAM) gates show improved chemical and electrochemical stability compared to the HDT SAM-based gates

The OECT sensor relies on the channel made from or containing ion-permeable organic electronic material, through which holes or electrons flow from the source electrode to the drain electrode. When ions are injected from the electrolyte solution into the channel, its doping state is changed and thus its conductivity is changed. The operation of OECT is controlled by voltages applied to the gate electrode (gate voltage, VG) and to the drain electrode (drain voltage, VD), which are referenced with respect to the source electrode. The drain voltage induces a current (drain current, ID), which is proportional to the quantity of mobile holes or electrons in the channel, and thus probes the doping state of the organic electronic material. The gate voltage controls the injection of ions into the channel and thus the doping state of the organic electronic material, resulting in a change in ID. The change in ID may be expressed as a normalized response (also referred herein as “NR”) which can be calculated based on equation 1:

NR=(|ID−I0|)/I0
where I0 is the drain current obtained after incubation of the OECT with a blank solution (i.e. a solution in the absence of the target analytes) under the same measurement conditions as the biological sample.

As the biorecognition element for example, nanobody selectively captures its target, exemplified herein using GFP protein and SAR-CoV-2 protein, this binding event changes the capacitance of the gate electrode and induces the potential across the gate/electrolyte interface, suppressing the gating of the OECT. Therefore, the OECT signal varies depending on the concentration of analyte in the sample down to femtomolar and attomolar range. This strategy of utilizing nanobody units opens up a new avenue for the design of electronic biosensors and can be further expanded into different sensing platforms and various target proteins.

Generally, a method of using the disclosed device for testing the presence, the absence, and/or concentration of analytes in a biological sample includes: (i) incubating the gate electrode (functionalized with a biorecognition elements as disclosed above) with a blank solution, for example a buffer like PBS, universal transport medium (UTM), or virus transport medium (VTM), (ii) placing the gate electrode on top of the channel, wherein the channel is in contact with an electrolyte solution, (iii) applying a VG and a VD; (iv) measuring a first ID (also referred herein as a background ID); (v) incubating the gate electrode with the biological sample for a time period sufficient to allow binding between the analyte and the biorecognition element; (vi) rinsing the gate electrode with a rinsing buffer; and (vii) measuring a second ID (also referred herein as a signal ID), where a difference between the second ID and the first ID is indicative of the absence, the presence, or the concentration of the analyte in the biological sample. The second ID may be larger, the same or substantially the same as, or smaller than the first ID. Optionally, steps (v)-(vii) are repeated one or more times. In some embodiments, the steps (i)-(iv) are optional and the background ID is provided to the user otherwise, such as by including calibration or standard data in an operation manual.

Optionally, the method includes a step of adding an electrolyte solution into the reservoir prior to any one of steps (i)-(vii) described above, such as prior to step (v) or prior to step (vi) and subsequent to step (v). The electrolyte solution added into the reservoir can be any electrolyte solution described above, for example, a PBS at pH about 7.4. The blank solution may be a buffer solution that does not contain the analyte of interest and is used to establish a baseline drain current, i.e. I0. Typically, a drop of the blank solution is applied onto the gate electrode and incubated for about 10 minutes. Generally, the time period for incubating the gate electrode with the blank solution is the same as the incubation time period sufficient to allow binding between the analyte and the biorecognition element. Optionally, the gate electrode is rinsed with a rinsing buffer following incubation with the blank solution for about 10-15 second. The rinsing buffer may be any electrolyte solution as described above, such as PBS.

Typically, the VG and VD are applied simultaneously to the gate electrode and the drain electrode, respectively. In some embodiments, the VG is applied to the gate electrode by sweeping from a first gate voltage to a second gate voltage at a gate voltage step and the VD is applied to the drain electrode by sweeping from a first drain voltage to a second drain voltage at a drain voltage step. For example, the VG is applied to the gate electrode by sweeping from −1 V to 1 V, from −0.8 V to 0.8 V, or from −0.6 V to 0.6 V, at a gate voltage step of 0.05 V, 0.1 V, 0.2 V, or 0.5 V and the VD is applied to the drain electrode by sweeping from 0 V to 1 V, from 0 to 0.6 V, from 0.5 V to −1 V, from 0 V to −1 V, from 0 V to −0.8 V, or from 0 V to −0.6 V, at a drain voltage step of 0.05 V, 0.1 V, 0.2 V, or 0.5 V. In some embodiments, the VG is applied to the gate electrode by sweeping from −0.6 V to 0.6 V, from 0.2 V to −0.4 V, or from 0.2 V to −0.6 V at a gate voltage step of 0.1 V or 0.05 V and the VD is applied to the drain electrode by sweeping from 0 V to −0.6 V, from 0 V to −0.4 V at a drain voltage step of 0.1 V or 0.05 V. In some embodiments, the VG is applied to the gate electrode by sweeping from 0 V to 0.9 V or from 0 V to 0.6 V at a gate voltage step of 0.1 V or 0.05 V and the VD is applied to the drain electrode by sweeping from 0 V to 0.6 V at a drain voltage step of 0.1 V or 0.05 V. When both VG and VD are applied by sweeping, a range of IDs can be collected.

In some embodiments, a fixed VG is applied to the gate electrode and a fixed VD is applied to the drain electrode. The fixed VG and fixed VD may be the same or different. For example, a fixed VG in the range from −1 V to 1V, from −0.6 V to 0.6 V, or from −0.1 V to 0.1 V, such as −0.6V, −0.1 V, or 0.6 V is applied to the gate electrode, and a fixed VD in the range from −1 V to 1V, from −0.6 V to 0.6 V, 0 V to −0.6 V, or from −0.1 V to 0.1 V, such as −0.6 V, −0.1 V, or 0.6 V, is applied to the drain electrode. In some embodiments, a VG of −0.1 V is applied to the gate electrode and a VD of −0.1 V is applied to the drain electrode. In some embodiments, a VG of 0.1 V is applied to the gate electrode and a VD of 0.1 V is applied to the drain electrode. In some embodiments, a VG of −0.1 V is applied to the gate electrode and a VD of 0.1 V is applied to the drain electrode. In some embodiments, a VG of 0.1 V is applied to the gate electrode and a VD of −0.1 V is applied to the drain electrode. In some embodiments, a VG of −0.6 V is applied to the gate electrode and a VD of −0.6 V is applied to the drain electrode. In some embodiments, a VG of 0.6 V is applied to the gate electrode and a VD of 0.6 V is applied to the drain electrode. In some embodiments, a VG of 0.6 V is applied to the gate electrode and a VD of −0.6 V is applied to the drain electrode. In some embodiments, a VG of −0.6 V is applied to the gate electrode and a VD of 0.6 V is applied to the drain electrode. In some embodiments, a VG of 0.5 V is applied to the gate electrode and a VD of 0.1 V is applied to the drain electrode. In some embodiments, a VG of 0.9 V is applied to the gate electrode and a VD of 0.1 V is applied to the drain electrode. When a fixed VG and a fixed VD are applied, a single ID can be collected.

The biological sample is typically in a liquid form and applied onto the gate electrode as a drop and incubates for a period of time sufficient to allow binding between the analytes and the biorecognition element (e.g. nanobody) immobilized on the gate electrode of the OECT. Typically, the volume of the biological sample sufficient for incubation is small. For example, the volume of the biological sample sufficient for incubation is less than 20 μL, less than 10 μL, or preferably, ≤5 μL, and more preferably, about 5 μL.

The biological samples can be a bodily fluid, such as whole blood, plasma, serum, saliva, nasal swab, mucus, sputum (processed or unprocessed), bronchial alveolar lavage (BAL), bronchial wash (BW), cerebrospinal fluid (CSF), and urine.

In some embodiments, the biological sample is not a bodily fluid, but is a liquid obtained from a solid specimen, such as tissue (e.g., biopsy material), feces, rectal swab, nasopharyngeal swab, and throat swab. When the biological sample is not a bodily fluid, the above-described exemplary method can be modified to include an initial step of processing the specimen t to obtain a sample in liquid form, which is then subjected to steps (i)-(v) described above for a method of using the disclosed device for testing the presence, the absence, and/or concentration of analytes in a biological sample. Processing methods to transform a specimen (which is not a fluid) into a liquid form are known. For example, when the specimen is a nasopharyngeal swab, it is processed by placing the proximal portion of the swab in a buffer to produce the biological sample in a liquid form (Lopez, et al., Pediatr. Res., 86 (5): 651-654 (2019)). Optionally, when specimen processing is needed, the method also includes a desalting step prior to specimen processing, where the buffer is run through a desalting column to remove any redox reagents, such as dithiothreitol (DTT), to reduce interference signals caused by non-specific interactions of the redox reagents with the gate electrode surface. Optionally, the desalting step is performed following specimen processing and prior to step (v), where the obtained biological sample in a liquid form is run through a desalting column to remove any redox reagents introduced during the specimen processing step.

In some embodiments, the above-described exemplary method includes a step of adding a protease inhibitor into the biological sample prior to other steps, such as prior to any one of steps (i)-(v), particularly prior to step (v). The protease inhibitor can prevent or reduce the damage to the sensor's protein-based recognition layer caused by protease activity in the biological or environmental sample.

The biological sample is incubated with the gate electrode for a time period sufficient to allow binding between the analytes in the biological sample and the biorecognition element (e.g. nanobody) modified on the gate electrode surface. For example, the incubation time period is up to 60 minutes, up to 50 minutes, up to 40 minutes, up to 30 minutes, up to 20 minutes, or up to 10 minutes, for example, 5, 6, 7, 8, 9 or 10 minutes. For example, the biological sample is incubated with the gate electrode for about 10 minutes to allow binding between the analytes in the biological sample and the nanobody modified on the gate electrode surface. The incubation may be performed statically, under shaking, or with up and down pipetting, and in some embodiments, for about 3-5 minutes. In some preferred embodiments, the incubation is performed with pipetting to facilitate binding between the analytes in the biological sample and the biorecognition element (e.g. nanobody) modified on the gate electrode surface and thereby reduce the time needed for the incubation. When pipetting is performed during incubation, the biological sample is mixed up and down with a pipette for a time period in a range from 5 seconds to 1 minute, from 5 seconds to 50 seconds, from 5 seconds to 40 seconds, from 10 seconds to 1 minute, from 10 seconds to 50 seconds, from 10 seconds to 40 seconds, or from 20 seconds to 1 minute, such as about 30 seconds and the pipetting is repeated for at least one time or at least two times. For example, during a 10-minute incubation, the biological sample is mixed up and down with pipetting for about 30 seconds and the pipetting is performed for a total of 3 times (i.e. 3×30-second pipetting or a 30-second pipetting every 3 minutes during the 10-minute incubation period).

Following this incubation step (v), the gate electrode is rinsed with a rinsing buffer to remove any unbounded analytes, for example, it can be rinsed for up to 1 min, 30 secs, 15 secs etc., and in some embodiments, it is rinsed by dipping in (for about 15 secs) and out of the rinsing buffer, and repeating this, twice. The rinsed gate electrode is then brought back in contact with the electrolyte solution prior to step (vii) measuring a second ID. The rinsing buffer may be any electrolyte solution as described above, such as PBS.

Optionally, the above-described method includes a step of exposing the gate electrode surface to a glycine solution at a pH less than 5, less than 4, or less than 3, such as about 2, following step (vii). The acidic glycine solution can disrupt the binding between the analyte and the recognition element (e.g. the analyte/nanobody binding) and thus regenerate the OECT sensor for further sample measurements. For example, following the regeneration step, the ID generated from the OECT is comparable to the I0 (i.e. the difference between the ID obtained after regeneration and I0 is less than about 10%).

The exemplified methods can be used to make OECT devices containing a biorecognition element such as a nanobody whose binding partner is any analyte of interest, for example an antigen from SAR-CoV-2, such as the spike protein. Specific nanobodies against SARS-CoV-2 Spike protein (S protein) or, more specifically, against the Receptor Binding Domain (RBD) within the S protein are available. Two nanobodies that were originally targeting SARS-1 RBD have very recently been shown to also recognize SARS-CoV-2 RBD with high affinity9. There are other nanobodies, engineered from a human antibody framework, that were specifically developed for SARS-CoV-2 (Wu, et al., Fully human single-domain antibodies against SARS-CoV-2. bioRxiv 2020). The human ACE2 receptor protein, to which the virus binds with high affinity, can be used an alternative recognition module. The subject can be symptomatic or asymptomatic. In some embodiments, the subject has been exposed to the virus, however, the subject may have no known exposure to the virus.

### B. Convection Driven Ultra-Rapid Detection of Biomarkers

In some embodiments, the disclosed electrochemical transistor (OECT) is incorporated with the micro-stirring effect of alternating current electrothermal flow (ACET) for the ultra-rapid detection of single-molecule-to-nanomolar levels of the severe acute respiratory syndrome coronavirus 2 (SARS-CoV-2) in complex bodily fluids.

An effective method for decreasing the incubation time while improving the sensitivity can be achieved by generating directional convective microflows that transport the analyte to functionalized sites. Alternating current (AC) electrokinetic/electrohydrodynamic forces are employed as an effortless method for an electrode/electrolyte system to accelerate the transport of biological species to the sensor surface for enhanced immunocomplex formation. Especially for a biologically relevant high-conductivity media, AC electrothermal (ACET) flow becomes the dominant phenomenon among the other AC electrokinetic/electrohydrodynamic forces and induces directional, long-range convective vortices that can deliver the target proteins to the electrode surface. Then, it conveys nonspecific ones tangentially away to the electrode surface. Methods of analyte detection via AC electrokinetic/electrohydrodynamic forces combined with an OECT-based immunosensor are disclosed herein.

The method can follow the steps and conditions as described above for using a directly functionalized OECT sensor, which include: (i) optionally incubating the gate electrode (functionalized with a biorecognition elements as disclosed above) with a blank solution, for example a buffer like PBS, universal transport medium (UTM), or virus transport medium (VTM), (ii) optionally placing the gate electrode on top of the channel, wherein the channel is in contact with an electrolyte solution, (iii) optionally applying a VG and a VD; (iv) optionally measuring a first ID; (v) incubating the gate electrode with the biological sample for a time period sufficient to allow binding between the analyte and the biorecognition element; (vi) rinsing the gate electrode with a rinsing buffer; and (vii) measuring a second ID, where a difference between the second ID and the first ID is indicative of the absence, the presence, or the concentration of the analyte in the biological sample. The second ID may be larger, the same or substantially the same as, or smaller than the first ID. Optionally, steps (v)-(vii) are repeated one or more times. Optionally, the method includes a step of adding an electrolyte solution into the reservoir prior to step (i) or prior to step (ii) and subsequent to step (i).

In addition to these steps, the method using AC electrokinetic/electrohydrodynamic forces for analyte detection further includes a step of applying an AC potential to the conductive layer, optionally performed prior to and/or during step (v) and optionally prior to and/or during step (i). The AC potential applied to the conductive layer (1) is sufficient to generate the micro-mixing effect (e.g., reaching an average water velocity of at least 15 μm s⁻¹), and thereby rapidly transport target molecules to the sensor surface and allow the target molecules (such as Spike protein) to accumulate on the immobilized binding partners (such as nanobodies) at the gate electrode and (2) maintains the temperature of the electrolyte solution (change of solution temperature is only a few degrees Celsius, such as less than about 8 degrees Celsius, less than 6 degrees Celsius, or less than 4 degrees Celsius). Generally, the AC potential being applied to the conductive layer can be in a range from 1 Vpp to 8 Vpp or from 2 Vpp to 6 Vpp, such as about 6 Vpp (pp refers to peak to peak).

Alternating current electrothermal flow (ACET) is used as a tool to reduce the detection time of transistor-based biosensors. The ACET flow causes a micro stirring effect for rapid detection of single-molecule-to-nanomolar levels of biomarkers in complex bodily fluids. ACET flow is induced by an AC electric field applied on the gate electrode and it moves the analyte onto the concentric gate electrode within a minute. The analyte is then captured by the recognition units on the gate electrode while the non-target species are moved away from the detection area.

The OECT, designed and run as disclosed herein provides results after less than 5 mins, such as after 2-min or after 1-min, of exposure of the sample (without manual pipetting), which is otherwise taking about 1 hour, from sample incubation-to-result with passive diffusion, and at least 10-15 mins with manual pipetting. The increase in detection speed does not decrease the detection quality; the ACET integrated OECT maintains high specificity and single-molecule sensitivity in the buffer and biological sample, such as saliva. The ACET-enhanced, OECT-based sensor can also achieve a higher specificity and lower background due to electrothermal flow-induced removal of nonspecific species from the sensor surface, compared to the same OECT-based immunosensor without ACET. Additionally, the ACET-enhanced, OECT-based immunosensors show a low energy consumption (i.e., nW level, such as about 100 nW). For example, the ACET-enhanced, OECT-based sensors can be operated using only 100 nW power and about 2 min of incubation with a 10 μL or 5 μL sample for detecting target analytes in complex media with single-molecule sensitivity.

A particularly preferred embodiment is exemplified below.

### EXAMPLES

### Example 1. Directly Functionalized OECT

### Materials and Methods

### Materials

Sodium chloride, Tween-20, glycerol, HEPES, bovine serum albumin (BSA), 1,6-hexanedithiol (HDT), and PBS (pH 7.4) were purchased from Sigma-Aldrich and used as received. All aqueous solutions were prepared with ultrapure water (Millipore Milli-Q). p(g0T2-g6T2) was synthesized as reported previously. Protein purification materials: Agar, LB broth, 2×YT broth, kanamycin, glucose, isopropyl β-D-1-thiogalactopyranoside (IPTG), BugBuster (Novagen), complete protease inhibitor cocktail (Sigma), benzonase (Novagen), hen egg white lysozyme (Fluka), tris(2-carboxyethyl)phosphine (TCEP), tris(hydroxymethyl)aminomethane hydrochloride (Tris-HCl), imidazole, glycerol, dithiothreitol (DTT), ethylenediaminetetraacetic acid (EDTA), D-desthiobiotin, 10K Amicon ultra spin concentrators (Milipore). Purification columns and materials were purchased from GE. Healthcare: His Trap-HP 5 ml, StrepTrap-HP 5 ml, Superdex75 16/600. Cysteine terminated spyTag peptide and the MCA-spyTag peptide were synthesized by GenScript Biotech (Singapore). SARS-CoV-2 S1 (40591-V08B1) was purchased from Sino Biological. Universal transport medium kit (UTM, proprietary composition) was obtained from Noble Biosciences, Inc. The SARS-CoV-2 Spike-pseudotyped lentivirus (CAT. SP101-100) and the non-typed lentivirus negative control (CAT. SP401-025) were purchased from GeneCopoeia Inc. Untreated wastewater was collected from KAUST wastewater treatment plant. Saliva was collected from volunteers. The protocols and procedures involving human saliva, were approved by the KAUST IBEC (under approval numbers 18IBEC11 and 20IBEC25) and National Committee of BioEthics, Saudi Arabia (registration number HAP-02-J-042).

### OECT and Gate Electrode Fabrication

OECTs were fabricated photolithographically using a parylene-C(PaC) peel-off method, as reported previously. p(g0T2-g6T2) films were spun coated (800 rpm, 45 s) from a chloroform solution (5 g/L) on the substrates to yield a film thickness of about 70 nm in the channel. Then the second PaC layer was removed by tape.

The gate electrodes were fabricated using flexible substrate polyimide (175 μm of thickness). A Cr layer with thickness of 10 nm as an adhesion promoter and gold layer with a thickness of 180 nm were sputtered on the substrate. Then the gold-coated substrate was cut with Silhouette Cameo into a square geometry with a defined area (0.64 or 4 mm²). All the electrodes were cleaned by sonication in isopropyl alcohol, deionized water, and dried in vacuum oven overnight. The gate electrode was electrochemically cleaned in 10 mM sulfuric acid using cyclic voltammetry (CV). 20 CVs were applied with a potential range between −0.2 V and 1.2 V at a scan rate of 100 mV s⁻¹.

### Biofunctionalization of SpyDirect-Gate Electrode

First, 0.1 mg/mL cysteine terminated peptide with spyTag linker were dissolved in water and applied to the gate electrode for 1 h. The electrodes were rinsed thoroughly with water. Second, 50 μM green fluorescent protein (GFP) or SARS-CoV-1 nanobody (with spyCatcher) were dissolved in binding buffer (20 mM HEPES pH 7.4, 150 mM NaCl, 0.02% w/v NaN₃, 0.05% v/v Tween-20, 0.1% w/v BSA), and incubated with the peptide-linked electrodes for 1 h. Subsequently, the nanobody functionalized gate electrodes were rinsed with binding buffer.

### Electrochemical Measurements

All electrochemical measurements were performed in a conventional three-electrode setup using a potentiostat (Autolab PGstat128N, MetroOhm). A platinum wire and Ag/AgCl electrodes were employed as the counter electrode and reference electrodes, respectively. A gold electrode was used as working electrode. The electrochemical characteristics of the gold electrode were investigated before and after the surface modifications with different reagents by electrochemical impedance spectroscopy (EIS) and CV. Measurements were carried out in 5 mL of 10 mM PBS solution (pH 7.4) containing 10 mM [Fe(CN)₆]^{3-/4-}. For CV measurements, the potential window of gold was determined typically between-0.2 and 0.6 V and the scan rate was kept at 10 mV s⁻¹. Impedance spectra were recorded at a DC voltage of 0 V versus open circuit potential and an AC modulation of 10 mV over a frequency range of 0.1-100000 Hz.

### X-Ray Photoelectron Spectroscopy (XPS) Characterization.

XPS measurements were performed using AMICUS/ESCA (1468.6 eV). The source was operated at 10 kV with 10 mA current generating a power of 100 W. The vacuum level of the analysis chamber was maintained at 10⁻⁷ Pa during the measurements. The obtained spectra were calibrated to reference of C1s at 284.8 eV. The XPS spectra were deconvoluted using Gaussian and Lorentzian methods and background subtraction was carried out by Tougaard method.

Quartz Crystal Microbalance with Dissipation (QCMD) Monitoring

QCMD measurements were conducted using a Q-sense analyzer (QE401, Biolin Scientific) by following either HDT SAM- or SpyDirect-biofunctionalization. The piezoelectrically active gold sensors (0.7854 cm²) were used. All the solution was injected into the chamber with a flow rate of 100 μL/min, controlled by a peristaltic pump. After ensuring that the sensor was fully covered with the solution, the pump was stopped for static incubation for certain period time. All QCMD data presented herein were recorded at the 7th overtone and analyzed using the same method detailed in previous work.

### Atomic Force Microscopy (AFM)

AFM scans were obtained with a Veeco Dimension 3100 Scanning Probe System. In electrolyte topographic scans were conducted using the Bruker SCANASYST-FLUID module mounted with Scanasyst-fluid probes commercialized by Bruker (nominal resonant frequency: 150 kHz, spring constant: 0.7 N m⁻¹). Sample and probe were both immersed in 10 mM PBS, pH 7.4 at room temperature while scanning. Gwyddion software was used for statistical data and post-treatment.

### Proteins

Nanobody-spyCatcher fusion proteins were designed based on previous study. SARS-CoV-2 spike protein was thawed on ice and centrifuged at 14.000 rpm at 4° C. for 45 min to remove potential aggregates (although no aggregation was observed). SARS-CoV-2 S1 was used as received from Sino Biologicals for the preparation of a dilution series. Equivalent dilutions of the Sino Biologicals storage buffer in itself were used as negative control. Lab-produced proteins were desalted into DTT-free storage buffer (20 mM HEPES pH 7.4, 150 mM NaCl, 0.05% v/v Tween-20, 0.02% w/v NaN₃) before use. Protein concentrations were assessed spectrophotometrically (Nanodrop, Thermofisher). Protein dilutions were prepared in standard sensor binding buffer (20 mM HEPES pH 7.4, 150 mM NaCl, 0.05% v/v Tween-20, 0.02% w/v NaN₃, 0.1% w/v BSA). For measurement of saliva samples, a complete protease inhibitor cocktail with EDTA (Sigma) was added at 4 times the concentration recommended by the manufacturer (resulting in a two-fold concentration in the final 1:1 mixture with saliva), and 0.5% w/v BSA was included (buffer 3.2). For measurements in the standard binding buffer, 4-fold dilution series were prepared in 96-well microplates over 23 steps starting from 320 nM. For measurements in saliva, target protein dilution series were prepared in the appropriate buffer (standard or saliva binding buffer) starting from 640 nM so that final concentrations were identical after 1:1 mixture with saliva.

### SARS-CoV-2 Pseudotyped Lentivirus

According to the supplier, the concentration of SARS-CoV-2 pseudotyped lentivirus is of 8.28×10¹⁰ copies/ml (determined by RT-qPCR) while the concentration of the negative control was 1.39×10¹¹ copies/ml (determined by RT-qPCR). Stock samples were thawed on ice and used as-is for the preparation of a dilution series starting at 1×10¹⁰ copies/ml in binding buffer (20 mM HEPES pH 7.4, 150 mM NaCl, 0.05% v/v Tween-20, 0.02% w/v NaN₃) enriched for saliva measurements to include 0.5% w/v BSA and 4 times the manufacturer-recommended concentration of complete protease inhibitor cocktail with EDTA (Roche). UTM and wastewater were diluted.

### OECT Sensor Characterization and Operation

Electrical characterization of the transistor was carried out with Keithley source meter, which was used to apply the drain and gate voltage while the source electrode functioned as the common ground in both circuits. All the measurements were conducted under ambient conditions. A PDMS well was glued on top of the channels and filled with 200 μL of 40 mM phosphate buffer (PB) as electrolyte. The OECT channel was stabilized with reference Ag/AgCl gate by repeating output curves (IVs). The steady-state measurements of the OECTs were performed by acquiring drain current (I_{D}) vs. drain voltage (V_{D}) at gate voltages (V_{G}) varying in between 0.2 and −0.4 V (step 0.05 V). VD was swept from 0 to −0.4 V. For each measurement, 5 IVs were acquired and the fifth IV was used to calculate the NR. All the nanobody gates were kept in PB (40 mM, pH 7.4) for at least 10 min to stabilize the sensor before sensing. A baseline in blank was obtained prior to sensing, the read-out signals obtained were used as baseline (gₘ₀) The nanobody functionalized gate electrode was incubated at room temperature for 10 min (pipetting 30 s every 3 min) with 5 μL sample solution. The normalized response (NR) was used to determine a calibration curve according to the following equation:

NR=|(g_{mD}−gₘ₀)|/gₘ₀ (4).

### Results

### Sensor Overview

The sensor contains an OECT channel and a SpyDirect nanobody functionalized gate electrode (FIGS. 1A-1C). A p-type conjugated polymer p(g0T2-g6T2) (structure of which is shown below) is spun-cast on the OECT channel, allowing the OECT to function in accumulation mode.

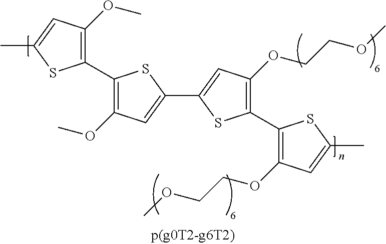

The OECT is initially in its OFF state in the absence of a gate voltage. Once a negative bias is applied, anions from the electrolyte are injected into the film and compensate the holes, thus leading to the ON state. The high operational stability of the OECT device was proved with standard Ag/AgCl reference gate electrode. The output characteristics of the OECTs were recorded using 10 mM PBS as electrolyte. At low VD, the increase in ID is significant, followed by a saturation regime at higher VD, consistent with accumulation mode OECT operation. The device showed minimal hysteresis with almost identical behavior, as observed from forward and backward voltage scans (data not shown). The p(g0T2-g6T2) transistors had low OFF-currents on the order of 10 μA, and an ON/OFF ratio of up to 100 at gate voltages which lead to maximum gₘ in the saturation regime (data not shown). The accumulation mode OECT had a lower power demand (75 μW at V_{G}=−0.05 V, V_{D}=−0.1 V) when operated at the subthreshold regime which yields the maximum NR values (data not shown). The operational stability of the devices was evaluated by switching them “ON” and “OFF” for 10 s each and recording the ID over 360 cycles performed within 2 hours (data not shown). The device retained 98% of its initial current, proving that it is highly stable in an aqueous media under the electrical stimulation at V_{G}=V_{D}=−0.5V. Moreover, small gate voltages applied to keep the device in its ON-state reduces the risk of material instability for long term use requirements and outstanding stabilities can be achieved at device IV [symbol] operation conditions. Atomic force microscopy (AFM) was used to confirm the change of the surface roughness and feature height during biofunctionalization (data not shown). Before immobilizing any biomolecules, the root mean square (RMS) roughness of the Au electrode is 4.5 nm and the mean height of the Au grains is 14.7 nm (data not shown). After incubating with the nanobody solution containing BSA, large particles were observed on the gate electrode. The RMS roughness of the nanobody/BSA modified gate electrodes increase to 6.0 nm. And the feature height of these added biomolecule layer increased 8.4 nm (from 14.7 to 23.1 nm), confirming the immobilization of nanobody and BSA.

The sensor response was normalized from the transfer characteristics. A measurement in blank was obtained prior to sensing, the read-out signals obtained were used as baseline (I₀). The nanobody functionalized gate electrode was incubated with 5 μL protein targets solution for 10 min then was washed thoroughly with binding buffer to remove unbounded proteins. The same gate was immersed into the electrolyte on top of the OECT channel to acquire the second transfer curve (I_{D}) at this given concentration. As shown by these transfer curves, OECTs transduce a small change of the input (V_{G}) into large changes of output (I_{D}). The efficiency of the transduction is calculated by the first derivative of the transfer curve, defined as transconductance

$${g}_{m} = \frac{\partial {I}_{D}}{\partial {V}_{G}} .$$

For each sensor, the normalized response (NR) was determined by the normalized change in OECT modulation.

### Nanobody Gate Design HDT Vs SpyDirect

A previous design of the nanobody gate was built on HDT SAM as described in PCT/IB2021/055981, where the maleimide-modified SpyTag peptide is chemically coupled to the HDT SAM to form a combined chemical SAM (chem-SAM) on gold and the nanobody-SpyCatcher fusion protein then attaches itself to this chem-SAM through the autocatalytic formation of a covalent SpyCatcher-SpyTag bond. The design of SpyDirect-biofunctionalization described herein allows sensor surface functionalization in two steps: the 17 amino acid spyTag-peptide (AHIVMVDAYKPTKGSGC) is directly anchored on the gold gate electrode by forming Au—S bonds via the thiol side chain of the C-terminal cysteine, then a spyCatcher-nanobody fusion protein is attached via a self-catalyzing covalent spyTag-spyCatcher coupling (FIG. 1C).

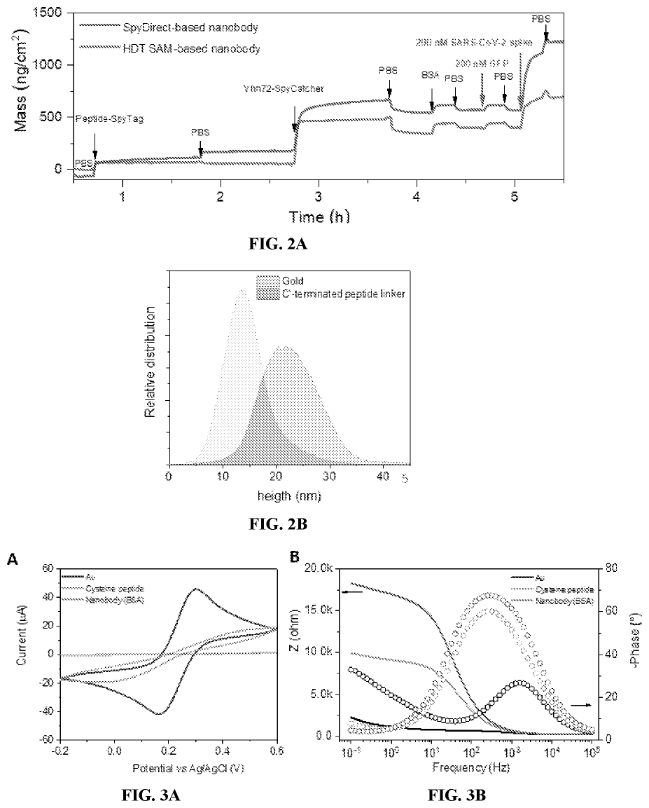

To compare the packing density of biomolecules on the surface, both HDT SAM- and SpyDirect-immobilization steps were monitored on the piezoelectrically active gold sensor by quartz crystal microbalance with dissipation (QCMD) (FIG. 2A, Table 2). The gold QCMD sensors were subjected to either the HDT SAM- or the SpyDirect-based functionalization protocol, respectively. During the biofunctionalization steps, the cumulative mass gain over time as (1) peptide-spy Tag, (2) nanobody-spyCatcher (3) BSA was introduced to the system, followed by washing steps. However, SpyDirect method achieved larger gain of mass in anchoring peptide (241±9 vs 94±32 ng per cm²) and nanobody (376±18 vs 313±26 ng per cm²), respectively. Based on molecular weights of 1.76, 1.78 and 29.05 kDa for the maleimide-peptide, SpyDirect-peptide and SARS-CoV-1 nanobody-SpyCatcher protein, SpyDirect method has the advantage to achieve higher packing density of peptide (82×10¹² SpyDirect-peptide vs 32×10¹² maleimide-peptide per cm²) and nanobody (7.8×10¹² vs 6.5×10¹² per cm²), respectively. The formation of this exceptionally high-density biorecognition layer of SpyDirect-based nanobody surface only allows less BSA (43±26 vs 90±42 ng per cm²) to be absorbed but showing excellent anti-biofouling performance by exposing to a non-target protein (i.e., a green fluorescent protein (GFP) with a concentration of 200 nM). Negligible mass was gained from non-target (less than 1 ng per cm²). However, towards to the real target protein (SARS-CoV-2 spike), SpyDirect-based SARS-CoV-1 nanobody surface has larger binding capability than that of HDT SAM-based surface (659 vs 290 ng per cm²). Hence, the SpyDirect method doubled the binding capability compared to the HDT SAM-based biofunctionalization used in the previous study.

TABLE 2
QCMD summary of biofunctionalization and target protein detection.

| | HDT SAM-based nanobody | | SpyDirect-based nanobody | |
|---|---|---|---|---|
| | gained mass (ng/cm²) | Density per cm² | gained mass (ng/cm²) | Density per cm² |
| Peptide | 94 ± 32 | 3.2 × 10¹ [symbol] | 241 ± 9 | 8.2 × 10¹ [symbol] |
| SARS-CoV-1 nanobody | 313 ± 26 | 6.5 × 10¹² | 376 ± 18 | 7.8 × 10¹² |
| BSA | 90 ± 42 | — | 43 ± 26 | — |
| Non-target GFP (200 nM) | <4 | — | <1 | — |
| 200 nM SARS- CoV-2 spike | 290 | 2.3 × 10¹² | 659 | 5.2 × 10¹² |

[symbol]

indicates data missing or illegible when filed

### Atomic Force Microscopy

Atomic force microscopy (AFM) confirmed the change of the surface roughness and feature height of the biofunctionalized gate in its the wet state (10 mM PBS, pH 7.4) (FIG. 2B). Compared to the bare gold, the nanobody/BSA modified gold gate increased 1.5 nm of the root mean square (RMS) roughness, and 8.4 nm of the average feature height.

### Biofunctionalization of SpyDirect Gate

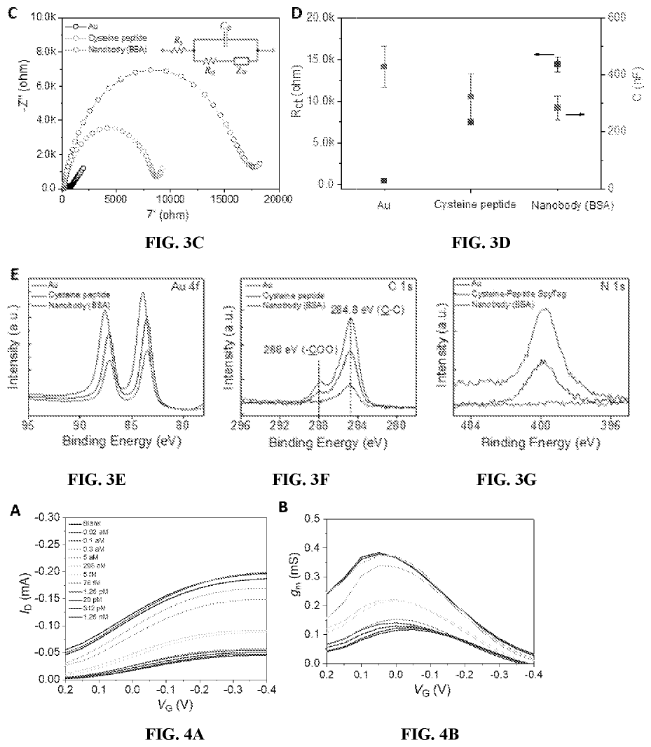

The biofunctionalization of the gate electrodes was assessed biophysically. Firstly, CV and EIS were used to monitor the surface modification process through observing the changes of the gate electrode after each assembly step. As shown by the CV (FIG. 3A), the bare Au electrode presents the expected reversible peaks for the [Fe(CN)₆]^{3-/4-} redox couple with a reduction peak current of 42 μA, an oxidation peak current of 44 μA, and a peak potential separation of 150 mV. After adding the peptide layer, the electron transfer from the redox probes to the gold is inhibited, confirming that a continuous peptide layer had formed on the gate electrode. The permeability of ions through the peptide layer was drastically reduced and the corresponding current response decreased accordingly. When the nanobody was immobilized via spyTag-spyCatcher coupling chemistry, the penetration of the redox probe was further reduced, decreasing the current to almost zero. Additionally, EIS was used to characterize these subsequent modifications by plotting the representative Bode and Nyquist plots of the Au gate electrode after each step (FIGS. 3B and 3C). A Randles equivalent circuit was used to quantitatively analyze the impedance spectra (inset of FIG. 3C). It includes the electrolyte resistance (Rₛ), electric double-layer capacitance (C_{dl}) formed at the electrode/electrolyte interface, charge transfer resistance (R_{ct}) of the electrode, and Warburg impedance resulting from the diffusion of ions from the electrolyte to the electrode surface. After anchoring the spyTag peptide on the electrode, the diameter of the semicircle in the high frequency region of the Nyquist trace increased significantly, indicating an increased impedance. Modeling the Nyquist plots showed that the R_{ct} increased from 0.5 to 7.5 kΩ with peptide addition, confirming the charge blocking behavior of the peptide layer (FIG. 3D). After further immobilization of spyCatcher/nanobody (with BSA blocker), the impedance further increases, and the Nyquist plot becomes a large semicircle that extends across the entire range of frequencies (R_{ct}≈14.4 kΩ). These measurements showed that biofunctional layers on gold increased the charge transfer resistance and reduced the capacitance, demonstrating the successful biofunctionalization in each step. The successful immobilization of nanobodies on the gate surface was also confirmed by XPS (FIGS. 3E-3G). Concomitant with the adding of subsequent layers of peptide and nanobody onto the gate surface, both N1s and C1s XPS spectra increased in intensity, while the intensity of the Au 4f spectrum decreased, due to coverage of the gold surface.

### Detection of SARS-CoV-2 Using SpyDirect Nanobody Gate

To assess the performance of the SpyDirect sensor, the well-characterized GFP-recognizing nanobodies was used. First, a baseline was recorded using a GFP nanobody-functionalized gate after incubation with PB (40 mM, pH 7.4). The same gate was then incubated with increasing concentrations of GFP proteins for 10 min, and thoroughly washed. The corresponding transfer curves were recorded (data not shown). The transfer curves for increasing GFP concentrations decreased in current and showed a significant shift in the threshold voltage (Vₜₕ) towards more negative values. The ID trend can be attributed to the specific interaction between the GFP nanobodies and GFP, but not the OECT instability, because the OECT devices perform stably over a long operation period (data not shown). Conversely, no significant change of the transfer characteristics was observed when the GFP nanobody functionalized gate electrode was incubated with solutions containing 20 nM to 325 nM of lysozyme (data not shown). Lysozyme was chosen as the negative target because it is abundant in saliva (salivary lysozyme concentration is 154 nM), which was intended to use as the medium for the SARS-CoV-2 application. The maximum value of gₘ for the blank sensors is obtained at V_{D}=−0.1 V, V_{G}=−0.05 V, which is chosen to determine the sensor calibration curves and to maximize the sensor performance. The normalized response (NR) was calculated to measure the sensor performance. The sensor responded significantly to GFP concentrations as low as 1.2 aM (NR=20%) and displayed a dynamic range spanning 10 orders of magnitude (FIG. 3C). The lowest limit of detection (LOD) was calculated for these devices to be 5.5×10⁻¹⁹ M, corresponding to 1˜2 GFP molecules in the 5 μL sample. The sensor response to non-target protein (lysozyme with a concentration of 325 nM) was negligible (NR<10%). The specificity of the sensor is independent on the gate voltage (data not shown).

Having validated the sensitivity and specificity of the GFP OECT sensors with target proteins solubilised in buffer, the sensor's performance was tested in unprocessed human saliva to which GFP was added. The NR increased with increasing concentrations of GFP in saliva in a similar manner to the measurements performed with GPF in buffer, especially in the low target concentration range (data not shown). The sensitivity for GFP in saliva (LOD=7.4×10⁻¹⁹ M) was comparable to that in buffer (LOD=5.5×10⁻¹⁹ M) indicating that the complexity of the saliva has negligible effects on the sensitivity and selectivity of the nanobody sensor.

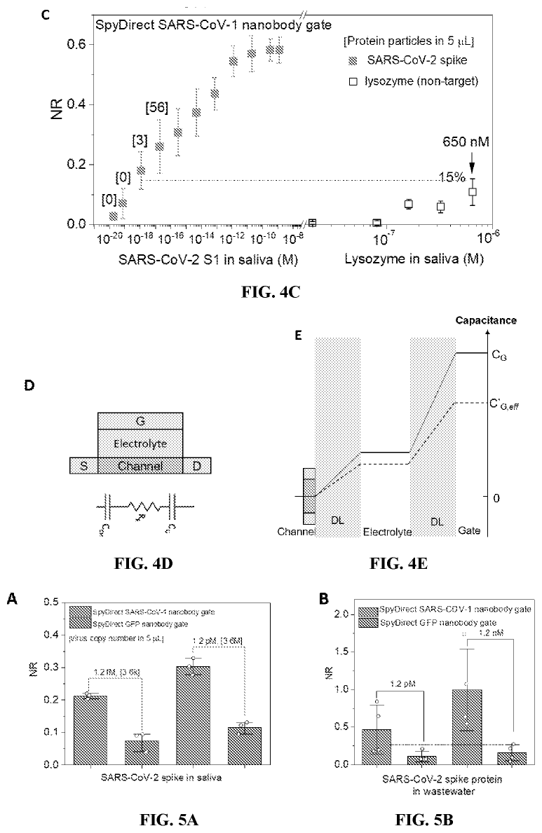

Having demonstrated that the SpyDirect OECT sensors can selectively detect GFP both in buffer and saliva with ultra-high sensitivity (data not shown), the OECT sensors were modified for COVID-19 diagnostics by replacing the GFP nanobody with a nanobody (VHH72) that recognizes the receptor binding domain (RBD) of spike proteins from both SARS-CoV-1 and SARS-CoV-2 with similar affinity. First, a baseline was recorded using a SARS-CoV-1 nanobody-functionalized gate after incubation with phosphate buffer (PB, 40 mM, pH 7.4). The same gate was then incubated with increasing concentrations of SARS-CoV-2 spike protein for 10 min, and thoroughly washed. The corresponding transfer curves were recorded (FIG. 4A). The transfer curves for increasing SARS-CoV-2 spike concentrations decreased in current and showed a significant shift in the threshold voltage (Vth) towards more negative values. The ID trend can be attributed to the specific interaction between the SARS-CoV-1 nanobodies and SARS-CoV-2 spike protein, but not the OECT instability, because the OECT devices performed stably over a long operation period (data not shown). Conversely, no significant change of the transfer characteristics was observed when the SARS-CoV-1 nanobody functionalized gate electrode was incubated with solutions containing 20 nM to 650 nM of lysozyme (data not shown). Lysozyme was used as the negative target because it is abundant in saliva (salivary lysozyme concentration is 154 nM), which was intended to use as the medium for the SARS-CoV-2 application. The maximum value of gₘ for the blank sensors is obtained at V_{D}=−0.1 V, V_{G}=0.05 V, which is chosen to determine the sensor calibration curves and to maximize the sensor performance (FIG. 4B). gₘ decreases upon more protein binding as shown in FIG. 4B. The normalized response (NR) was calculated to evaluate the sensor performance. The sensor responded significantly to SARS-CoV-2 spike concentrations as low as 1.2 aM (NR=20%) (FIG. 4C). This concentration equals 3 protein particles in 5 μL sample solution, showing that the OECT sensors are capable of reaching single molecule sensitivity. Apart from the high sensitivity (LOD=6×10⁻²² M), the SARS-CoV-2 OECT sensors achieved a dynamic range of 10 orders of magnitude in this complex biological medium. In contrast, the OECT sensors showed only a negligible response (NR<15%) to the non-target (20-650 nM lysozyme) in saliva (FIG. 4C).

In an OECT, two interfaces are considered in the gate-electrolyte-channel structure (FIG. 4D), gate/electrolyte interface and channel/electrolyte interface. According to the Bernard model, a purely capacitive process of the ionic circuit is involved in OECT operation. Due to the insulating properties of these protein target molecules, the capacitance of the gate electrode decreases upon the binding events (data not shown). The applied gate voltage is distributed at two interfaces, depending on the magnitude of the capacitance at each surface. The voltage drop at the gate electrode and the channel is described by the following equations:

$${V}_{CH} = \frac{\frac{1}{j ω {C}_{CH}}}{\frac{1}{j ω {C}_{G}} + \frac{1}{j ω {C}_{CH}} + {R}_{E}} \qquad ( 1 )$$

where, V_{CH} is the voltage drop at the channel surface, j is the complex number, ω is the angular frequency, C_{CH} is the channel capacitance C_{Q} is the gate capacitance and R_{g} is the resistance of the electrolyte. In the DC regime, the equation above can be simplified to:

$${V}_{CH} = \frac{1}{1 + \frac{{C}_{CH}}{{C}_{G}}} \qquad ( 2 )$$

So, the corresponding voltage drop V_{G} at the gate electrode can be described as:

$${V}_{G} = 1 - {V}_{CH} \qquad ( 3 )$$

As target protein molecules bind onto the gate electrode, C_{G} decreases to C′_{G,eff}, which leads to a larger proportion of the applied V_{G} to drop at the gate/electrolyte interface (FIG. 4E). Thus, the nanobody-protein bindings at the gate electrode result in a less pronounced doping capability, thus a less efficient transistor operation, and a decrease drain current was observed.

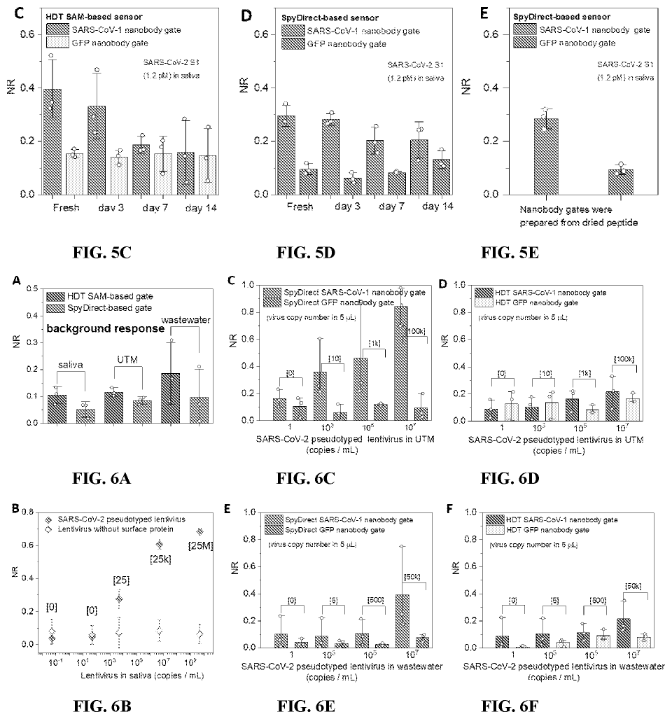

The high sensitivity and selectivity is further evident by random detection of SARS-CoV-2 spike in saliva with a concentration of 1.2 fM and 1.2 pM, respectively (FIG. 5A). SpyDirect gates functionalized with either SARS-CoV-1 nanobody or GFP nanobody were incubated with 5 μL SARS-CoV-2 spike in saliva, SARS-CoV-1 nanobody gates responded with larger NR than that of the GFP nanobody gates to SARS-CoV-2 spike. The SpyDirect nanobody gates were further challenged to detect SARS-CoV-2 spike from untreated wastewater. The SARS-CoV-2 spike with random concentration of 1.2 pM and 1.2 nM in wastewater were differentiated by SpyDirect SARS-CoV-1 nanobody gates using GFP nanobody gates as control, respectively (FIG. 5B). The SpyDirect SARS-CoV-1 nanobody gates showed improved long-term stability (FIGS. 5C and 5D). Compared to freshly prepared SARS-CoV-1 nanobody gates, the response of HDT SAM-based SARS-CoV-1 nanobody gates to 1.2 pM SARS-CoV-2 spike protein in saliva dropped to 92% response after a 3-day storage in PBS buffer; after 7-day storage, these gates could not differentiate anymore SARS-CoV-2 spike from lysozyme due to the similar NR. Conversely,

SpyDirect gates preserved 67% response after 7-day storage, and were still capable of discerning the spike protein with a concentration of 1.2 pM (NR=20%) from lysozyme (NR<12%). In addition, the simplified biofunctionalization of SpyDirect OECT sensors also improved the ease of sensor use and storage, because peptide-coated gate electrodes can be stored for a certain period under ambient conditions without losing its function, before nanobody immobilization. SpyDirect OECT SARS-CoV-1 nanobody sensors stored for seven days after SpyDirect-peptide fixation could be fully functionalized and effectively detect SARS-CoV-2 spike protein target (FIG. 5E). A similar NR was obtained as compared to freshly prepared sensors.

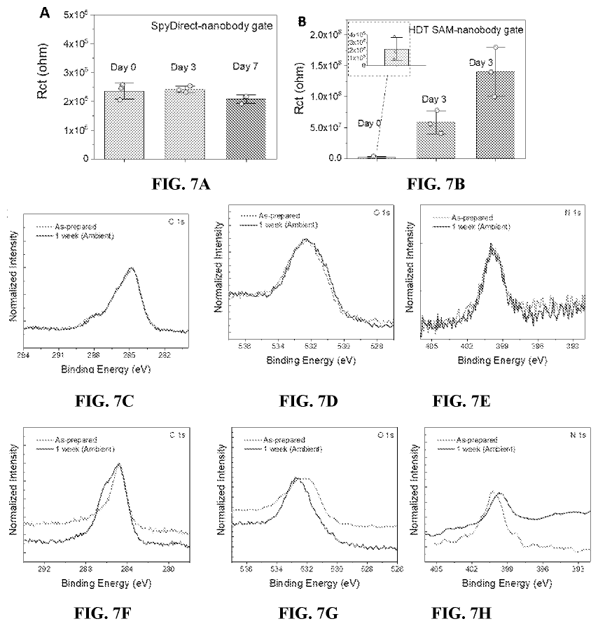

The HDT SAM-based OECT sensors in previous report achieved single molecule sensitivity with a LOD of 1.3×10⁻²¹ M in unprocessed saliva. In contrast, the SpyDirect OECT sensors showed a lower background noise level than the HDT SAM-based sensors after exposing the SARS-CoV-1 nanobody gates in raw saliva, universal transport medium (UTM) and untreated wastewater (FIG. 6A). The lower background noise level in raw saliva makes the SpyDirect OECT sensor more sensitive than its HDT SAM counterparts in saliva (LOD 6×10⁻²² vs 1.3×10⁻²¹ M, respectively).

Finally, as a proxy to viral loads in patient samples, the SpyDirect sensors were tested against SARS-CoV-2 recombinant lentivirus pseudotyped with spike protein in human saliva, UTM and untreated wastewater. The OECT sensor showed specific response to the SARS-CoV-2 pseudotyped lentivirus, but not the negative control in saliva (FIG. 6B). The NR increased with the copy number of the SARS-CoV-2 pseudotyped lentivirus, but showed only negligible change when the same gate electrode was exposed to the equivalent copy numbers of virus particles that did not display SARS-CoV-2 spike proteins. The sensors can detect 25 copies of the virus particles in a 5 μL sample volume (average NR change was 28±6%), which is larger than the background noise from the negative controls (16%). The LOD of the sensor for the SARS-CoV-2 pseudotyped lentivirus in saliva is 200 copies per mL. Furthermore, SpyDirect-SARS-CoV-1 nanobody sensors showed superior detection capability in response to SARS-CoV-2 pseudotyped lentivirus in both UTM and wastewater. Using GFP nanobody gates as controls, SpyDirect SARS-CoV-1 nanobody sensors can reliably detect SARS-CoV-2 pseudotyped lentivirus as little as 10 copies in UTM (FIG. 6C vs FIG. 6D, HDT SAM-sensor 1000 copies) and 500 copies in wastewater (FIG. 6E vs FIG. 6F, HDT SAM-sensor 50000 copies), respectively.

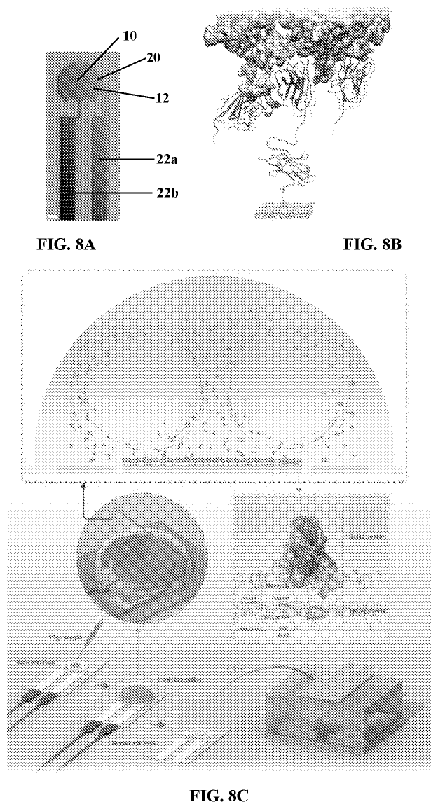

The excellent sensor performance of SpyDirect gates may be contributed to their superior electrochemical and chemical stability. Compared to fresh gates (237 k ohm), the Rct average value of the SpyDirect nanobody gates showed negligible change after 3-day storage in PBS (242 k ohm), while a decrease of 12.5% after 7 days' storage (FIG. 7A). In contrast, the HDT SAM-based nanobody gates showed a dramatic increase of the average Rct values after storage (26-fold increase after 3-day storage, 64-fold increase after 7-day storage, respectively) (FIG. 7B). The XPS further confirmed the negligible change in chemical structures of C1s, O1s and N1s of the SpyDirect-based nanobody gates after long-term storage (FIGS. 7C-7E). However, shifting in XPS spectra of C1s, O1s and N1s were observed in the case of HDT SAM-based nanobody surface after 1-week storage in PBS (FIGS. 7F-7H).

The poor chemical stability of the HDT SAM may be mainly caused by the instability of HDT SAM-base nanobody surface. As demonstrated in previous study, taking advantage of the autocatalytic attachment of spyCatcher-nanobody fusion proteins, the Bio-SAM layer (peptide-spy Tag/SpyCatcher-nanobody) possess high flexibility ranging from 12 to 20 nm in wet. Once the HDT SAM was attacked, the Bio-SAM layer collapses and randomly covers the gold electrode forming extra insulating layers. This further hinders the diffusion of redox probes to the electrode surface due to the loss of flexibility, a dramatic increase in Rct was observed. Due to the fact that a new surface was exposed over time, the shifting in XPS spectra happened accordingly.

### Discussion

Conventional biofunctionalization methods use thiol SAM as a linker to build immunosensors. However, this strategy results in low sensor stability when used under ambient or biological conditions. The SpyDirect described herein is a simplified biofunctionalization method for OECT immunosensors. SpyDirect uses cysteine-terminated spyTag peptides to directly link nanobodies to the gold surface of the gate electrode. The autocatalytic spyCatcher-nanobody module was designed to improve nanobody orientation and packing. This strategy produces sensors that display flexible ultra-high-density arrays of productively oriented nanobody bioreceptors. Owing to these features, the SpyDirect OECT sensors can detect less than 200 copies of SARS-CoV-2 pseudotyped lentivirus from raw saliva, UTM, and untreated wastewater in less than 15 min. Thus, the sensor achieves a LOD of 6×10⁻²² M and covers a dynamic range of more than 10 orders of magnitude. Compared to the conventional biofunctionalization method using thiol SAM, SpyDirect biofunctionalization showed lower background noise, improved long-term stability, as well as easier fabrication, storage, and use. Hence, SpyDirect biofunctionalization represents an improved method for next-generation sensing devices including but not limited to immunosensors.

### Example 2. Convection Driven Ultra-Rapid Detection

### Materials and Methods

### Materials

Sodium chloride, Tween-20, glycerol, HEPES, bovine serum albumin (BSA), (3-glycidyloxypropyl) trimethoxysilane (GOPS), ethylene glycol (EG), 1,6-hexanedithiol (HDT), 3-mercaptopropionic acid (MPA), and PBS (pH 7.4) were purchased from Sigma Aldrich and used without processing. Solutions were prepared with ultrapure water (Millipore Milli-Q). p(C6NDI-T) was synthesized according to a procedure reported previously. The synthetic route for p(g₃C₂T2-T) is provided below. Materials for protein purification are as follows: Agar, LB broth, 2×YT Broth, kanamycin, glucose, isopropyl β-d-1-thiogalactopyranoside (IPTG), BugBuster (Novagen), complete protease inhibitor mix (Sigma), Benzonase (Novagen), Egg-Lysozyme (Fluka), tris(2 carboxyethyl) phosphine (TCEP), tris(hydroxymethyl)aminomethane hydrochloride (Tris-HCl), imidazole, glycerol, dithiothreitol (DTT), ethylenediaminetetraacetic acid (EDTA), d-desthiobiotin, 10 K Amicon ultra spin concentrators (Milipore). Viral target proteins were purchased from Sino Biological: SARS-CoV-1 RBD (40150-V08B2), SARS-CoV-2 RBD (40592-V08H), and SARS-CoV-2 S1 (40591-V08B1).

Synthesis and Characterization of p(g₃C₂T2-T)

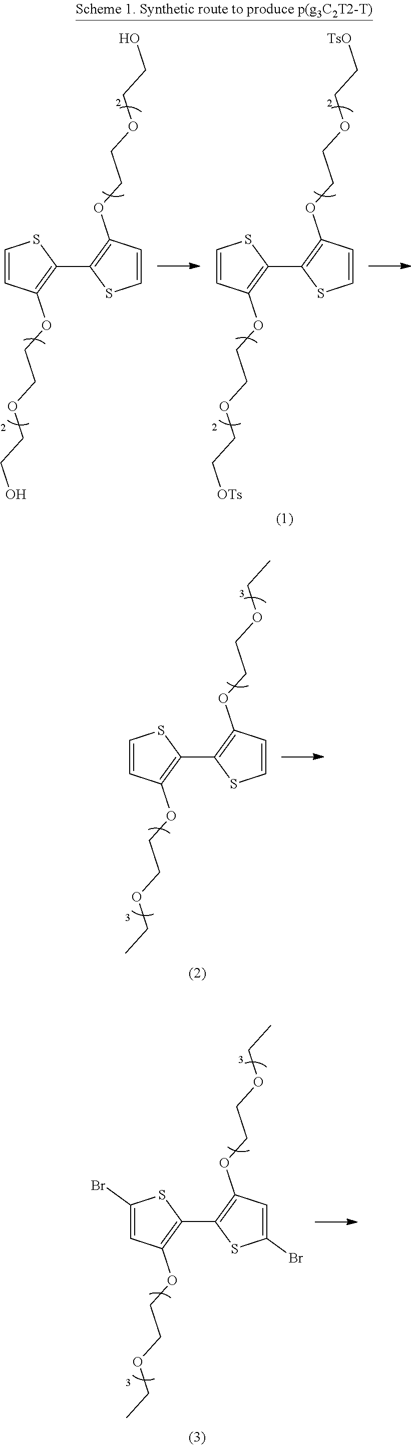

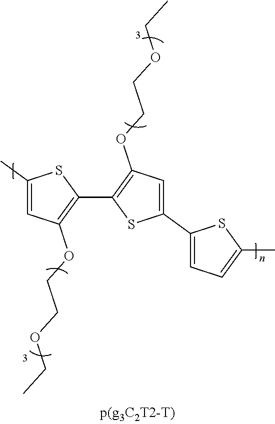

### ((((([2,2′-bithiophene]-3,3′-diylbis(oxy))bis(ethane-2,1-diyl))bis(oxy))bis(ethane-2,1-diyl))bis(oxy))bis(ethane-2,1-diyl) bis(4-methylbenzenesulfonate) (1)

2,2′-((((([2,2′-bithiophene]-3,3′-diylbis(oxy))bis(ethane-2,1-diyl))bis(oxy))bis(ethane-2,1-diyl))bis(oxy))bis(ethan-1-ol)1 (11.63 g, 25.1 mmol, 1.00 eq.) was dissolved in 30 mL of pyridine and cooled to 0° C. 4-Toluenesulfonyl chloride (11.51 g, 5.25 mmol, 2.4 eq.) was added portion wise. The solution was stirred for 30 minutes before being left overnight in the refrigerator. Upon completion, 200 mL of water was added, followed by 100 mL of 2M HCl, the aqueous phase was extracted three times with dichloromethane. The combined organic layers were then washed with water, before being dried over sodium sulfate. Excess solvent was removed under reduced pressure to afford the crude product, as a colorless oil (17.81 g, 1.30 mmol, 92% yield) which was used without further purification. 1H NMR (400 MHZ, CDCl3) δ 7.82-7.72 (m, 4H), 7.37-7.27 (m, 4H), 7.06 (d, J=5.6 Hz, 2H), 6.83 (d, J=5.5 Hz, 2H), 4.27-4.15 (m, 4H), 4.17-4.06 (m, 8H), 3.77-3.55 (m, 12H), 2.40 (s, 6H).

### 3,3′-bis(2-(2-(2-ethoxyethoxy)ethoxy)ethoxy)-2,2′-bithiophene (2)

Sodium hydride (3.36 g, 84 mmol, 10.50 eq.) was dissolved in 200 mL anhydrous tetrahydrofuran and cooled to 0° C. Anhydrous ethanol (4.67 mL, 79.9 mmol, 10.00 eq.) was then added dropwise. Following this, the reaction mixture was stirred at 65° C. for 60 minutes. The reaction mixture was then cooled to 0° C., next a solution of compound 1 (6.17 g, 8.00 mmol, 160 mmol, 1.00 eq.) in 100 mL tetrahydrofuran was added dropwise. The mixture was stirred overnight at 55° C. Water was added and the aqueous phase was extracted three times with dichloromethane. The combined organic layers were washed with water and brine before being dried over sodium sulfate. Excess solvent was removed under reduced pressure. The final product was obtained by silica column chromatography eluting with ethyl acetate as an off white solid (1.70 g, 3.28 mmol, 41% yield). 1H NMR (400 MHZ, CDCl3) δ 7.06 (dd, J=5.6, 0.9 Hz, 2H), 6.84 (dd, J=5.6, 0.9 Hz, 2H), 4.27-4.20 (m, 4H), 3.89 (dd, J=5.6, 4.5 Hz, 4H), 3.78-3.70 (m, 4H), 3.69-3.52 (m, 12H), 3.59-3.43 (m, 4H), 1.19 (t, J=6.9 Hz, 6H). 13C NMR (101 MHz, CDCl3) δ 151.85, 122.01, 116.69, 114.90, 71.50, 71.05, 70.83, 70.81, 70.79, 70.72, 70.13, 69.94, 66.73, 15.27.

### 5,5′-dibromo-3,3′-bis(2-(2-(2-ethoxyethoxy)ethoxy)ethoxy)-2,2′-bithiophene (3)

Compound 2 (0.86 g, 1.66 mmol, 1.00 eq.) was dissolved in 100 mL anhydrous tetrahydrofuran. The resulting solution was degassed for 15 minutes and cooled to −20° C. N-bromosuccinimide (0.62 g, 3.48 mmol, 2.10 eq.) was then added portion wise in the dark. The progress of the reaction was monitored by thin layer chromatography employing ethyl acetate as the eluent. Once the reaction was complete, after approximately 60 minutes, the reaction mixture was poured into a saturated aqueous sodium bicarbonate solution. The aqueous phase was extracted three times with ethyl acetate and the combined organic layers washed with water and brine prior to being dried over sodium sulfate. Excess solvent was removed under reduced pressure. The final product was obtained following column chromatography on silica employing ethyl acetate as the eluent, as a white solid (0.92 g, 1.36 mmol, 82% yield). 1H NMR (400 MHZ, CDCl3) δ 6.86 (s, 2H), 3.86 (dd, J=5.7, 4.0 Hz, 4H), 3.77-3.62 (m, 16H), 3.59 (dd, J=6.1, 3.6 Hz, 4H), 3.52 (q, J=7.0 Hz, 4H), 1.20 (t, J=7.1 Hz, 6H). 13C NMR (101 MHz, CDCl3) δ 151.87, 122.03, 116.71, 114.91, 72.09, 71.52, 71.06, 70.85, 70.76, 70.71, 70.16, 59.16.

### Poly-3,3′-bis(2-ethoxyethoxy)-5,5″-dimethyl-2,2′:5′,2″-terthiophene (p(g3C2T2-T))

Compound 3 (180 mg, 0.266 mmol, 1.00 eq.), 2,5-bis(trimethylstannyl)thiophene (109 mg, 0.267 mmol, 1.00 eq.) and tetrakis(triphenylphosphine)palladium(0) (6.2 mg, 0.053 mmol, 0.020 eq.) were added to a 10 mL microwave vial, subsequently 1.8 mL of anhydrous dimethylformamide and 1.8 mL of anhydrous chlorobenzene was added. The vial was sealed, and the solution was degassed for 10 minutes before being heated to 111° C. overnight. After cooling to room temperature, the reaction mixture was precipitated into methanol. The crude product was purified by sequential Soxhlet extractions in hexane, acetone, ethyl acetate, methanol and finally dissolved into chloroform. Solvent from the chloroform fraction was removed under reduced pressure and the product was reprecipitated into methanol. The final product was collected by suction filtration and was recovered as a dark blue solid (119 mg, 71% yield). GPC (DMF, 40 □C) Mn=174 kDa, [symbol] =1.6, 1H NMR (500 MHz, CDCl3) δ 7.08 (s, 2H), 6.97 (s, 2H), 4.37-4.31 (m, 4H), 4.00-3.94 (m, 4H), 3.81 (dd, J=5.8, 3.7 Hz, 4H), 3.73 (dd, J=6.0, 3.6 Hz, 4H), 3.66 (dd, J=5.9, 3.8 Hz, 4H), 3.57 (dd, J=5.9, 3.8 Hz, 4H), 3.50 (q, J=7.0 Hz, 4H), 1.19 (t, J=7.0 Hz, 6H).

### Fabrication of the OECT and the Gate Electrodes

A standard photolithography protocol was used to fabricate OECTs on glass substrates. First, a layer of photoresists (LOR 5B and S1813) was spincoated on the substrates which were then exposed to the ultraviolet light to pattern the shape of the transistor components. After treating the substrates with a developer (MF-319), 10 nm of chromium (Cr) and 100 nm of gold (Au) were sputtered, and standard lift-off was performed in N-methyl-2-pyrrolidone (NMP) held at 80° C. Two layers of parylene separated by an adhesive were then deposited and a second photolithography step was performed with AZ 10XT as the photoresist to insulate the interconnects and source and drain contacts. After reactive ion etching, the films were spin cast. The second, sacrificial layer of parylene-C was peeled off to pattern the OECT channels with 10 μm of length and 100 μm of width. The concentric gate electrodes were also fabricated on glass substrates using standard photolithography. A first layer of photoresist (AZ2020) was spin-coated and exposed to ultraviolet light using a contact aligner. The photoresist pattern was created by AZ726 developer, and residual photoresist was removed by oxygen plasma. 10 nm Cr and 100 nm Au were deposited by sputtering, followed by lift-off in hot NMP. The area of the concentric gate electrode (25 mm²) was determined by considering the capacitive coupling requirements.

### Biofunctionalization of Gate Electrodes

Before biochemical functionalization, the gold gate electrodes were cleaned by using cyclic voltammetry in an acidic solution. Following a previously reported protocol, the electrodes were connected in a three electrodes setup and immersed in a 10×10⁻³ m H₂SO₄ solution, where 25 cycles from −0.2 to 1.5 V vs Ag/AgCl were applied at a scan rate of 100 mV s⁻¹. The HDT-SAM solution was prepared in 100% ethanol containing 1×10⁻³ m of HDT. The gold electrodes were immersed in this solution for an hour, followed by rinsing in ethanol and dried under nitrogen. The electrodes were then incubated for an hour with the synthetic maleimide-modified Spy Tag peptide solution in PBS (0.1 mg mL⁻¹). After rinsing the electrodes with PBS, they were exposed to the SpyCatcher/Ty1-nanobody fusion proteins at a concentration of 20×10⁻⁶ m in a binding buffer (100×10−3 m HEPES pH 7.4, 150×10−3 m NaCl, 0.05% v/v Tween-20, 0.02% w/v NaN3, 0.1% w/v BSA) for 1 h and then rinsed them once more with PBS. The functionalized gate electrodes were used the same day that they were biofunctionalized.

### MST

The recombinant nanobodies Ty1 and GFP were labeled with the second generation NHS red labeling kit in buffer 2.1 without BSA. Dye in excess was removed by size exclusion chromatography on a Sepharose6 increase column. Fractions were pooled, concentrated, and kept in buffer 2.1 without BSA. Commercial SARS-Cov1 RBD, SARS-Cov2 RBD, and lab-prepared GFP fluorescent protein were not labeled. A 2×serial dilution with unlabeled proteins was analyzed in 16 capillaries with a starting final concentration at 640×10⁻⁹ m. Labeled nanobodies were used at final 2.5×10⁻⁹ m concentration. Binding reactions were incubated at RT for 30 min before analysis. The MST analyses were performed at 20% LED power and HIGH MST power on a Monolith NT.115 pico Instrument (Nano Temper Technologies, Germany). Three replicates were analyzed per binding pair.

### KPFM

KPFM measurements were performed with a Dimension Icon SPM (Bruker) using a SCM-PIT-V2 tip (Bruker). The surface potential reflected by the contact potential difference (VCPD) represents the difference in the work function of the samples and KPFM tips, as shown in the equation below, and can provide insight into the energy band structure of the surfaces:

$${V}_{CPD} = ( {Φ}_{tip} - {Φ}_{sample} ) / ( - e )$$

where Φₜᵢₚ and Φₛₐₘₚₗₑ are the work functions of the tip and sample, and e is the electronic charge.

### XPS

XPS spectra were obtained using a Kratos AXIS Supra instrument equipped with a monochromatic Al Kα X-ray source (1468.6 eV). The source was operated at 75 W under ultrahigh vacuum conditions (≈10−9 mbar). The spectra were recorded in a hybrid mode using electrostatic and magnetic lenses and an aperture slot of 300×700 μm. The high-resolution spectra were acquired at fixed analyzer pass energies of 20 eV. The samples were mounted in a floating mode to avoid differential charging. The spectra were calibrated to a reference of C1s at 284.8 eV. The Tougaard method was used for background subtraction, and Gaussian and Lorentzian methods were used for deconvolution via XPSPEAK41 software.

### SIMS

To characterize the elemental composition of the films, a secondary ion mass spectrometry (SIMS) study was performed. Gold electrodes were spin-coated with the p- and n-type polymers. The films were immersed in 100×10⁻³ m of NaCl solution to allow for passive swelling. A second set of films of each polymer type were immersed in the electrolyte and biased at a doping voltage (|0.5| V vs Ag/AgCl) for 5 min. The films were rapidly removed and inserted into the SIMS setup for analysis. Depth profiling experiments were performed on a Dynamic SIMS instrument from Hiden analytical company (Warrington, UK) operated under ultrahigh vacuum conditions, typically 10−9 Torr. A continuous Ar+ beam of 4 keV energy was used to sputter the surface while the selected ions were sequentially collected using a MAXIM spectrometer equipped with a quadrupole analyzer. The raster of the sputtered area was estimated to be 750×750 μm². To avoid the edge effect during depth profiling experiments, it was necessary to acquire data from a small area located in the middle of the eroded region. Using an adequate electronic gating, the acquisition area from which the depth profiling data were obtained was approximately 75×75 μm².

### QCM-D

QCM-D measurements were carried out using a Q-Sense analyzer (QE401, Biolin Scientific). The piezoelectrically active gold sensor (0.7854 cm2) was pre-coated with 1,6-hexanedithiol (HDT) monolayer and then mounted inside the QCM-D setup. After the stabilization of frequency (f) and dissipation (D) changes of the crystal in PBS, SpyTag peptide solution (0.1 mg mL⁻¹ in PBS) was pumped into the fluidic chamber with a flow rate of 100 μL min⁻¹. After the full coverage of sensor surface with the solution, the pump was stopped and the sensor was let to be covered by the peptide solution. Afterward, the sensor surface was rinsed with PBS flushed through the fluidic for 15 min to remove excess peptides from the surface. The same procedure was conducted to expose the surface of the quartz to the SpyCatcher/Ty1-nanobody fusion proteins (20×10⁻⁶ m, in binding buffer). The seventh overtone was selected to present the QCM-D data. Mass changes (Δm) that the sensor undergoes were calculated using the Sauerbrey Equation:

$$Δ m = [ ( - 17.7 ) / n ] Δ {f}_{n}$$

where n is the overtone number and Δfₙ is the change in QCM-D frequency at that selected overtone.

### EIS

The formation of bio-chemical layers on the gold electrode was examined using EIS. A three-electrode setup was connected to a potentiostat (Autolab PGstat128N with Nova software, MetroOhm). A platinum wire and an Ag/AgCl electrode were used as the counter and reference electrodes, respectively. The gold gate electrode (working electrode) was immersed in 5 mL of PBS (pH 7.4) containing 10×10⁻³ m of [Fe(CN)6]3-/4-. The impedance spectra were measured at a zero DC offset vs the open circuit potential and an AC modulation of 10 mV was applied over a frequency range of 0.1-100 000 Hz. The spectra were analyzed using Nova software and appropriate equivalent circuit modeling.

### Proteins and Peptides

Nanobody-SpyCatcher fusion proteins were designed based on available structures (Ty1 nanobody:6ZXN; SpyCatcher:PDB 4MLI) with the nanobody placed at the N-terminal end of the fusion protein in order to orient the common Ty1 target binding interface toward the bulk solution, away from the sensing surface. Protein sequences were reverse-translated and codon-optimized for expression in E. coli with an in-house Python script based on DNAChise. Plasmids for protein expression were gene synthesized by Twist Bioscience (USA) in the customized expression vector pJE411c with kanamycin resistance and modified with a RBS insulator (BCD2) cassette for improved translation initiation. Plasmids were transformed into E. coli BL21 (DE3) and starter cultures were inoculated overnight from a single colony. 1 L production cultures in 2×YT medium with 50 mg L⁻¹ kanamycin and 1% glucose were inoculated 1:100, grown at 37° C. and 250 rpm to OD600 0.8, induced with 0.5×10⁻³ m IPTG, and incubated shaking for 18 h at 25° C. Cells were harvested by centrifugation for 10 min at 6000 g at 4° C., washed once with cold PBS, resuspended in lysis buffer [25×10⁻³ m Tris-HCl (pH 7.4), 500×10⁻³ m NaCl, 10×10⁻³ m Imidazole, 10% glycerol, complete protease inhibitor (Roche), 25 U mL⁻¹ benzonase HC (Milipore), 2×10⁻³ m DTT], and homogenized with a cell disruptor (Constant Systems, UK). Earlier purifications of GFP nanobody and msfGFP used lysis by sonication. Lysates were cleared by centrifugation at 87000 g for 45 min, the supernatant filtered through Miracloth tissue (Milipore), and subjected to affinity chromatography on an Äkta FPLC (GE Healthcare) using either StrepTrap HP or HisTrap HP columns (GE Healthcare), depending on the purification tag. The Strep-tag binding buffer was 100×10⁻³ m Tris-HCl (pH 8), 150×10⁻³ m NaCl, 1×10⁻³ m EDTA, 5% glycerol, 0.5×10⁻³ m TCEP, and elution was performed with 2.5×10⁻³ m desthiobiotin in binding buffer. The His-tag binding buffer was 25×10⁻³ m Tris-HCl pH 7.4, 500×10⁻³ m NaCl, 10×10⁻³ m Imidazole, 10% glycerol, 2×10⁻³ m DTT, and elution was performed with a four-step imidazole gradient up to 0.5 m. Fractions were pooled and concentrated using 10K Amicon ultra (Milipore) followed by gel filtration on a Superdex75 16/600 column (GE Healthcare) into 20×10⁻³ m HEPES pH 7.5, 300×10⁻³ m NaCl, 10% glycerol, 50×10⁻⁶ m EDTA. After spin-concentration, aliquots were snap-frozen in liquid nitrogen and stored at −80° C. Protein purity, quality, and accurate molar mass were monitored by SDS-PAGE as well as SEC-MALS (size-exclusion chromatography multiangle light scattering) on a Dawn Heleos II & OptiLab T-rEx (Wyatt, USA). Protein concentrations were determined on a Nanodrop spectrophotometer by absorbance at 280 nm using sequence-specific extinction coefficients.

### Fluorescence Microscopy

Imaging was performed on a DMI8 inverted fluorescence microscope (Leica Microsystems) coupled with a pE-4000 fluorescence illumination system (CoolLED), and the images were processed using ImageJ software. The presence protein was studied by monitoring the binding of GFP and mCherry to the GFP nanobody gate electrode solution after ACET-facilitated incubation. The green and red emitted lights from the electrode surface were monitored.

### Protein Dilutions

All proteins were thawed on ice and centrifuged at 15000 rpm at 4° C. for 30 to 45 min in order to remove any potential aggregates (although no aggregation was observed). Protein dilutions were prepared in the sensor binding buffer (20×10⁻³ m HEPES pH 7.4, 150×10−3 m NaCl, 0.05% v/v Tween-20, 0.02% w/v NaN3, 0.1% w/v BSA). Saliva spike-in measurements used a modified lysis buffer (20×10⁻³ m HEPES pH 7.4, 500×10⁻³ m NaCl, 1% v/v Triton-X, 0.02% w/v NaN3, 0.1% w/v BSA) supplemented with Complete protease inhibitor cocktail with EDTA (Sigma) at four times the manufacturer recommended concentration. It was verified that dilutions of the Sino Biologicals storage buffer by itself did not give any sensor response. Weak background sensor signals were recorded from dilutions of DTT. In-house proteins were therefore stored or exchanged into DTT-free buffer before use. The higher-concentrated proteins from in-house production were first diluted to intermediate concentrations that could still be validated and corrected spectrophotometrically. Four fold dilution series were prepared in 96-well microplates over 23 steps starting from 320×10⁻⁹ m. For measurements in saliva, dilutions were mixed 3:1 with saliva before the measurement (3 volumes protein dilution, 1 volume saliva). Saliva samples were self-collected in the morning before food or tooth brushing by healthy volunteers as part of registered protocols approved by King Abdullah University of Science and Technology (KAUST) Institutional Biosafety and Bioethics Committee (IBEC) (under project numbers 18IBEC11 and 20IBEC25). All volunteers provided signed consent to participate in the study.

### OECT Characterization and Sensor Operation

The steady-state characteristics of the transistor were recorded using a Keithley 2602A type source meter unit operated by a customized LabVIEW software. The drain (VD) and gate (VG) voltages were applied while the source electrode was the common ground. A PDMS well (1 cm diameter, 2 mm thick) was placed on top of the OECT and filled with 100 μL of PBS (pH 7.4, ionic strength 0.162 m). The p(g3C2T2-T) OECT was operated by varying the VG between 0.2 and −0.6 V while the VD was swept from 0 to −0.6 V. For the p(C6NDI-T) OECT, VD was swept from 0 to 0.6 V while VG was varied from 0 to 0.9 V. The ID and gate current (IG) were simultaneously monitored. After functionalization steps, the reference (blank) response of the sensor was obtained by immersing the gate electrode in the PBS. The same electrode was then incubated with the sample (10 μL) for 2 min under the ACET flow, rinsed in buffer 2.1 and twice in PBS, and mounted on top of the channel in a parallel fashion to complete the OECT biosensor. To quantify the sensor response and minimize device-to-device variations, the normalized response (NR) of the OECT was calculated by normalizing the protein-induced change in ID at a single VD and VG to its value measured after exposure to the blank solution (I0):

NR=|ID−I0|/I0

where ID is the current response of the sensor to an analyte solution that the gate was exposed to. After each sensing measurement, the channel response gated with Ag/AgCl was recorded.

### Results and Discussion

### The Design of the Nanobody-OECT Protein Sensor

The OECT has two main components: the organic semiconductor film in the channel, that is, the p-type p(g₃C₂T2-T) or the n-type p(C₆NDI-T) (width of 100 μm, length of 10 μm, and thickness of ≈85 nm) and the nanobody-functionalized gold gate electrode (25 mm²) (FIG. 8A). The structures of p-type p(g₃C₂T2-T) or the n-type p(C₆NDI-T) are shown below.

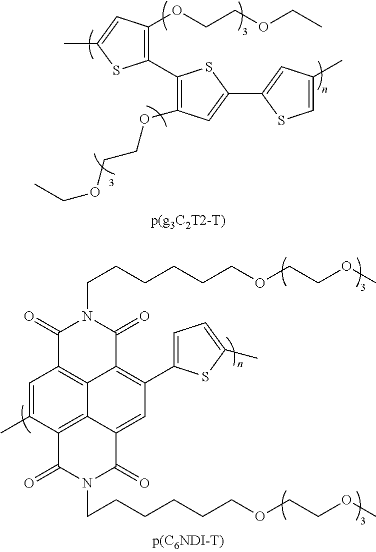

Both of these polymers are mixed, ionic and electronic charge conductors. Their films contain traces of their respective dopant ions throughout their bulk after electrochemical doping, revealed using ex situ secondary ion mass spectroscopy (SIMS) (data not shown), and the capacitance scales with film thickness (data not shown). For the p(g3C2T2-T) channel, a negative gate voltage (VG) pushes anions into the film which compensates for the holes injected from the metal contact, turning the device on with a threshold voltage (Vth) of −0.13 V (data not shown). The maximum transconductance, which quantifies the maximum change in the drain current (ID) with a gate potential modulation (gm=∂ID/∂VG), is −60 mS at VG=−0.35 V at the saturation regime (VD=−0.35 V). For the p(C6NDI-T) OECT, a positive VG drives the cation-electron coupling in the channel, switches the current above a Vth of 0.27 V with a gm of ≈15 μS (VG=0.5 V, VD=0.5 V) (data not shown). Both OECTs have an on/off ratio of ≈515 under these operating conditions.

The operational stability of these channels was evaluated by monitoring the ID for 1 h during the application of square shared voltage pulses at the gate electrode with an amplitude of 0.5 V and duty cycle of 10 s. Among identical devices made of a selection of other organic semiconductors commonly used for OECTs, these devices were among the most stable, with p(g3C2T2-T) retaining 99% and p(C6NDI-T) 95% of its original channel current after 1 h (data not shown).

FIG. 1B schematically illustrates the biological construct on the gate electrode. The biorecognition unit of the sensor is the recognition domain of a single-chain antibody, known as nanobody. The nanobody is recombinantly produced as a fusion protein linked through a flexible linker to a SpyCatcher domain. The SpyCatcher domain autocatalytically covalently binds to a commercially synthesized SpyTag peptide that is chemically immobilized on a 1,6-hexanedithiol (HDT) based monolayer assembled on top of the gold electrode. This indirect functionalization strategy leads to the immobilization of the nanobody on the electronics surface via the HDT. An alpaca-derived nanobody Ty1 that specifically recognizes the receptor binding domain (RBD) of the SARS-CoV-2 spike glycoprotein was used. The affinity and specificity of this nanobody was evaluated using microscale thermophoresis (MST) analyses, where it showed a strong affinity to SARS-CoV-2 RBD (KD=24.9±10.3×10⁻⁹ m) and minimal interactions with SARS-CoV-1 RBD or green fluorescent protein (GFP) (data not shown).

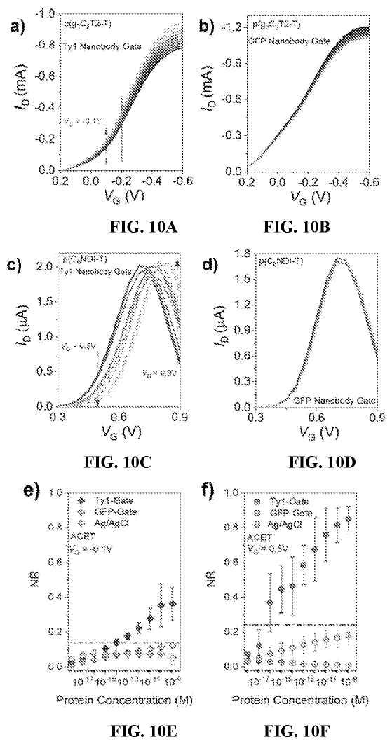

To characterize the gold electrode surfaces and monitor their functionalization with the HDT, SpyTag peptide, and nanobody-spyCatcher fusion, a combination of techniques was used, namely, electrochemical impedance spectroscopy (EIS), quartz crystal microbalance and dissipation monitoring (QCMD-D), Kelvin probe atomic force microscopy (KPFM), and X-ray photoelectron spectroscopy (XPS) (FIGS. 9A-9F). FIGS. 9A-9C present the representative Nyquist plots of the gold gate electrode after each functionalization step. As each (bio) chemical layer was formed on the surface, the semicircle of the Nyquist trace got larger. Using an equivalent circuit fit, these changes were shown as an increase in the system's charge transfer resistance (from 0.14 kΩ to 4.83 kΩ) and a decrease in its double layer capacitance (from 3.26 to 1.12 μF) as the nanobody layer covers the gold electrode surface. The topography images acquired via KPFM measurements showed that the chemical layer and the bioreceptors made the gold electrode surface slightly rougher than its pristine form (root mean square roughness (RMS)=1.57 nm vs 1.72 nm for Au vs Au/HDT/nanobody construct). The nanobody-functionalized electrode surface seemed relatively homogeneous without any large aggregates, showing the presence of uniformly immobilized species (data not shown). Using KPFM, the contact potential difference of the gold electrode was measured and found that it increased with the attachment of each functional layer (VAu=21.57 mV, VHDT=60.61 mV, and Vnanobody=194.41 mV). These results indicate a decrease in the function of the gold electrode as the insulating chemical SAM and protein layers reduce the electron injection efficiency.

QCM-D allows monitoring the formation of these layers in real-time. FIG. 9D shows that the change in the frequency of the crystal's oscillations increased upon injection of the SpyTag peptide and Ty1-SpyCatcher on the HDT-coated gold surface (top panel). These changes correspond to a mass density increase of 68 ng cm⁻² after the SpyTag peptide binds to the HDT layer (bottom panel). After the introduction of the Ty1-nanobody-SpyCatcher protein, a mass density increase of 264 ng cm⁻², corresponding to 5.7×10¹² nanobodies per square centimeter of gold was observed. XPS confirmed the presence of the HDT layer and the bio-layers on the gold surface (FIG. 9E). The high-resolution C1s spectrum of HDT-functionalized gold exhibited two deconvoluted peaks. The main one was attributed to the six-carbon chain of the HDT molecule (C—C at 284.7 eV). A smaller peak appeared at 286.2 eV, corresponding to the C—O bond. This peak was attributed to residual ethanol used during the functionalization and rinsing steps. Upon immobilization of the Spy Tag peptide, new peaks appeared in the C1s spectrum at 285.2 eV and 288.5 eV, corresponding to the C—N and C—OOR bonds, respectively, that originate from the peptide amino acids. Conversely, the high-resolution N1s spectra of the HDT-functionalized gold surface were featureless (FIG. 9F). For the peptide-functionalized electrode, the nitrogen peak was observed at ≈400 eV, with an increase in its intensity occurring after the nanobody-functionalization. The presence of the nanobody-SpyCatcher on the surface was corroborated by the increased signal of the C—O bonds in the overall C1s spectrum. Collectively, these findings showed that the functional layer is established on top of the gold electrode, as illustrated in FIG. 8B.

### The ACET Integrated Sample Incubation

A concentric gate electrode design (FIG. 8A) allows application of an AC potential through the solution to accumulate the target molecules (such as the Spike protein) on the immobilized nanobodies. The non-uniform AC electrical field increases the local temperature because of the Joule heating effect. The local temperature rise generates gradients in the density, permittivity, and conductivity of the solution. As a result of the combined effect of these gradients and the applied electrical field, an electrohydrodynamic force is exerted on the fluid that stirs the solute molecules by the induced flow. However, ACET-induced self-heating must be evaluated, especially in high ionic strength solutions such as PBS (pH 7.4, ionic strength 0.162 m), to ensure that ACET does not create excessive heat and is safe to use in biological media. To estimate the magnitude of the rise in temperature due to Joule heating, numerical simulations were performed with a theoretical model containing a fully coupled electrostatic, energy, and Stokes system. The accuracy of this numerical model was previously validated with the thermoreflectance method for a similar system. According to these simulations, the optimum AC application conditions were 6 Vpp (pp: peak to peak) and 100 kHz. Under these conditions, the ACET at the gate electrode is predicted to cause an average temperature rise of only a few degrees Celsius in the buffer while the average velocity of the water can reach 15 μm s⁻¹ (data not shown). This speed can be sufficient to rapidly transport the target proteins to the sensor surface. For the experimental verification of the simulations, the magnitude of the voltage applied to the gate electrode was varied and the electrochemical impedance spectra of the electrode was recorded right after the stimulation. Increasing the amplitude from 2 VPP to 6 VPP had a relatively small impact on the spectra (i.e., the diameter of the semicircle decreased with an increase in solution temperature) (data not shown). At a critical voltage of 8 Vpp and above, however, the impedance decreased drastically, showing that the biochemical layer was removed from the gold electrode surface. To determine how long the stimulation should be applied for, sensing measurements were performed, using various incubation times, that is, 30, 60, and 120 s (data not shown). The results showed that the sensor response is maximized if the gate electrode is incubated with the target sample for 2 min under ACET (data not shown).

The ACET, applied using these conditions, were evaluated to determine whether it allows for the specific binding events to occur on the gate electrode.

A GFP nanobody construct that is specific to GFP was immobilized on the gate electrode for the detection of GFP since this binding event can also be monitored with a fluorescence microscope (data not shown). After 2 min of ACET-assisted incubation of the gate electrode with a GFP solution (1×10⁻⁹ m), fluorescent signals were detected on the electrode. Conversely, under the same conditions, the GFP-nanobody electrodes did not show a fluorescence response to mCherry protein, which is a red fluorescent protein with low sequence homology but very high structural similarity to GFP. Similar measurements were performed using diffusion-aided incubation, that is, allowing the protein solution to remain on the electrode surface for 10 min with some intermittent manual mixing. In contrast to the ACET-assisted experiments, some nonspecific binding of mCherry on the electrode surface were observed. It is speculated that the ACET, by creating a continuous flow parallel to the electrode surface, may help wash away nonspecific or low-affinity antigens that were only partially released in the traditional washing step. It is thus expected to have a lower background signal from the sensors with ACET-assisted incubation compared to those with the diffusion-based one. Having determined ACET application parameters and verified its effect on reducing the binding of unspecific analytes, the Ty1- and GFP-nanobody-functionalized gate electrodes were included with 10 μL of the buffer solutions containing increasing concentrations of the target analyte (SARS-CoV-2 spike protein, S1) for 2 min while applying the AC simulation (FIG. 8C). GFP-nanobody electrodes were used as negative control as the GFP nanobody should not bind to spike protein although some non-specific binding were observed in subsequent QCM-D experiments (data not shown).

Comparison of p-Type and n-Type OECT Performance in Protein Detection

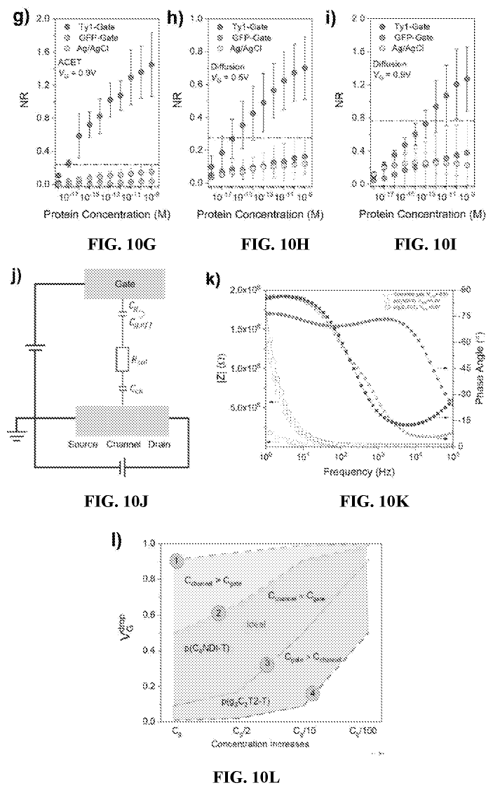

After the ACET-assisted sample incubation step, the gate electrodes were integrated with the two types of OECT channels and recorded device current-voltage characteristics. In FIGS. 10A-10D, the transfer curves were plotted (ID vs VG), which were recorded every time the gate was exposed to a new solution. Binding of SARS-CoV-2 S1 on the gate electrode modulated the current flowing in both channels. For the p(g₃C₂T2-T) OECT, protein binding increased the ID at all gate voltages applied (FIG. 10A) accompanied with a shift of the transfer curve toward more positive VG values (data not shown). For the p(C6NDI-T) device, the target binding caused a similar shift, however, because of the bell-shaped profile of the transfer curve, ID underwent a marked reduction at low VG regime (FIG. 10C). At high VG, the ID value increased as a function of SARS-CoV-2 S1 concentration. The shift of the transfer curves with analyte binding was attributed to the positively charged nature of the protein at pH 7.4. The bound proteins induced an electric dipole at the gate/electrolyte interface and push the threshold voltage to more positive values. Conversely, both sensors produced only a weak response when the GFP-nanobody gate electrodes were incubated with the same protein solutions (FIGS. 10B and 10D). To compare the sensing performance of these devices, the normalized change in the drain currents (NR=|ID−I0|/I0) at chosen biasing conditions was plotted as a function of protein concentrations. The reference (blank) response of the nanobody-OECT sensors (I0) was recorded before exposing them to analyte-containing solutions. The VD at the linear regime was chosen and VG values that maximized the binding induced changes in the current. The current changes were maximized near the Vth for the p(g₃C₂T2-T), and at two voltages for p(C₆NDI-T); at VG=0.5 V, close to the Vth, and at VG=0.9 V. FIGS. 10E-10G show these calibration curves (normalized response (NR) vs protein content). These calibration curves also include the channel current measured with an Ag/AgCl gate electrode. After each protein sensing measurement, an Ag/AgCl gate was used to account for any contributions to the sensor response related to channel material instability. These control measurements were necessary considering the S-shaped/D vs VD curves at high gate voltages (data not shown). As an additional investigation of stability, EIS, electrochemical QCM-D, and in situ Fourier-transform infrared spectroscopy measurements were performed (data not shown) for the n-type film. The results showed that high doping voltages do not cause material degradation (data not shown). At both gate biasing conditions, the p(C₆NDI-T) current change in response to 100×10⁻¹⁸ m of spike protein was well distinguishable from the response of the GFP gate electrodes (FIGS. 10F and 10G). The response of the p(g₃C₂T2-T) sensor to the target became significantly different from the GFP electrode gated device only at and above 100×10⁻¹⁵ m (FIG. 10E). Comparing these plots, it is concluded that the NR values of the n-type OECT are higher than the p-type device, especially when reported at VG=0.9 V. Although the p-type OECT outperforms the n-type in terms of transistor gain (gm 60 mS vs gm 15 μS), when it was operated using the same biofunctionalized gates, the n-type OECT lowered the limit of detection and shows higher current response to a change in the protein concentrations. The high performance of the p(C6NDI-T) OECT is complemented by a low power demand (100 nW) compared to the p-type (100 μW), which makes the p(C₆NDI-T) OECT compatible with integrated circuit designs and a handheld battery-driven reader.

The binding between the nanobody and the SARS-CoV-2 S1 causes an impedance increase at the gate electrode. The capacitance of the protein-bound gate electrode decreases (C_{g,eff}<C_{g}), leading to an increase in the voltage drop at the gate electrode/electrolyte interface (V_{G}^{drop}) and a change in the capacitive coupling between the gate and channel (FIG. 10J). The differences in the sensor behavior between the two OECTs can be understood when comparing the capacitance ratios of the gate and channel (FIG. 3K). For OECTs to have high gain, the aim is to have Cg as large as possible compared to the channel capacitance (e.g., C_{g}=100C_{ch}, device 4 in FIG. 10L). However, for devices where the gate/channel capacitance ratio is too large, small changes in C_{g} do not have an impact on the already very large voltage drop across the channel (V_{CH}^{drop}). This is the situation for the p(g₃C₂T2-T) channel, which has a very small capacitance (33±5 nF) compared to that of the gate electrode (1.23 μF). Owing to its higher channel capacitance (146±11 nF) and, consequently, smaller C_{g}/C_{ch}, the p(C6NDI-T)-based OECT generates a noticeable change in V_{CH}^{drop} and ID even for a few binding events at the gate. Devices with lower gate and channel capacitance differences make for more sensitive sensors. If the channel has however higher capacitance than the gate (e.g., Cg=0.1C_{ch}, device 1 in FIG. 31), the OECT is not an effective amplifying transducer. FIG. 10L illustrates that with a change of channel materials and/or channel/gate geometries, it is possible to design immunosensors with particular sensitivity toward low or high concentration of analytes and to adjust their dynamic range. It is noted that capacitance decrease is not the only parameter governing the sensor operation mechanism as the bound proteins on the gate electrode also change the electrochemical potential of that terminal.

The Effect of ACET on the n-Type OECT Sensor Performance

The ACET-facilitated sample incubation (2 min) approach was compared with the conventional method described previously (manual up and down pipetting for 30 s every 3 min during a 10 min incubation) (FIG. 10F vs. 10H and FIG. 10G vs. 10I). The SARS-CoV-2 S1 detection performance of the ACET-driven and the conventional diffusion-based platform was similar, reaching a detection limit as low as 1×10⁻¹⁵ m at VG=0.5 V (FIGS. 10F and 10H). As discussed above, the dense nanobody layer on the electrode surface creates a very high local receptor concentration (>100×10⁻⁶ m) such that binding is no longer dictated by the dissociation constant of the receptor-target interaction (25×10⁻⁹ m). Instead, binding becomes diffusion limited. Improved mixing can directly affect this diffusion-limited transport of target to the gate surface. Compared to the conventional incubation method, ACET not only had the advantage of a much-reduced incubation time, but also gave higher NR values, especially for low protein concentrations. Apart from the diffusion limited transport of target to the gate surface, manual incubation may also lead to the loss of target from the surface by adsorption to the pipette tip during repeated mixing. Further, the ACET flow parallel to the electrode surface may improve the washing away of nonspecific or low-affinity antigens, resulting in lower background signal compared to the diffusion-based sensing. Overall, ACET accelerated the transport of target molecules to the electrodes, reduced incubation times from ten down to 2 min, and minimized manual intervention during the sample incubation step.

### ACET-Assisted Protein Sensing in Complex Biological Media

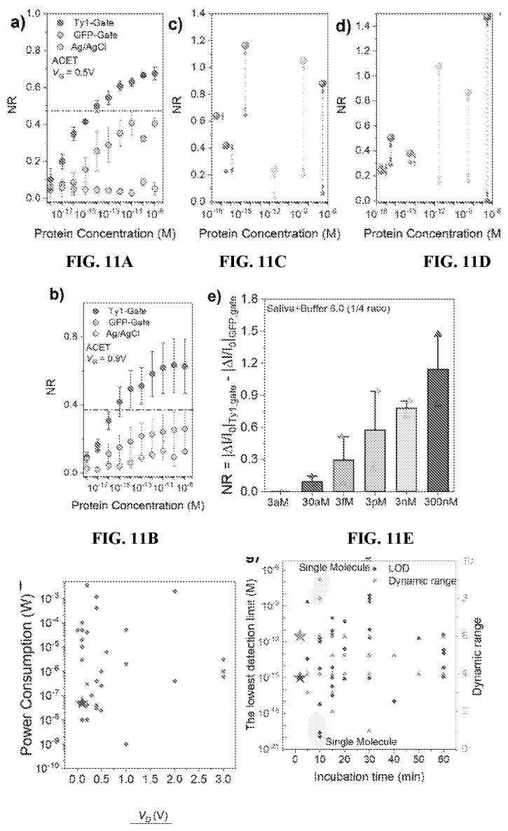

A recent work compared saliva and nasopharyngeal samples from the same patients and concluded that saliva samples reduced variability and increased titer detection and the consistency with RT-PCR. The self-collection of saliva is easy, non-invasive, and minimizes the interaction between patients and healthcare personnel, making saliva a preferred medium for diagnostic applications. Therefore, the ACET integrated nanobody-OECT sensors was challenged with human saliva as the sample medium. Three Ty1-functionalized gate electrodes were sequentially exposed to increasing concentrations of SARS-CoV-2 S1 protein spiked into a 1:3 (v/v) saliva: lysis buffer mixture. As before, the NR increased with increasing target concentration (FIGS. 11A and 11B). The resulting calibration curve (FIG. 11A) showed that the Ty-1-nanobody-OECTs detected spike protein concentrations as low as 10×10⁻¹⁵ M. As a negative control, three GFP-nanobody-functionalized gate electrodes were exposed to the same spike protein/saliva samples. These control gate electrodes showed a (albeit lower) response to the increasing spike protein concentrations.

The repeated exposure to the complex saliva: buffer: target mixture may eventually lead to the accumulation of unspecific background binding to any component of the biolayer or HDT surface. By contrast, in a clinical application, each gate will only meet a single patient sample. This clinical scenario was recapitulated by exposing individual gate electrodes to randomly selected saliva samples with different SARS-CoV-2 S1 concentrations (large circles in FIGS. 11C and 11D). Again, the same number of GFP-nanobody-functionalized gate electrodes were used as controls to validate the specificity (small circles in FIGS. 11C and 11D). These individual gate electrode measurements in FIGS. 11C and 11D showed that the Ty-1-nanobody OECT always gave higher NR values than the GFP-nanobody gated channels (compare the small circles vs the large ones in each plot) but the NR values varied widely and seemed not to correlate with target concentration. Protein sensing in saliva, that is, an unprocessed (no heating, filtering, or centrifugation steps) sample with a complex content that varies from patient to patient, thus, has proven to be challenging. It was hypothesized that absolute NR values may have also been affected by variation in the Au electrode surface or biofunctionalization. Therefore, different gates, all prepared in parallel under identical conditions, were subjected to EIS and XPS measurements before and after target binding and indeed observed considerable variability in capacitance (data not shown). Further modification of fabrication and biofunctionalization methods may help to address this gate-to-gate variation as well as background binding. The larger size of these gate electrodes may be a factor increasing the standard deviation of sensor output. The larger geometry reduces the surface homogeneity and makes it harder to fabricate gate electrodes with the same quality of immobilized chemical and biological layers. However, the comparison of several target and negative control measurements with multiple gates per sample can efficiently suppress both of these confounding factors. The difference between the average of the target and negative control measurements indeed correlated with protein concentration over a wide dynamic range (FIG. 11E). These results thus demonstrated the sensitivity and selectivity of ACET-assisted nanobody OECTs in complex biological media and at physiologically and clinically relevant protein concentrations.

Finally, the nanobody/OECT sensor performance was compared to other similar thin film transistor and electrode-based devices developed for immunosensing applications and benchmark the performance of this technology in terms of power consumption, LOD, incubation time, and dynamic range (FIGS. 11F and 11G and Table 3). Although there are several biosensors showing merits including single molecule detection, nW power requirement, and 1 min of incubation time, the device described herein can be operated using only 100 nW power to operate and 2 min of incubation with a 10 μL sample to detect target proteins as low as 30×10⁻¹⁸ M up to 300×10⁻⁹ M concentrations in complex media.

TABLE 3
Performance parameters of selected electrochemical
immunosensors for various protein antigens

| | | | Dynamic | | | | | |
|---|---|---|---|---|---|---|---|---|
| Ref.* | LOD | | Range | | Incubation | | Media | Target |
| This | 100 | aM | 30 aM- | | 2 | min | PBS/Saliva | SARS-CoV-2 S1 |
| Work | | | 300 nM | | | | | |
| [8] | 2.2 | nM | 0.5-5000 | ng/mL | 5 | min | PBS | trinitrotoluene |
| | | | | | | | | (TNT), |
| [9] | 0.1 | nM | 0.01-.100 | nM | — | | PBS | heat-shock |
| | | | | | | | | protein |
| [10] | 1.1 | fM | 100 fg/mL- | | — | | Serum | Prostate specific |
| | | | 100 ng/mL | | | | and | antigen/a1- |
| | | | | | | | PBS | antichymotrypsin |
| | | | | | | | | (PSA-ACT) |
| [11] | 4 | pM | 10⁻⁴-10² | μg/mL | 1 | h | Serum | C-reactive |
| | | | | | | | and | protein (CRP) |
| | | | | | | | PBS | |
| [12] | 0.94 | fM | 22.4 fg/mL- | | | | PBS | detection of |
| | | | 22.5 ng/mL | | | | | hepatitis B |
| | | | | | | | | surface antigen. |
| [13] | 1 μg/mL- | | | | 30 | min | artificial | human |
| | 50 μg/mL | | | | | | saliva | chromogranin A |
| [14] | 10 | aM | 10 aM- | | 40 | MIN | PBS | H5N2 avian |
| | | | 1 pM | | | | | influenza virus |
| [15] | 1 | fM | | | real | | PBS | Hepatitis B |
| | | | | | time | | | marker (HBsAg) |
| | | | | | | | | and cancer |
| | | | | | | | | marker - |
| | | | | | | | | fetoprotein |
| | | | | | | | | (AFP) |
| [16] | 0.11 | pM | 0.02-30 | mIU/L | 1 | h | PBS | Human thyroid |
| | | | | | | | | stimulating |
| | | | | | | | | hormone |
| [17] | 1.2 | fM | 1 fg/mL | | | | PBS | oral squamous |
| | | | 1 ng/mL 1 | | | | | cell carcinoma |
| | | | | | | | | Interleukin-8 |
| | | | | | | | | (IL- 8) and |
| | | | | | | | | tumor necrosis |
| | | | | | | | | factor |
| [18] | 33.33 | aM | 1 fg/mL | | real | | PBS | Cancer |
| | | | 1 ng/Ml | | time | | | biomarkers |
| | | | | | | | | cytokeratin 19 |
| | | | | | | | | fragment |
| | | | | | | | | (CYFRA21-1) |
| | | | | | | | | and prostate |
| | | | | | | | | specific antigen |
| [19] | 0.2 | fM | 5 fg/mL- | | real | | PBS | Prostate cancer |
| | | | 500 pg/mL | | time | | | biomarker PSA |
| [20] | 7.1 | fM | 500 fg/mL- | | real | | Serum | AFP and |
| | | | 50 ng/mL | | time | | and | carcinoembryonic |
| | | | for AFP | | | | PBS | antigen (CEA) |
| | | | 50 fg/mL- | | | | | primary hepatic |
| | | | 10 ng/mL | | | | | |
| | | | for CEA | | | | | |
| [21] | 3.85 | pM | 0.092 ng/mL- | | Real | | PBS | Cardiac troponin |
| | | | 46 ng/mL | | time | | | I (cTnI) |
| | | | | | | | | biomarker for |
| | | | | | | | | acute myocardial |
| | | | | | | | | infarction |
| [22] | 0.21 | pM | 5 pg//mL- | | | | PBS | Cardiac disease |
| | | | 1 5 ng/mL | | | | | biomarker cTnI |
| 11 | 3.98 | pM | 10−4 to | | | | PBS | C-reactive |
| | | | 102 μg/mL | | | | | protein (CRP) |
| | | | | | | | | inflammatory |
| | | | | | | | | biomarker |
| | | | | | | | | related with |
| 23 | 6.82 | fM | 1 pg/mL- | | 60 | MIN | PBS | Prostate cancer |
| | | | 1 μg/mL | | | | and | biomarker |
| | | | | | | | human | osteopontin |
| | | | | | | | serum | (OPN) |
| 24 | 2.76 | pM | 1 pg/mL- | | 10 | min | PBS | Stress biomarker |
| | | | 1 μg/mL | | | | | cortisol in saliva |
| 25 | 50 | pM | 1 ng/mL- | | | | PBS | Lyme disease |
| | | | 3000 ng/mL | | | | | antigen |
| 26 | 1 | pM | 1 pM- | | real | | PBS | Human |
| | | | 10 nM | | time | | | immunodeficiency |
| | | | | | | | | virus (HIV) |
| 27 | 8 | nM | 1 μg/mL- | | 30 | min | PBS/ | glycoproteins |
| | | | 50 μg/mL | | | | artificial | (immunoglobulin |
| | | | | | | | saliva | A, |
| | | | | | | | | immunoglobulin |
| | | | | | | | | G, and |
| | | | | | | | | chromogranin A |
| [28] | 72 | fM | 2.4 × 10−12 g/mL- | | 30 | min | PBS - | inactivated ebola |
| | | | 1.2 × 10−7 g/mL | | | | Serum | virus (EBOV). |
| [29] | 4.1 | nM | up to | | 30 | min | PBS | IgG |
| | | | 10 μg/mL | | | | | |
| [30] | 0.8 | fM | 0.8 fM- | | 20 | min | whole | thyroid hormone |
| | | | 1 nM | | | | serum | |
| [31] | 0.25 | pM | 0.25 pM- | | | | PBS | alfa synuclein |
| | | | 25 nM | | | | | |
| [32] | 2.2 | pM | 0.8 | pm- | | | PBS | procalcitonin |
| [33] | 2 | pM | 5 | pM- | 50 | min | PBS | microRNA21 |
| [34] | — | | 0.025-1 | HAU | real | | PBS | human influenza |
| | | | | | time | | | A virus |
| [35] | 10.5 | pM | | | 30 | minutes | PBS | interleukin-6 |
| | | | | | | | | (IL-6), |
| [36] | 2 | pM | 4 pM- | | 10 | min | PBS | C-reactive |
| | | | 2 μM | | | | | protein (CRP) |
| [13] | 2.2 | nM | 11-50 | μg/mL | 30 | min | PBS | human |
| | | | | | | | | chromogranin A |
| [37] | 8.2 | pM | 5 ng/mL- | | | | PBS | Protein G |
| | | | 50 μg/mL | | | | | |
| [38] | 38 | fM | 100 pg/mL- | | 5 | min | PBS | prostate specific |
| | | | 100 ng/mL | | | | | antigen/1- |
| | | | | | | | | antichymotrypsin |
| | | | | | | | | (PSA-ACT) |
| [39] | 6 | fM | 6 fM- | | 10 | min | PBS | Anti-IgG |
| | | | 60 pM | | | | | |
| [40] | 6 | aM | 6 aM- | | 10 | min | PBS | IgG |
| | | | 6 pM | | | | | |
| [41] | 25 | zM | 0.6 zM- | | 10 | min | PBS | IgG |
| | | | 6 × 10⁷ zM | | | | | |
| [42] | 11 | zM | 1 zM-1 pM | | 10 | min | PBS | IgG |
| [43] | 150 | ng/mL | 0.16 μg/mL- | | — | | saliva:PBS | spike protein |
| | | | 1.25 | | | | | |
| [44] | 1 | fg/mL | 1 fg/mL- | | 10 | min | PBS | RNA (fragment |
| | | | 1 pg/mL | | | | | of the RdRp |
| | | | | | | | | gene sequence in |
| | | | | | | | | SARS- CoV-2) |
| [45] | 26 | fM | 0.1-1000 | pM | 3 | h | Tris- | a 26-nt-long |
| | | | | | | | HCl | ORF1ab |
| | | | | | | | buffer | fragment of |
| | | | | | | | | SARS-CoV-2 |
| | | | | | | | | RNA |
| [46] | 1.68 | ng/mL | 1-100 | ng/mL | 30 | min | Saliva + | spike protein |
| | | | | | | | virus | |
| | | | | | | | inactivation | |
| | | | | | | | solution | |
| | | | | | | | (Triton X) | |
| | | | | | | | (VPSS | |
| | | | | | | | solution) | |
| [47] | 4000 | | 4 × 103−4 × | | 10 | min | Buffer | spike protein |
| | pseudovirus | | 107 virus | | | | Saliva | |
| | buffer | | particle/mL | | | | | |
| [48] | 1 | ag/mL | 0.1 fg/mL- | | 1 | h | 10% | spike protein |
| | | | 1000 fg/mL | | | | saliva | |
| [49] | 4 | gc/μL | | | | | Viral | spike protein |

| [50] | 111 | f | 50 fM- | 15 | min | Transport | spike protein |
|---|---|---|---|---|---|---|---|
| | | | 350 fM | | | medium | |
| | | | | | | Lysis | |
| | | | | | | buffer | |
| [50] | 0.1 | ug/ml | 1 nM- | 21 | min | PBS | spike protein |
| | | | 100 mM | | | | |
| [51] | 1 | fg/mL | 1 fg/mL- | 3 | min | PBS | spike protein |
| | | | 2.5 μg/mL | | | | |
| | | | 10 pg/mL | | | | |
| [52] | 1.7 | fM | 10 pg/mL- | t > 1 | h | PBS | nucleocapsid |
| | | | 200 pg/mL | | | | gene |
| [53] | 1 | fg/mL | 1 fg/mL-1 | 15 | min | Saliva | spike protein |
| [54] | 33 | fg/mL | 0.1 pg/mL- | 30 | min | PBS | synthetic DNA |
| | | | 1 mg/mL | | | | |
| [55] | 10 copies | | — | 20 | min | Buffer | proton numbers |
| | per reaction | | | | | | during the DNA |
| | | | | | | | amplification |
| [56] | 2 | pg/mL | 100 fg/mL- | 4 | min | Saliva | spike protein |
| | | | 100 ng/mL | | | | |
| [57] | 230 | pg/mL | 0.01 ng/mL- | 1 | h | Whole | nucleocapsid |
| | | | 1 ng/mL | | | serum | |
| [58] | 0.15 | ng/mL | 0.1 ng/mL- | t > 45 | min | PBS | spike protein |
| | | | 100 ng/mL | | | | |
| [59] | 1 | fg/mL | 1 fg/mL- | 0.05 | s | Artificial | spike protein |
| | | | 10 pg/mL | | | saliva | |
| [60] | 10 | pM | 10 pM- | 5 | min | 50% | spike protein |
| | | | 1 nM | | | artificial | |
| | | | | | | saliva | |
| [61] | 2.29 | fM | 1 fM- | 2 | min | Throat | SARS-CoV-2 |
| | | | 100 pM | | | sample | RdRp gene |
| [62] | 100 | fg/mL | 1 fg/mL- | 2.5 | min | Viral | spike protein |
| | | | 10 pg/mL | | | Transport | |
| | | | | | | Media | |
| [63] | 0.1 | mg/mL | 0.1 mg/mL- | 3 | min | Serum | anti-SARS-CoV- |
| | | | 10 mg/mL | | | (1/100) | 2 monoclonal |
| | | | | | | dilution | antibody |
| | | | | | | factor | |
| [64] | 0.55 | fg/mL | 0.55 fg/mL- | 2 | min | PBS | anti-SARS-CoV- |
| | | | 55 | | | | 2 spike protein |
| | | | | | | | antibody |
| [65] | 1 | copy/μL | μg/mL | 2 | hours | nasal | capture probe |
| | | | 1 to 10⁹ | | | swab | |
| | | | copies/μL | | | | |
| [66] | 6.9 | copy/μL | 585-5854 × | 5 | min | Viral | ssDNA |
| | | | 10⁷ copies/μL | | | Transport | |
| | | | | | | Media | |
| [67] | 0.5 | ng/mL | 5 ng/mL- | 10 | min | Saliva | antibody |
| | | | 500 ng/mL | | | | |
| [68] | 25 | fg/μL | 25 fg/μL- | 1 | min | 0.01x | spike protein |
| | | | 10 1 minng/ | | | PBS | |
| | | | μL | | | | |
| [69] | 10 | nM | — | 1 | h | diluted | SARS-CoV-2 |
| | | | | | | PBS | RdRp gene |

| | | | | | |
|---|---|---|---|---|---|
| [69] | 1.68 × 1022 | — | — | PBS | spike protein |
| | μg/mL | | | | |

| | | | | | | | Power | |
|---|---|---|---|---|---|---|---|---|
| Ref.* | Sensing Method | Configuration | I_{D} | | V_{D} | | Consumption | |
| This | Amperometric | OECT | 100 | nA | 0.1 | V | 0.1 | μW |
| Work | | | | | | | | |
| [8] | Amperometric | Chemiresistive/ | 0.0025-0.0175 | μA | 0.1-0.5 | V | 1 | μW |
| | | field-effect | | | | | | |
| | | transistor | | | | | | |
| [9] | Amperometric | graphene | 0.05-0.1 | μA | 0.1 | V | 10 | μW |
| | | field-effect | | | | | | |
| | | transistors | | | | | | |
| [10] | Amperometric | Graphene oxide | | | | | | |
| | | field-effect | | | | | | |
| | | transistor | | | | | | |
| [11] | Amperometric | single-walled | 0-200 | nA | 0-2 | V | 0.4 | μW |
| | | carbon | | | | | | |
| | | nanotubes field | | | | | | |
| | | effect transistor | | | | | | |
| [12] | Amperometric | Ion-sensitive | 0-300 | μA | 0-3 | V | 1 | μW |
| | | field-effect | | | | | | |
| | | transistors | | | | | | |
| [13] | Amperometric | extended-gate | 0-0.3 | μA | 0.3 | V | 0.1 | μW |
| | | organic | | | | | | |
| | | fieldeffect | | | | | | |
| | | transistor | | | | | | |
| [14] | Amperometric | silicon nanowire | 0-1500 | nA | 0.03 V | | 0.05 | μW |
| | | SiNW field | | | (1 V) | | | |
| | | effect transistor | | | | | | |
| [15] | Amperometric | silicon nanowire | 5 × 10−7- | | 0.2 | V | 0.3 | μW |
| | | SiNW field | 1.5 × 10−6A | | | | | |
| | | effect transistor | | | | | | |
| [16] | Amperometric | silicon nanowire | 30-50 | μA | 1 | V | 50 | μW |
| | | SiNW field | | | | | | |
| | | effect transistor | | | | | | |
| [17] | Amperometric | silicon nanowire | 60 | nA | 0.4 | V | 0.03 | μW |
| | | SiNW field | | | | | | |
| | | effect transistor | | | | | | |
| [18] | Amperometric | silicon | 2 | μA | 1 | V | 2 | μW |
| | | nanowire SiNW | | | | | | |
| | | field effect | | | | | | |
| | | transistor | | | | | | |
| [19] | Amperometric | silicon nanowire | 1 | nA | 1 | V | 1 | nW |
| | | SiNW field | | | | | | |
| | | effect transistor | | | | | | |
| [20] | Amperometric | silicon nanowire | 50 | nA | 0.5 | V | 25 | nW |
| | | SiNW field | | | | | | |
| | | effect transistor | | | | | | |
| [21] | Amperometric | silicon nanowire | 0.1 | μA | 0.2 | V | 10 | nW |
| | | SiNW field | | | | | | |
| | | effect transistor | | | | | | |
| [22] | Amperometric | silicon nanowire | 1 | nA | 1 | V | 1 | nW |
| | | SiNW field | | | | | | |
| | | effect transistor | | | | | | |
| 11 | Amperometric | silicon nanowire | | | | | | |
| | | SiNW field | | | | | | |
| | | effect transistor | | | | | | |
| 23 | Amperometric | silicon nanowire | 1 | mA | 2 | V | 2 | mW |
| | | SiNW field | | | | | | |
| | | effect transistor | | | | | | |
| 24 | Amperometric | silicon nanowire | 10 | μA | 0.6-0.6 | V | 6 | μW |
| | | SiNW field | | | | | | |
| | | effect transistor | | | | | | |
| 25 | Amperometric | silicon nanowire | 10 | nA | — | | 10 | nW |
| | | SiNW field | | | | | | |
| | | effect transistor | | | | | | |
| 26 | Amperometric | graphene field- | 5 | mA | 0.01 | V | 50 | μW |
| | | effect transistors | | | | | | |
| 27 | Amperometric | Polymer field- | 1 | μA | 3 | V | 3 | μW |
| | | effect transistors | | | | | | |
| [28] | Amperometric | field effect | 30 | μA | 0.1 | V | 3 | μW |
| [29] | Amperometric | OFET | 1 | μA | 3 | V | 3 | μW |
| [30] | Amperometric | graphene field- | 200 | μA | 0.1 | V | 20 | μW |
| | | effect transistors | | | | | | |
| [31] | Amperometric | EGOFET | 0.1 | μA | 0.1 | V | 10 | nW |
| [32] | Amperometric | EGOFET | 2 | μA | 0.5 | V | 1 | μW |
| [33] | Amperometric | OECT | 0.2 | mA | 0.2 | V | 40 | μW |
| [34] | Amperometric | OECT | 0.5 | mA | 0.1 | V | 50 | μW |
| [35] | Amperometric | OECT | 1 | mA | 0.1 | V | 100 | μW |
| [36] | Amperometric | OECT | 2 | μA | 0.5 | V | 1 | μW |
| [13] | Amperometric | OFET | 0.2 | μA | 3 | V | 0.6 | μW |
| [37] | Amperometric | EGOFET | 0.2 | μA | 0.2 | V | 40 | nW |
| [38] | Amperometric | OECT | 7 | mA | 0.2 | V | 3.5 | mW |
| [39] | Amperometric | OECT | 1 | mA | 0.4 | V | 0.4 | mW |
| [40] | Amperometric | OECT | 3 | mA | 0.4 | V | 1.2 | mW |
| [41] | Amperometric | OFET | 0.1 | μA | 0.4 | V | 40 | nW |
| [42] | Amperometric | OFET | 0.2 | μA | 0.4 | V | 40 | nW |

| | | | | | |
|---|---|---|---|---|---|
| [43] | Impedimetric | Electrode | — | — | — |

| | | | | | | | | |
|---|---|---|---|---|---|---|---|---|
| [44] | Amperometric | OFET | 50 | μA | 0.05 | V | 2.5 | μW |

| | | | | | |
|---|---|---|---|---|---|
| [45] | Voltametric | Electrode | — | — | — |
| [46] | Impedimetric | Electrode | — | — | — |
| [47] | Amperometric | Electrode | — | — | — |
| [48] | Amperometric | Electrode | — | — | — |

| | | |
|---|---|---|
| [49] | Voltametric | Electrode |
| [50] | Amperometric | Electrode |
| [50] | Impedimetric | Electrode |
| [51] | Impedimetric | Electrode |
| [52] | Voltametric | Electrode |
| [53] | Amperometric | MOSFET |
| [54] | Voltametric | Electrode |
| [55] | Amperometric | Electrode |
| [56] | Impedimetric | Electrode |
| [57] | Amperometric | Electrode |
| [58] | Amperometric | Electrode |
| [59] | Amperometric | OFET |
| [60] | Amperometric | Electrode |
| [61] | Amperometric | OFET |
| [62] | Amperometric | OFET |
| [63] | Impedimetric | Electrode |
| [64] | Amperometric | OFET |
| [65] | Voltametric | Electrode |
| [66] | Voltametric | Electrode |
| [67] | Voltametric | Electrode |
| [68] | Amperometric | Electrode |
| [69] | Impedimetric | Electrode |
| [69] | Voltametric | Electrode |

### *LIST OF REFERENCES

### Conclusion

Described herein is a label-free electrochemical immunosensing technology with ultrarapid detection ability, 100×10⁻¹⁸ m detection limit in buffer and diluted saliva, and a large dynamic range (from ×10⁻¹⁸ m to ×10⁻⁹ m). A new solution-processable n-type organic semiconductor allowed the transistor to operate in enhancement mode, with high sensitivity and selectivity at very low biasing conditions. ACET-induced mixing significantly reduced the time required for immunocomplex formation at the nanobody-functionalized gate electrode as it affected the diffusion-limited transport of proteins to the gate electrode surface. ACET also enhanced the detection sensitivity with a lower standard deviation of the sensor response compared to the operation with diffusion-controlled incubation. The ACET enhanced nanobody-OECT biosensor detected specific protein molecules from unprocessed saliva samples in ambient conditions after only 2 min of incubation. Measurements in complex samples used averaged measurements and off-target controls. This platform can be adapted to detect any other targets with a change of the biorecognition unit, and its reusability, simple operation, and speed of detection can be used for routine biomarker screening.

### Example 3. OECT Functionalization with CA9/sgRNA

### Materials and Methods

### Construct Design

Cas proteins were fused to SpyCatcher a 3C protease cleavage site and a 8× Histidine purification tag on a N (dSauCas9) or C (all Cas13 proteins) terminal based on the available structures in the Protein Data Bank (PDB) (dSauCas9: 5AXW, dLwaCas13a: Not available, dLbuCas13a: 5XWP and dEsCas13d: 6E9F). Protein sequences were codon-optimized for expression in E. coli and commercially gene synthesized (Twist) in the high copy cloning vector pJEx411c (a modified version of the original DNA 2.0/ATUM) that contains Kanamycin resistance and RBS insulator (BCD2) cassette to improve translation initiation⁶⁸. Protein sequences are shown in Table 4.

TABLE 4
Protein sequences

| Protein Name | Protein Sequence |
|---|---|
| His8-3C-SpyC- | MTGHHHHHHHHSLEVLFQGPTGSVDTLSGLSSEQGQSGDMTI |
| dSauCas9 | EEDSATHIKFSKRDEDGKELAGATMELRDSSGKTISTWISDGQ |
| | VKDFYLYPGKYTFVETAAPDGYEVATAITFTVNEQGQVTVN |
| | GKATKGDAHIGSGTGMGKRNYILGLAIGITSVGYGIIDYETRD |
| | VIDAGVRLFKEANVENNEGRRSKRGARRLKRRRRHRIQRVK |
| | KLLFDYNLLTDHSELSGINPYEARVKGLSQKLSEEEFSAALLH |
| | LAKRRGVHNVNEVEEDTGNELSTKEQISRNSKALEEKYVAEL |
| | QLERLKKDGEVRGSINRFKTSDYVKEAKQLLKVQKAYHQLD |
| | QSFIDTYIDLLETRRTYYEGPGEGSPFGWKDIKEWYEMLMGH |
| | CTYFPEELRSVKYAYNADLYNALNDLNNLVITRDENEKLEYY |
| | EKFQIIENVFKQKKKPTLKQIAKEILVNEEDIKGYRVTSTGKPE |
| | FTNLKVYHDIKDITARKEIIENAELLDQIAKILTIYQSSEDIQEE |
| | LTNLNSELTQEEIEQISNLKGYTGTHNLSLKAINLILDELWHTN |
| | DNQIAIFNRLKLVPKKVDLSQQKEIPTTLVDDFILSPVVKRSFI |
| | QSIKVINAIIKKYGLPNDIIIELAREKNSKDAQKMINEMQKRNR |
| | QTNERIEEIIRTTGKENAKYLIEKIKLHDMQEGKCLYSLEAIPL |
| | EDLLNNPFNYEVDHIIPRSVSFDNSFNNKVLVKQEEASKKGNR |
| | TPFQYLSSSDSKISYETFKKHILNLAKGKGRISKTKKEYLLEER |
| | DINRFSVQKDFINRNLVDTRYATRGLMNLLRSYFRVNNLDVK |
| | VKSINGGFTSFLRRKWKFKKERNKGYKHHAEDALIIANADFIF |
| | KEWKKLDKAKKVMENQMFEEKQAESMPEIETEQEYKEIFITP |
| | HQIKHIKDFKDYKYSHRVDKKPNRELINDTLYSTRKDDKGNT |
| | LIVNNLNGLYDKDNDKLKKLINKSPEKLLMYHHDPQTYQKL |
| | KLIMEQYGDEKNPLYKYYEETGNYLTKYSKKDNGPVIKKIKY |
| | YGNKLNAHLDITDDYPNSRNKVVKLSLKPYRFDVYLDNGVY |
| | KFVTVKNLDVIKKENYYEVNSKCYEEAKKLKKISNQAEFIAS |
| | FYNNDLIKINGELYRVIGVNNDLLNRIEVNMIDITYREYLENM |
| | NDKRPPRIIKTIASKTQSIKKYSTDILGNLYEVKSKKHPQIIKKG |
| | (SEQ ID NO: 30) |
| dLwaCas13a- | MTGMKVTKVDGISHKKYIEEGKLVKSTSEENRTSERLSELLSI |
| SpyC-3C-His8 | RLDIYIKNPDNASEEENRIRRENLKKFFSNKVLHLKDSVLYLK |
| | NRKEKNAVQDKNYSEEDISEYDLKNKNSFSVLKKILLNEDVN |
| | SEELEIFRKDVEAKLNKINSLKYSFEENKANYQKINENNVEKV |
| | GGKSKRNIIYDYYRESAKRNDYINNVQEAFDKLYKKEDIEKL |
| | FFLIENSKKHEKYKIREYYHKIIGRKNDKENFAKIIYEEIQNVN |
| | NIKELIEKIPDMSELKKSQVFYKYYLDKEELNDKNIKYAFCHF |
| | VEIEMSQLLKNYVYKRLSNISNDKIKRIFEYQNLKKLIENKLL |
| | NKLDTYVRNCGKYNYYLQVGEIATSDFIARNRQNEAFLRNII |
| | GVSSVAYFSLRNILETENENGITGRMRGKTVKNNKGEEKYVS |
| | GEVDKIYNENKQNEVKENLKMFYSYDFNMDNKNEIEDFFANI |
| | DEAISSIAHGIVHFNLELEGKDIFAFKNIAPSEISKKMFQNEINE |
| | KKLKLKIFKQLNSANVFNYYEKDVIIKYLKNTKFNFVNKNIPF |
| | VPSFTKLYNKIEDLRNTLKFFWSVPKDKEEKDAQIYLLKNIYY |
| | GEFLNKFVKNSKVFFKITNEVIKINKQRNQKTGHYKYQKFENI |
| | EKTVPVEYLAIIQSREMINNQDKEEKNTYIDFIQQIFLKGFIDY |
| | LNKNNLKYIESNNNNDNNDIFSKIKIKKDNKEKYDKILKNYE |
| | KHNRNKEIPHEINEFVREIKLGKILKYTENLNMFYLILKLLNH |
| | KELTNLKGSLEKYQSANKEETFSDELELINLLNLDNNRVTEDF |
| | ELEANEIGKFLDFNENKIKDRKELKKFDTNKIYFDGENIIKHR |
| | AFYNIKKYGMLNLLEKIADKAKYKISLKELKEYSNKKNEIEK |
| | NYTMQQNLHRKYARPKKDEKFNDEDYKEYEKAIGNIQKYTH |
| | LKNKVEFNELNLLQGLLLKILHRLVGYTSIWERDLRFRLKGEF |
| | PENHYIEEIFNFDNSKNVKYKSGQIVEKYINFYKELYKDNVEK |
| | RSIYSDKKVKKLKQEKKDLYIANYIAHFNYIPHAEISLLEVLE |
| | NLRKLLSYDRKLKNAIMKSIVDILKEYGFVATFKIGADKKIEI |
| | QTLESEKIVHLKNLKKKKLMTDRNSEELCELVKVMFEYKALE |
| | GSGSGVDTLSGLSSEQGQSGDMTIEEDSATHIKFSKRDEDGKE |
| | LAGATMELRDSSGKTISTWISDGQVKDFYLYPGKYTFVETAA |
| | PDGYEVATAITFTVNEQGQVTVNGKATKGDAHISGLEVLFQG |
| | PGHHHHHHHH* (SEQ ID NO: 31) |
| dLbuCas13a- | MTGMKVTKVGGISHKKYTSEGRLVKSESEENRTDERLSALLN |
| SpyC-3C-His8 | MRLDMYIKNPSSTETKENQKRIGKLKKFFSNKMVYLKDNTLS |
| | LKNGKKENIDREYSETDILESDVRDKKNFAVLKKIYLNENVN |
| | SEELEVFRNDIKKKLNKINSLKYSFEKNKANYQKINENNIEKV |
| | EGKSKRNIIYDYYRESAKRDAYVSNVKEAFDKLYKEEDIAKL |
| | VLEIENLTKLEKYKIREFYHEIIGRKNDKENFAKIIYEEIQNVN |
| | NMKELIEKVPDMSELKKSQVFYKYYLDKEELNDKNIKYAFC |
| | HFVEIEMSQLLKNYVYKRLSNISNDKIKRIFEYQNLKKLIENK |
| | LLNKLDTYVRNCGKYNYYLQDGEIATSDFIARNRQNEAFLRN |
| | IIGVSSVAYFSLRNILETENENDITGRMRGKTVKNNKGEEKYV |
| | SGEVDKIYNENKKNEVKENLKMFYSYDFNMDNKNEIEDFFA |
| | NIDEAISSIAHGIVAFNLELEGKDIFAFKNIAPSEISKKMFQNEI |
| | NEKKLKLKIFRQLNSANVFRYLEKYKILNYLKRTRFEFVNKNI |
| | PFVPSFTKLYSRIDDLKNSLGIYWKTPKTNDDNKTKEIIDAQIY |
| | LLKNIYYGEFLNYFMSNNGNFFEISKEIIELNKNDKRNLKTGF |
| | YKLQKFEDIQEKIPKEYLANIQSLYMINAGNQDEEEKDTYIDFI |
| | QKIFLKGFMTYLANNGRLSLIYIGSDEETNTSLAEKKQEFDKF |
| | LKKYEQNNNIKIPYEINEFLREIKLGNILKYTERLNMFYLILKL |
| | LNHKELTNLKGSLEKYQSANKEEAFSDQLELINLLNLDNNRV |
| | TEDFELEADEIGKFLDFNGNKVKDNKELKKFDTNKIYFDGENI |
| | IKHRAFYNIKKYGMLNLLEKIADKAGYKISIEELKKYSNKKNE |
| | IEKNHKMQENLHRKYARPRKDEKFTDEDYESYKQAIENIEEY |
| | THLKNKVEFNELNLLQGLLLRILHRLVGYTSIWERDLRFRLK |
| | GEFPENQYIEEIFNFENKKNVKYKGGQIVEKYIKFYKELHQND |
| | EVKINKYSSANIKVLKQEKKDLYIANYIAAFNYIPHAEISLLEV |
| | LENLRKLLSYDRKLKNAVMKSVVDILKEYGFVATFKIGADK |
| | KIGIQTLESEKIVHLKNLKKKKLMTDRNSEELCKLVKIMFEYK |
| | MEEKKSENPAGGGLNDIFEAQKIEWHEGSGSGVDTLSGLSSE |
| | QGQSGDMTIEEDSATHIKFSKRDEDGKELAGATMELRDSSGK |
| | TISTWISDGQVKDFYLYPGKYTFVETAAPDGYEVATAITFTVN |
| | EQGQVTVNGKATKGDAHISGLEVLFQGPGHHHHHHHH*(SEQ |
| | ID NO: 32) |
| dEsCas13d-SpyC- | MTGMGKKIHARDLREQRKTDRTEKFADQNKKREAERAVPK |
| 3C-His8 | KDAAVSVKSVSSVSSKKDNVTKSMAKAAGVKSVFAVGNTV |
| | YMTSFGRGNDAVLEQKIVDTSHEPLNIDDPAYQLNVVTMNG |
| | YSVTGHRGETVSAVTDNPLRRFNGRKKDEPEQSVPTDMLCL |
| | KPTLEKKFFGKEFDDNIHIQLIYNILDIEKILAVYSTNAIYALNN |
| | MSADENIENSDFFMKRTTDETFDDFEKKKESTNSREKADFDA |
| | FEKFIGNYRLAYFADAFYVNKKNPKGKAKNVLREDKELYSV |
| | LTLIGKLAHWCVASEEGRAEFWLYKLDELKDDFKNVLDVVY |
| | NRPVEEINNRFIENNKVNIQILGSVYKNTDIAELVRSYYEFLIT |
| | KKYKNMGFSIKKLRESMLEGKGYADKEYDSVRNKLYQMTD |
| | FILYTGYINEDSDRADDLVNTLRSSLKEDDKTTVYCKEADYL |
| | WKKYRESIREVADALDGDNIKKLSKSNIEIQEDKLRKCFISYA |
| | DSVSEFTKLIYLLTRFLSGKEINDLVTTLINKFDNIRSFLEIMDE |
| | LGLDRTFTAEYSFFEGSTKYLAELVELNSFVKSCSFDINAKRT |
| | MYRDALDILGIESDKTEEDIEKMIDNILQIDANGDKKLKKNNG |
| | LRNFIASNVIDSNRFKYLVRYGNPKKIRETAKCKPAVRFVLNE |
| | IPDAQIERYYEACCPKNTALCSANKRREKLADMIAEIKFENFS |
| | DAGNYQKANVTSRTSEAEIKRKNQAIIRLYLTVMYIMLKNLV |
| | NVNARYVIAFHCVERDTKLYAESGLEVGNIEKNKTNLTMAV |
| | MGVKLENGIIKTEFDKSFAENAANRYLRNARWYKLILDNLKK |
| | SERAVVNEFANTVCALNAIRNININIKEIKEVENYFALYHYLIQ |
| | KHLENRFADKKVERDTGDFISKLEEHKTYCKDFVKAYCTPFG |
| | YNLVRYKNLTIDGLFDKNYPGKDDSDEQKGSGSGVDTLSGLS |
| | SEQGQSGDMTIEEDSATHIKFSKRDEDGKELAGATMELRDSS |
| | GKTISTWISDGQVKDFYLYPGKYTFVETAAPDGYEVATAITFT |
| | VNEQGQVTVNGKATKGDAHISGLEVLFQGPGHHHHHHHH* |
| | (SEQ ID NO: 34) |
| Maleimide-spyTag | Maleimide-(CH₂)₃-SGSGAHIVMVDAYKPTK (SEQ ID NO: 35) |
| (synthetic peptide) | |

SpyTag peptides were amino-terminally maleimide-labeled and commercially synthesized (GenScript Biotech, Singapore). Received lyophilized and dissolved in PBS before storage at −20° C.

Guide RNA and Target RNA sequences were commercially synthesized as gBlocks containing a T7 promoter sequence followed by the 23nt-length spacer sequence and the canonical scaffold sequence of the crRNA or direct repeat (DR) sequences and flanked at 3′ by a T7 terminator sequence. The gRNA sequences are shown in Table 5.

TABLE 5
Guide RNA (gRNA) sequences

| Protein Name | gRNA name | gRNA sequence |
|---|---|---|
| His8-3C-SpyC- | SauCas9_sgRNA- | AAGCTTCCTTCCGCGAAATTAATACGACTCA |
| dSauCas9 | hsRPP30 | CTATAGGGCGCGCAGAGCCTTCAGGTCAGAA |
| | | GTTTTAGTACTCTGGAAACAGAATCTACTAA |
| | | AACAAGGCAAAATGCCGTGTTTATCTCGTCA |
| | | ACTTGTTGGCGAGATTTTTACTAGTCCCCCTA |
| | | GCATAACCCCTTGGGGCCTCTAAACGGGTCT |
| | | TGAGGGGTTTTTTGCCCCTGAGAGTGCCAGA |
| | | GACCACACCGGAAAACTTTTCACTTGAAGTA |
| | | TTGTATACGACAGGGTGCGTGTACCTACCAA |
| | | ACCTGTTTAAACTAAGTTCAGACTAGTTGGA |
| | | AGTGTGTGCGTCAATCGAGTTCGTA (SEQ ID |
| | | NO3: 6) |
| | SauCas9_sgRNA- | AAGCTTCCTTCCGCGAAATTAATACGACTCA |
| | SARS2-N | CTATAGGGGTCCACCAAACGTAATGCGGGGT |
| | | GTTTTAGTACTCTGGAAACAGAATCTACTAA |
| | | AACAAGGCAAAATGCCGTGTTTATCTCGTCA |
| | | ACTTGTTGGCGAGATTTTTTAAGAGGTCTCCT |
| | | AATTCAGTCCCCCTAGCATAACCCCTTGGGG |
| | | CCTCTAAACGGGTCTTGAGGGGTTTTTTGCCC |
| | | CTGAGAGGGAGTCCCCACACTACCATCGGAA |
| | | TTCACACCGGAAAACTTTTCACTTGAAGTATT |
| | | GTATACGACAGGGTGCGTGTACCTACCAAAC |
| | | CTGTTTAAACTAAGTTCAGACTAGTTGGAAG |
| | | TGTGTCTAGTTCTTAGTTTTCGTCACTAGAGG |
| | | GCCCACGCTTTATTTTTAT (SEQ ID NO: 37) |
| | SauCas9_sgRNA- | AAGCTTCCTTCCGCGAAATTAATACGACTCA |
| | SARS2-N2 | CTATAGGGCCAATGCGCGACATTCCGAAGAA |
| | | GTTTTAGTACTCTGGAAACAGAATCTACTAA |
| | | AACAAGGCAAAATGCCGTGTTTATCTCGTCA |
| | | ACTTGTTGGCGAGATTTTTTAAGAGGTCTCCT |
| | | AATTCAGTCCCCCTAGCATAACCCCTTGGGG |
| | | CCTCTAAACGGGTCTTGAGGGGTTTTTTGCCC |
| | | CTGAGAGGGAGTCCCCACACTACCATCGGAA |
| | | TTCACACCGGAAAACTTTTCACTTGAAGTATT |
| | | GTATACGACAGGGTGCGTGTACCTACCAAAC |
| | | CTGTTTAAACTAAGTTCAGACTAGTTGGAAG |
| | | TGTGTCTAGTTCTTAGTTTTCGTCACTAGAGG |
| | | GCCCACGCTTTATTTTTAT (SEQ ID NO: 38) |
| | SauCas9_sgRNA- | AAGCTTCCTTCCGCGAAATTAATACGACTCA |
| | SARS2-N3 | CTATAGGGGTTTTGTATGCGTCAATATGCTTG |
| | | TTTTAGTACTCTGGAAACAGAATCTACTAAA |
| | | ACAAGGCAAAATGCCGTGTTTATCTCGTCAA |
| | | CTTGTTGGCGAGATTTTTTAAGAGGTCTCCTA |
| | | ATTCAGTCCCCCTAGCATAACCCCTTGGGGC |
| | | CTCTAAACGGGTCTTGAGGGGTTTTTTGCCCC |
| | | TGAGAGGGAGTCCCCACACTACCATCGGAAT |
| | | TCACACCGGAAAACTTTTCACTTGAAGTATT |
| | | GTATACGACAGGGTGCGTGTACCTACCAAAC |
| | | CTGTTTAAACTAAGTTCAGACTAGTTGGAAG |
| | | TGTGTCTAGTTCTTAGTTTTCGTCACTAGAGG |
| | | GCCCACGCTTTATTTTTAT (SEQ ID NO: 39) |
| | SauCas9_sgRNA- | AAGCTTCCTTCCGCGAAATTAATACGACTCA |
| | SARS2-E | CTATAGGGAATATTGCAGCAGTACGCACACA |
| | | GTTTTAGTACTCTGGAAACAGAATCTACTAA |
| | | AACAAGGCAAAATGCCGTGTTTATCTCGTCA |
| | | ACTTGTTGGCGAGATTTTTTAAGAGGTCTCCT |
| | | AATTCAGTCCCCCTAGCATAACCCCTTGGGG |
| | | CCTCTAAACGGGTCTTGAGGGGTTTTTTGCCC |
| | | CTGAGAGGGAGTCCCCACACTACCATCGGAA |
| | | TTCACACCGGAAAACTTTTCACTTGAAGTATT |
| | | GTATACGACAGGGTGCGTGTACCTACCAAAC |
| | | CTGTTTAAACTAAGTTCAGACTAGTTGGAAG |
| | | TGTGTCTAGTTCTTAGTTTTCGTCACTAGAGG |
| | | GCCCACGCTTTATTTTTAT (SEQ ID NO: 40) |
| | SauCas9_sgRNA- | AAGCTTCCTTCCGCGAAATTAATACGACTCA |
| | SARS2-E2 | CTATAGGGAGCGCAGTAAGGATGGCTAGTGT |
| | | GTTTTAGTACTCTGGAAACAGAATCTACTAA |
| | | AACAAGGCAAAATGCCGTGTTTATCTCGTCA |
| | | ACTTGTTGGCGAGATTTTTTAAGAGGTCTCCT |
| | | AATTCAGTCCCCCTAGCATAACCCCTTGGGG |
| | | CCTCTAAACGGGTCTTGAGGGGTTTTTTGCCC |
| | | CTGAGAGGGAGTCCCCACACTACCATCGGAA |
| | | TTCACACCGGAAAACTTTTCACTTGAAGTATT |
| | | GTATACGACAGGGTGCGTGTACCTACCAAAC |
| | | CTGTTTAAACTAAGTTCAGACTAGTTGGAAG |
| | | TGTGTCTAGTTCTTAGTTTTCGTCACTAGAGG |
| | | GCCCACGCTTTATTTTTAT (SEQ ID NO: 41) |
| dLwaCas13a- | LwaCas13a_ | AAGCTTCCTTCCGCGAAATTAATACGACTCA |
| SpyC-3C-His8 | sgRNA-hsRPP30 | CTATAGGGGATTTAGACTACCCCAAAAACGA |
| | | AGGGGACTAAAACCGCGCAGAGCCTTCAGGT |
| | | CAGAATAAGAGGTCTCCTAATTCAGTCCCCC |
| | | TAGCATAACCCCTTGGGGCCTCTAAACGGGT |
| | | CTTGAGGGGTTTTTTGCCCCTGAGAGGGAGT |
| | | CCCCACACTACCATCGGAATTCACACCGGAA |
| | | AACTTTTCACTTGAAGTATTGTATACGACAG |
| | | GGTGCGTGTACCTACCAAACCTGTTTAAACT |
| | | AAGTTCAGACTAGTTGGAAGTGTGTCTAGTT |
| | | CTTAGTTTTCGTCACTAGAGGGCCCACGCTTT |
| | | ATTTTTAT (SEQ ID NO: 42) |
| | LwaCas13a_ | AAGCTTCCTTCCGCGAAATTAATACGACTCA |
| | sgRNA-SARS2- | CTATAGGGGATTTAGACTACCCCAAAAACGA |
| | N | AGGGGACTAAAACGTCCACCAAACGTAATGC |
| | | GGGGTTAAGAGGTCTCCTAATTCAGTCCCCC |
| | | TAGCATAACCCCTTGGGGCCTCTAAACGGGT |
| | | CTTGAGGGGTTTTTTGCCCCTGAGAGGGAGT |
| | | CCCCACACTACCATCGGAATTCACACCGGAA |
| | | AACTTTTCACTTGAAGTATTGTATACGACAG |
| | | GGTGCGTGTACCTACCAAACCTGTTTAAACT |
| | | AAGTTCAGACTAGTTGGAAGTGTGTCTAGTT |
| | | CTTAGTTTTCGTCACTAGAGGGCCCACGCTTT |
| | | ATTTTTAT (SEQ ID NO: 43) |
| | LwaCas13a_ | AAGCTTCCTTCCGCGAAATTAATACGACTCA |
| | sgRNA-SARS2- | CTATAGGGGATTTAGACTACCCCAAAAACGA |
| | N2 | AGGGGACTAAAACCCAATGCGCGACATTCCG |
| | | AAGAATAAGAGGTCTCCTAATTCAGTCCCCC |
| | | TAGCATAACCCCTTGGGGCCTCTAAACGGGT |
| | | CTTGAGGGGTTTTTTGCCCCTGAGAGGGAGT |
| | | CCCCACACTACCATCGGAATTCACACCGGAA |
| | | AACTTTTCACTTGAAGTATTGTATACGACAG |
| | | GGTGCGTGTACCTACCAAACCTGTTTAAACT |
| | | AAGTTCAGACTAGTTGGAAGTGTGTCTAGTT |
| | | CTTAGTTTTCGTCACTAGAGGGCCCACGCTTT |
| | | ATTTTTAT (SEQ ID NO: 44) |
| | LwaCas13a_ | AAGCTTCCTTCCGCGAAATTAATACGACTCA |
| | sgRNA-SARS2- | CTATAGGGGATTTAGACTACCCCAAAAACGA |
| | N3 | AGGGGACTAAAACGTTTTGTATGCGTCAATA |
| | | TGCTTTAAGAGGTCTCCTAATTCAGTCCCCCT |
| | | AGCATAACCCCTTGGGGCCTCTAAACGGGTC |
| | | TTGAGGGGTTTTTTGCCCCTGAGAGGGAGTC |
| | | CCCACACTACCATCGGAATTCACACCGGAAA |
| | | ACTTTTCACTTGAAGTATTGTATACGACAGG |
| | | GTGCGTGTACCTACCAAACCTGTTTAAACTA |
| | | AGTTCAGACTAGTTGGAAGTGTGTCTAGTTC |
| | | TTAGTTTTCGTCACTAGAGGGCCCACGCTTTA |
| | | TTTTTAT (SEQ ID NO: 45) |
| | LwaCas13a_ | AAGCTTCCTTCCGCGAAATTAATACGACTCA |
| | sgRNA-SARS2-E | CTATAGGGGATTTAGACTACCCCAAAAACGA |
| | | AGGGGACTAAAACAATATTGCAGCAGTACGC |
| | | ACACATAAGAGGTCTCCTAATTCAGTCCCCC |
| | | TAGCATAACCCCTTGGGGCCTCTAAACGGGT |
| | | CTTGAGGGGTTTTTTGCCCCTGAGAGGGAGT |
| | | CCCCACACTACCATCGGAATTCACACCGGAA |
| | | AACTTTTCACTTGAAGTATTGTATACGACAG |
| | | GGTGCGTGTACCTACCAAACCTGTTTAAACT |
| | | AAGTTCAGACTAGTTGGAAGTGTGTCTAGTT |
| | | CTTAGTTTTCGTCACTAGAGGGCCCACGCTTT |
| | | ATTTTTAT (SEQ ID NO: 46) |
| | LwaCas13a_ | AAGCTTCCTTCCGCGAAATTAATACGACTCA |
| | sgRNA-SARS2- | CTATAGGGGATTTAGACTACCCCAAAAACGA |
| | E2 | AGGGGACTAAAACAGCGCAGTAAGGATGGC |
| | | TAGTGTTAAGAGGTCTCCTAATTCAGTCCCCC |
| | | TAGCATAACCCCTTGGGGCCTCTAAACGGGT |
| | | CTTGAGGGGTTTTTTGCCCCTGAGAGGGAGT |
| | | CCCCACACTACCATCGGAATTCACACCGGAA |
| | | AACTTTTCACTTGAAGTATTGTATACGACAG |
| | | GGTGCGTGTACCTACCAAACCTGTTTAAACT |
| | | AAGTTCAGACTAGTTGGAAGTGTGTCTAGTT |
| | | CTTAGTTTTCGTCACTAGAGGGCCCACGCTTT |
| | | ATTTTTAT (SEQ ID NO: 47) |
| dLbuCas13a- | LbuCas13a_ | AAGCTTCCTTCCGCGAAATTAATACGACTCA |
| SpyC-3C-His8 | sgRNA-hsRPP30 | CTATAGGGGGCCACCCCAAAAATGAAGGGG |
| | | ACTAAAACACGCGCAGAGCCTTCAGGTCAGA |
| | | ATAAGAGGTCTCCTAATTCAGTCCCCCTAGC |
| | | ATAACCCCTTGGGGCCTCTAAACGGGTCTTG |
| | | AGGGGTTTTTTGCCCCTGAGAGGGAGTCCCC |
| | | ACACTACCATCGGAATTCACACCGGAAAACT |
| | | TTTCACTTGAAGTATTGTATACGACAGGGTG |
| | | CGTGTACCTACCAAACCTGTTTAAACTAAGT |
| | | TCAGACTAGTTGGAAGTGTGTCTAGTTCTTA |
| | | GTTTTCGTCACTAGAGGGCCCACGCTTTATTT |
| | | TTAT (SEQ ID NO: 48) |
| | LbuCas13a_ | AAGCTTCCTTCCGCGAAATTAATACGACTCA |
| | sgRNA-SARS2- | CTATAGGGGGCCACCCCAAAAATGAAGGGG |
| | N | ACTAAAACAGTCCACCAAACGTAATGCGGGG |
| | | TTAAGAGGTCTCCTAATTCAGTCCCCCTAGC |
| | | ATAACCCCTTGGGGCCTCTAAACGGGTCTTG |
| | | AGGGGTTTTTTGCCCCTGAGAGGGAGTCCCC |
| | | ACACTACCATCGGAATTCACACCGGAAAACT |
| | | TTTCACTTGAAGTATTGTATACGACAGGGTG |
| | | CGTGTACCTACCAAACCTGTTTAAACTAAGT |
| | | TCAGACTAGTTGGAAGTGTGTCTAGTTCTTA |
| | | GTTTTCGTCACTAGAGGGCCCACGCTTTATTT |
| | | TTAT (SEQ ID NO: 49) |
| | LbuCas13a_ | AAGCTTCCTTCCGCGAAATTAATACGACTCA |
| | sgRNA-SARS-2- | CTATAGGGGGCCACCCCAAAAATGAAGGGG |
| | N2 | ACTAAAACACCAATGCGCGACATTCCGAAGA |
| | | ATAAGAGGTCTCCTAATTCAGTCCCCCTAGC |
| | | ATAACCCCTTGGGGCCTCTAAACGGGTCTTG |
| | | AGGGGTTTTTTGCCCCTGAGAGGGAGTCCCC |
| | | ACACTACCATCGGAATTCACACCGGAAAACT |
| | | TTTCACTTGAAGTATTGTATACGACAGGGTG |
| | | CGTGTACCTACCAAACCTGTTTAAACTAAGT |
| | | TCAGACTAGTTGGAAGTGTGTCTAGTTCTTA |
| | | GTTTTCGTCACTAGAGGGCCCACGCTTTATTT |
| | | TTAT (SEQ ID NO: 50) |
| | LbuCas13a_ | AAGCTTCCTTCCGCGAAATTAATACGACTCA |
| | sgRNA-SARS-2- | CTATAGGGGGCCACCCCAAAAATGAAGGGG |
| | N3 | ACTAAAACAGTTTTGTATGCGTCAATATGCTT |
| | | TAAGAGGTCTCCTAATTCAGTCCCCCTAGCA |
| | | TAACCCCTTGGGGCCTCTAAACGGGTCTTGA |
| | | GGGGTTTTTTGCCCCTGAGAGGGAGTCCCCA |
| | | CACTACCATCGGAATTCACACCGGAAAACTT |
| | | TTCACTTGAAGTATTGTATACGACAGGGTGC |
| | | GTGTACCTACCAAACCTGTTTAAACTAAGTT |
| | | CAGACTAGTTGGAAGTGTGTCTAGTTCTTAG |
| | | TTTTCGTCACTAGAGGGCCCACGCTTTATTTT |
| | | TAT (SEQ ID NO: 51) |
| | LbuCas13a_ | AAGCTTCCTTCCGCGAAATTAATACGACTCA |
| | sgRNA-SARS2-E | CTATAGGGGGCCACCCCAAAAATGAAGGGG |
| | | ACTAAAACAAATATTGCAGCAGTACGCACAC |
| | | ATAAGAGGTCTCCTAATTCAGTCCCCCTAGC |
| | | ATAACCCCTTGGGGCCTCTAAACGGGTCTTG |
| | | AGGGGTTTTTTGCCCCTGAGAGGGAGTCCCC |
| | | ACACTACCATCGGAATTCACACCGGAAAACT |
| | | TTTCACTTGAAGTATTGTATACGACAGGGTG |
| | | CGTGTACCTACCAAACCTGTTTAAACTAAGT |
| | | TCAGACTAGTTGGAAGTGTGTCTAGTTCTTA |
| | | GTTTTCGTCACTAGAGGGCCCACGCTTTATTT |
| | | TTAT (SEQ ID NO: 52) |
| | LbuCas13a_ | AAGCTTCCTTCCGCGAAATTAATACGACTCA |
| | sgRNA-SARS2- | CTATAGGGGGCCACCCCAAAAATGAAGGGG |
| | E2 | ACTAAAACAAGCGCAGTAAGGATGGCTAGT |
| | | GTTAAGAGGTCTCCTAATTCAGTCCCCCTAG |
| | | CATAACCCCTTGGGGCCTCTAAACGGGTCTT |
| | | GAGGGGTTTTTTGCCCCTGAGAGGGAGTCCC |
| | | CACACTACCATCGGAATTCACACCGGAAAAC |
| | | TTTTCACTTGAAGTATTGTATACGACAGGGT |
| | | GCGTGTACCTACCAAACCTGTTTAAACTAAG |
| | | TTCAGACTAGTTGGAAGTGTGTCTAGTTCTTA |
| | | GTTTTCGTCACTAGAGGGCCCACGCTTTATTT |
| | | TTAT (SEQ ID NO: 53) |
| dEsCas13d- | EsCas13d_ | AAGCTTCCTTCCGCGAAATTAATACGACTCA |
| SpyC-3C-His8 | sgRNA-hsRPP30 | CTATAGGGAACTACACCCGTGCAAAAATGCA |
| | | GGGGTCTAAAACCGCGCAGAGCCTTCAGGTC |
| | | AGAATAAGAGGTCTCCTAATTCAGTCCCCCT |
| | | AGCATAACCCCTTGGGGCCTCTAAACGGGTC |
| | | TTGAGGGGTTTTTTGCCCCTGAGAGGGAGTC |
| | | CCCACACTACCATCGGAATTCACACCGGAAA |
| | | ACTTTTCACTTGAAGTATTGTATACGACAGG |
| | | GTGCGTGTACCTACCAAACCTGTTTAAACTA |
| | | AGTTCAGACTAGTTGGAAGTGTGTCTAGTTC |
| | | TTAGTTTTCGTCACTAGAGGGCCCACGCTTTA |
| | | TTTTTAT (SEQ ID NO: 54) |
| | EsCas13d_ | AAGCTTCCTTCCGCGAAATTAATACGACTCA |
| | sgRNA-SARS2-N | CTATAGGGAACTACACCCGTGCAAAAATGCA |
| | | GGGGTCTAAAACGTCCACCAAACGTAATGCG |
| | | GGGTTAAGAGGTCTCCTAATTCAGTCCCCCT |
| | | AGCATAACCCCTTGGGGCCTCTAAACGGGTC |
| | | TTGAGGGGTTTTTTGCCCCTGAGAGGGAGTC |
| | | CCCACACTACCATCGGAATTCACACCGGAAA |
| | | ACTTTTCACTTGAAGTATTGTATACGACAGG |
| | | GTGCGTGTACCTACCAAACCTGTTTAAACTA |
| | | AGTTCAGACTAGTTGGAAGTGTGTCTAGTTC |
| | | TTAGTTTTCGTCACTAGAGGGCCCACGCTTTA |
| | | TTTTTAT (SEQ ID NO: 55) |
| | EsCas13d_ | AAGCTTCCTTCCGCGAAATTAATACGACTCA |
| | sgRNA-SARS2-N2 | CTATAGGGAACTACACCCGTGCAAAAATGCA |
| | | GGGGTCTAAAACCCAATGCGCGACATTCCGA |
| | | AGAATAAGAGGTCTCCTAATTCAGTCCCCCT |
| | | AGCATAACCCCTTGGGGCCTCTAAACGGGTC |
| | | TTGAGGGGTTTTTTGCCCCTGAGAGGGAGTC |
| | | CCCACACTACCATCGGAATTCACACCGGAAA |
| | | ACTTTTCACTTGAAGTATTGTATACGACAGG |
| | | GTGCGTGTACCTACCAAACCTGTTTAAACTA |
| | | AGTTCAGACTAGTTGGAAGTGTGTCTAGTTC |
| | | TTAGTTTTCGTCACTAGAGGGCCCACGCTTTA |
| | | TTTTTAT (SEQ ID NO: 56) |
| | EsCas13d_ | AAGCTTCCTTCCGCGAAATTAATACGACTCA |
| | sgRNA-SARS2-N3 | CTATAGGGAACTACACCCGTGCAAAAATGCA |
| | | GGGGTCTAAAACGTTTTGTATGCGTCAATAT |
| | | GCTTTAAGAGGTCTCCTAATTCAGTCCCCCTA |
| | | GCATAACCCCTTGGGGCCTCTAAACGGGTCT |
| | | TGAGGGGTTTTTTGCCCCTGAGAGGGAGTCC |
| | | CCACACTACCATCGGAATTCACACCGGAAAA |
| | | CTTTTCACTTGAAGTATTGTATACGACAGGGT |
| | | GCGTGTACCTACCAAACCTGTTTAAACTAAG |
| | | TTCAGACTAGTTGGAAGTGTGTCTAGTTCTTA |
| | | GTTTTCGTCACTAGAGGGCCCACGCTTTATTT |
| | | TTAT (SEQ ID NO: 57) |
| | EsCas13d_ | AAGCTTCCTTCCGCGAAATTAATACGACTCA |
| | sgRNA-SARS2-E | CTATAGGGAACTACACCCGTGCAAAAATGCA |
| | | GGGGTCTAAAACAATATTGCAGCAGTACGCA |
| | | CACATAAGAGGTCTCCTAATTCAGTCCCCCT |
| | | AGCATAACCCCTTGGGGCCTCTAAACGGGTC |
| | | TTGAGGGGTTTTTTGCCCCTGAGAGGGAGTC |
| | | CCCACACTACCATCGGAATTCACACCGGAAA |
| | | ACTTTTCACTTGAAGTATTGTATACGACAGG |
| | | GTGCGTGTACCTACCAAACCTGTTTAAACTA |
| | | AGTTCAGACTAGTTGGAAGTGTGTCTAGTTC |
| | | TTAGTTTTCGTCACTAGAGGGCCCACGCTTTA |
| | | TTTTTAT (SEQ ID NO: 58) |
| | EsCas13d_ | AAGCTTCCTTCCGCGAAATTAATACGACTCA |
| | sgRNA-SARS2-E2 | CTATAGGGAACTACACCCGTGCAAAAATGCA |
| | | GGGGTCTAAAACAGCGCAGTAAGGATGGCT |
| | | AGTGTTAAGAGGTCTCCTAATTCAGTCCCCCT |
| | | AGCATAACCCCTTGGGGCCTCTAAACGGGTC |
| | | TTGAGGGGTTTTTTGCCCCTGAGAGGGAGTC |
| | | CCCACACTACCATCGGAATTCACACCGGAAA |
| | | ACTTTTCACTTGAAGTATTGTATACGACAGG |
| | | GTGCGTGTACCTACCAAACCTGTTTAAACTA |
| | | AGTTCAGACTAGTTGGAAGTGTGTCTAGTTC |
| | | TTAGTTTTCGTCACTAGAGGGCCCACGCTTTA |
| | | TTTTTAT (SEQ ID NO: 59) |

### Bioinformatic Evaluation of Spacer Sequences

Bioinformatics analysis was performed on a collection of 36 probes and primers coming from RT-qPCR standard protocols and literature review. First, the position and frequency of Single nucleotide polymorphisms (SNPs) against the SARS-Cov2 genome determined the primers with better chances to bind to this virus. Then, primers cross-reference was assessed by alignment against the human genome as well as common human coronaviruses: MERS (MK129253.1), SARS-Cov1 (NC_004718.3), HCoV-229E (NC)002645.1), Hepatitis B (NC_003977.2), HIV1 (NC_001802.1), NL63 (NC_005831.2) and OC43 (AY585228.1).

### Protein Expression and Purification

Bacterial expression plasmids were transformed into Rosetta 2 (DE3) competent cells (Millipore). One colony was inoculated into 2×YT media enriched with 1% glucose, Kanamycin (50 μg/mL), and Chloramphenicol (34 μg/mL) for starter culture and then seeded to 1 L 2×YT production media equally enriched. Protein expression was induced with 400 μM IPTG at 18° C. for 18 h. Cell lysis was performed in cell disruptor at 20 kPsi. The lysate was clarified at 45,000 rpm for 30 min. The supernatant was processed on an ÄKTA FPLC (GE Healthcare), first by affinity purification using a HisTrap column (GE Healthcare) from which protein of interest was eluted with 80-240 mM Imidazole. Fractions then were pooled before further purification by cation exchange with HiTrap Heparin column (GE Healthcare). After elution with nM NaCl, fractions were pooled and concentrated with a 50K amicon ultra centrifugal filter unit. Further purification by gel filtration was performed on a high load Superdex 200 into 20 mM HEPES-NaOH (pH 7.5), 500 mM NaCl, 1 mM TCEP, 5% glycerol. After spin concentration, aliquots were snap-frozen in liquid nitrogen and stored at −80° C.

### RNA Production

Guide RNA and target RNA were transcribed using the NEB HiScribe T7 High Yield RNA Synthesis Kit according to manufacturer protocol with 2.5% Cy5-UTP for Cy5 randomly labeled nucleic acids. Transcription products were purified following the directions of the RNAeasy Kit, resuspended in double distilled water and stored at −20° C. RNA quality was visualized by denaturing gel electrophoresis (100 V for 1 h). The nucleic acid concentration was determined by absorbance (A260 nm) with NanoDrop one.

### SEC-MALS

Purified proteins were injected by an Agilent HPLC 1100 at 3 mg/ml and separated by size within a Superdex 200 10/300 column previously equilibrated in SEC-MALS buffer (20 mM HEPES-NaOH (pH 7.5), 500 mM NaCl, 1 mM TCEP) before entering the sample compartments of the DAWN-EOS multi-angle laser light scattering detector and the Optilab-DSP relative refractive interferometer (Wyatt). Astra software was used for peak alignment and band broadening correction between UV, MALS, and RWe detectors.

### Binary Complex Assembly and Precipitation Assay

Guide RNA was warmed up to 70° C. for 5 min immediately after thawing. Complex formation was performed at 1:1 ratio protein to guide RNA in complex buffer at room temperature for 1 h to a final 5 μM.

### EMSA

Binding reactions were set up in RNAse-free tubes where binding buffer was distributed and Cas: guide RNA complex was serially diluted (2×DF) to a final volume of 30 μL. Cy5 labeled target or non-target previously warmed at 75° C. for 5 min. were spiked in at a constant concentration. Binding reaction was incubated for 1 h at room temperature. Samples were diluted with nondenaturing loading buffer, loaded into 3% (1×TAE or TBE) agarose gel, and run at 4° C. at 100 V for 2 h Gel imaging was performed with the iBright FL1500 imaging system (ThermoFisher) with the fluorescent imaging protocol (Exc. 610-660 nm, Em. 710-730 nm).

### MST

The short RNA targets were commercially synthesized by Integrated DNA technologies (IDT, Inc.) and designed to contain a single-nucleotide-labeled with Cy5 at the 5′ end. Whereas in vitro transcribed targets were randomly labeled with Cy5-UTP integration during transcription by including a mix (2.5:10) of UTP Cy5 labelled/unlabeled nucleotide in the IVT reaction. The previously assembled complex Cas: guide RNA was serially diluted in binding buffer, and target at constant concentration was later pipetted in. The reaction was incubated at room temperature in the dark for 1 h. Measurements were performed using the PicoRed laser of Nano Temper MST instrument at 40% LED power, Medium MST power for short targets and positive IVT target, and 5% LED power with Medium MST power for IVT negative control in order to reach 20,000 counts at 250 pM in all cases. In total, 16 concentrations with three replicates each were analyzed per binding test.

### OECT Fabrication

OECTs were fabricated using photolithography and Parylene-C peel-off techniques. Briefly, a layer of photoresist (AZ5214) is spin coated on a glass substrate and treated with UV light using a mask aligner. After sputter coating the substrate with 10 nm of Cr and 100 nm of Au, a standard lift-off process was performed in hot DMSO to remove the UV exposed region. Then, the second layer of photoresist (AZ9260) was spin coated on the same substrate to insulate the gold connection pads, followed with the Parylene-C peel-off. The OECT channels (a length of 10 μm in and width of 100 μm) was formed by reactive ion etching technique. The p-type accumulation mode organic film, (p(g0T2-g6T2)), was dissolved in a chloroform solution (5 g/L) and then, was spin coated (800 rpm, 45 s) on the substrates to yield a film thickness of about 70 nm in the channel.

The gate electrodes were fabricated on 175 μm-thick Kapton (polyimide) substrates. The Kapton substrates were sputter coated with 10 nm of Cr and 100 nm of Au and were patterned using a craft cutter to yield a final form. A square geometry at the tip of the electrode defines the sensor active area (0.8×0.8 mm). The electrodes were electrochemically cleaned in 10 mM sulfuric acid (H2SO4) using cyclic voltammetry (CV). The potential is scanned from −0.2 V to 1.2 V twenty times at the scan rate 100 mV s−1.

Quartz Crystal Microbalance with Dissipation Monitoring (QCM-D)

Performed by collaborators from Prof. Inal's lab. QCM-D measurements were carried out using a Q-sense analyzer (QE401, Biolin Scientific). The piezoelectrically active gold sensor (0.7854 cm²) was pre-coated with 1,6-hexanedithiol (HDT) self-assembled monolayer (SAM) and then placed into the QCM-D setup. After the stabilization of frequency (Δf) and dissipation (AD) in PBS, the peptide solution (0.1 mg/mL SpyTag peptide in PBS) was pumped into the fluidic chamber with a flow rate of 100 μL/min. After the full coverage of sensor surface with the solution, the pump was stopped to modify the surface of sensor in a stationary mode. After incubating the sensor for an hour in the peptide solution, the sensor surface was rinsed with PBS for 15 min to remove the excess amount of peptides from the surface. The same procedure was conducted to expose the surface complex protein (Cas:guide RNA) at 5 μM in binding buffer. The 7th overtone was selected to present the QCM-D data. The change in the mass and thickness during the functionalization was calculated from the Sauerbrey equation (1):

$$Δ m = ( - 17.7 ) / n Δf\_n \qquad ( 1 )$$

where n is the overtone number (−17.7) which was calculated considering the resonant frequency, active area, density and shear modulus of the quartz crystal sensor. The mass and thickness of the each bio-chemical layer is extracted from the following equation by using their molecular weight.

$$ds = Δ m / Ar 100 \qquad ( 2 )$$

where Δm is the change in mass (ng), A is the area (cm2), and r is the estimated density of the layer (g cm−3, assumed same as water which is taken as 1 g cm−3).

### Electrochemical Impedance Spectroscopy (EIS) and Cyclic Voltammetry (CV)

Formation of bio-chemical layers on the gold electrode were examined using electrochemical impedance spectroscopy (EIS) and cyclic voltammetry (CV) techniques. A three-electrode setup was connected to a potentiostat (Autolab PGstat128N with Nova software, MetroOhm) for both measurements. A platinum wire and an Ag/AgCl electrode were used as the counter and reference electrodes, respectively. The gold gate electrode (working electrode) was immersed in in 5 mL of 10 mM PBS (pH 7.4) containing 10 mM of [Fe(CN)6]3−/4− for the measurements. For CV measurements, the potential window was scanned from −0.2 V and 0.5 V at the scan rate of 100 mV s−1. The impedance spectra were measured at a zero DC offset versus open circuit potential and an alternating current (AC) modulation of 10 mV over a frequency range of 0.1-100000 Hz. The data was analyzed using Nova software using appropriate equivalent circuit modelling.

### Gate Biofunctionalization

The HDT-SAM solution was prepared in 100% ethanol containing 1 mM of HDT. The gold electrodes were immersed in this solution for an hour, followed with rinsing in ethanol and dried under a Nitrogen 128. Next, the electrodes were modified with the synthetic Maleimide-modified Spy Tag peptide (0.1 mg/mL) in PBS by incubating an hour. After rinsing the electrodes in PBS, they were exposed to the distinct complex proteins (Cas: guide RNA) in binding buffer for one hour and then rinsed with PBS. The functionalized gate electrodes were used at the same day and stored them in an ice box during the measurements to prevent from any degradation issues.

### Sensor Operation

The steady-state characteristics (IV) of the transistor were measured using a Keithley 2602A Source Meter Unit operated by a customized

LabVIEW software. The drain (VD) and gate (VG) voltages were applied while the source electrode functioned as the common ground in both circuits. The steady-state measurements of the p(g0T2-g6T2)-based OECTs were conducted by varying VG (0.2-0.6 V, step 0.05 V) and VD (0 to −0.6 V, step of 0.05 V), and the drain current (ID) was obtained simultaneously.

A PDMS well (1 cm diameter, 2 mm thick) is placed on top of the OECT and it is filled with 100 μL of PBS to perform IV measurements. After functionalization steps, the reference (blank) response of the sensor is obtained by immersing the gate electrode into the PBS. The same electrode was then incubated for 10 mins with a 5 μL drop of binding buffer containing proteins, rinsed in the buffer and twice in PBS (phosphate-buffered saline, pH 7.4, ionic strength 0.162 M), and then mounted on top of the channel in a parallel fashion to complete the OECT biosensor.

### Results

### Sensor Design: Mechanism of Action and Features

Studies sought to integrate the high specificity and programmable features of RNA-targeting Cas systems with the high sensitivity of Organic Electrochemical Transistors (OECT) to rapidly (<15 min), sensitively and selectively detect RNA in raw samples. The sensor includes a biological recognition unit made of three building blocks: a maleimide modified Spy Tag-peptide, a SpyCatcher-fused Cas protein, and a guide RNA (gRNA). Additionally, various versions (long and short) of the sequences were produced and used as target or negative controls [data not shown].

The Spy Tag/SpyCatcher system, derived from the Streptococcus pyrogenes fibronectin-binding protein FbaB, spontaneously and irreversibly forms an isopeptide bond under diverse conditions (i.e., pH, buffer and temperature). Its robustness and binding formation speed (minutes) facilitated the biofunctionalization of the disclosed receptor modules over the gate electrode.

A flexible linker composed of glycine-serine amino acids was selected to dynamically separate the Cas module from the anchoring SpyCatcher. With this flexibility the aim is to increase the packing density on the gate surface while reducing steric clashes between close receptor units.

The ribonucleoprotein complex Cas:gRNA is preassembled in solution. Then the receptor module is biofunctionalized on top of the gold gate electrode in three phasessteps. First, a 1,6-hexanedithiol (HDT) self-assembled monolayer (SAM) is formed on top of the gold electrode. Second, the maleimide-modified Spy Tag peptide is chemically immobilized, generating a chem-SAM layer. Last, the Cas receptor module is coupled through the Spy Tag/SpyCatcher isopeptide-covalent-binding system.

After biofunctionalization of the gate, the sensor works in three steps. 1) Sample incubation (10 min) on top of the gate electrode with gentle mixing by pipetting up and down, 2) In order to increase specificity, the gate electrode is washed with binding or washing buffer and with PBS to detach non-specific binders, 3) The gate is placed on top of the OECT base to complete the transistor setup and the electrical response is measured in PBS.

### Selection of the Best Recognition Unit (Cas Protein) and Target Sequence to Detect SARS-Cov 2

There are numerous Cas proteins reported in the literature. A 5-step path was set up to select the top four Cas effectors that best fulfill sensor needs: 1) Retain only Cas effectors capable of directly targeting RNA. 2) Filter out Cas effectors that require a protospacer adjacent motif (PAM) or protospacer flanking site (PFS). 3) Prioritize effectors with in vitro experimental characterization, 4) Select Cas proteins for which guide RNA sequences and features have been reported, and 5) Prioritize Cas effectors with available kinetic and mechanistic information.

Four Cas effectors were selected with this strategy: a) Staphylococcus aureus (SauCas9), b) Leptotrichia wadeii. (LwaCas13a), c) Leptotrichia buccalis. (LbuCas13a) and d) Eubacterium siraeum (EsCas13d). These four Cas effectors are class 2 which means that they work independently of other proteins but perform different functions with their multiple domains. LbuCas13a, LwaCas13a and EsCas13d belong to the Cas13 family that targets RNA, possess collateral RNA cleavage activity, and a HEPN-dimer nuclease cleavage motif 135-137. In contrast, SauCas9 can bind and cleave both DNA and RNA and possess two different nuclease domains (RuvC and HNH). The nuclease LwaCas13a is mostly known for its use in the SHERLOCK system and, unlike the other selected effectors, does not have a published structure. EsCas13d is the smallest of the effectors selected and the only one from the subtype d of the Cas13 family. In one report, this protein showed better activity in vitro than inside mammalian cells. Fortunately, the modularity of the disclosed sensing platform allows for the easy interchange and assessment of those four receptor units under similar conditions.

The next step was to design appropriate guide RNA sequences for each Cas effector. The aim was for the sensor to detect the SARS-Cov 2 isolate Wuhan-Hu 1 [Accession number: NC_045512.2]. To select the regions on the viral genome to be targeted, SARS-Cov-2 targeting primer and probe sequences reported in the literature and those used by standardized RT-qPCR protocols were collected. Considering the dependency of Cas protein to bind on targets with low secondary structure, each reported primer sequence was evaluated against its status in a published SARS-Cov2 RNA secondary structure predictor. To ensure selectivity the spacer sequences moreover should have a low probability of binding to human genes or other human-related viruses. Bioinformatic analysis and filtering of all possible spacer sequences considering their conservation within SARS-CoV-2 genomes and difference with human genome or viral sequences, were performed.

The six best spacer sequences and the four top mentioned Cas proteins were used for further experiments. Because the goal was to detect direct binding of the RNP complex to the RNA target, catalytically dead versions of the selected Cas proteins were designed and produced for these experiments. Three of the studied guide RNAs (N1, N2, and N3) target different regions of the SARS-Cov2 N gene, two guide RNAs (E1 and E2) target two different regions of the SARS-Cov2 gene E, and one guide RNA (RPP30) (designed as a control) targets one region of the human ribonuclease RPP30. All 24 guide RNAs were constructed according to the direct repeat (DR) or CRISPR RNA (crRNA) reported in the literature for the respective Cas to which the 23-nt-length spacer sequence was added.

### Production, Characterization, and Stabilization of the Recognition Unit

All four catalytically dead (dCas) effectors were expressed in E. coli, and purified. Further characterization was pursued by size-exclusion chromatography coupled to multi-angle light scattering (SEC-MALS) [data not shown]. This analysis returned single peaks for three of four effectors with molecular weights in the expected range (<|10%| error). The only exception was dLwaCas13a which showed two peaks with about 20% difference in mass between them and −14% mass difference of the closest peak to the expected molecular weight (which only represents ˜30% of the total mass). This mass value is consistent with impurities present at similar molecular weight as observed in the SDS-PAGE analysis. The SDS-PAGE also showed significant impurities for dSauCas9 which however where not apparent under the native conditions of the SEC-MALS chromatogram.

The disclosed system requires two different types of RNA: guide RNA and target RNA. The guide RNA was mainly in vitro transcribed (IVT), and size and quality were confirmed by electrophoresis (data not shown). Two different versions of the targets were used: a) A short sequence of 60 nucleotides length that contained the 23 nt spacer/target sequence precisely in the middle, and b) a longer version consisting of a partial section of the target gene sequence where one or several 23-nt spacers were located in different positions along the target. The short targets were commercially synthesized, while the long targets were IVT.

Studies sought to evaluate the stability of ribonucleoprotein complexes at high concentration of the complex itself and against three typical components of harsh buffers (like those required for lysis of viral capsids): a) pH, b) Detergent, c) Salt concentration. Four buffers were defined and evaluated protein stability in them by observing protein precipitation. Under all conditions, dEsCas13d was the most stable effector with no signs of precipitation in any of the conditions tested. The other three dCas proteins were sensitive to low salt concentrations with moderate to high precipitation. In general, all Cas proteins are were stable at high salt, 7.4 pH, and either 0.01% Igepal or 1% Triton. The only exception was apo-dLwaCas13a, which precipitated within buffer containing Triton.

### Electrochemical Characterization of OECT Biofunctionalization

Subsequent studies evaluated whether gate electrode biofunctionalization directly affects the sensitivity and general functionality of the OECT. Good coverage of the receptor unit over the gate electrode is necessary but also is a limiting step if a high amount of sample or a long incubation time is required. The coupling of the receptor unit over the gate electrode can be evaluated by electrochemical impedance spectroscopy (EIS). Different combinations of time and concentration of the complex were tested and plotted the impedance spectra of each of these combinations on a Nyquist plot [data now shown]. This plot showed the expected behavior with higher coverage/impedances (semi-circles with larger diameters) at higher concentrations or higher incubation times. 5 μM and 1 h were then selected as the preferred receptor unit concentration and incubation time (respectively) for the biofunctionalization. This condition allows for a high gate coverage while requiring only a moderate Cas-gRNA complex concentration. Studies also characterized the binding of the Cas complex over the surface by monitoring mass increments by Quartz Crystal Microbalance with Dissipation (QCMD) [data not shown]. These experiments showed a stable mass increase after the coupling of the dCas to the sensor surface for all four Cas effectors.

First OECT Sensing Experiments: Yeast tRNA and Igepal in Binding Buffer Reduce Noise and Increase Selectivity

Next, the normalized response (NR) at different target or negative control concentrations was analyzed to characterize the target-binding over the sensor by cyclic voltammetry (CV). First, 10 min was set as the target incubation time required for a moderate signal. However, the data showed that both sensitivity and selectivity required to be improved. A new binding buffer (WBB3) was formulated, enriched with yeast tRNA, higher salt, and a different detergent (Igepal CA-63 instead of Tween-20). The new formulation resulted in higher selectivity and sensitivity.

Binding is Conserved in High Detergent Concentration, Whereas Affinity Depends on the Length of the Target and the Presence of Yeast tRNA

In parallel to the OECT characterization efforts, subsequent studies biochemically characterized the binding performance of the candidate Cas effectors in solution. Electrophoretic mobility shift assays (EMSA) confirm the selectivity of both dSauCas9 and dEsCas13d to the target E2 [data not shown]. Interestingly, in both cases, a heavier band was observed at 0.5 μM of the dSauCas9:gRNA and 2 μM of the dEsCas13d: gRNA. While not being bound by theory, two possible explanations are: a) the complex multimerizes at high concentrations, or b) there is a heterogenous conformation pattern of the ternary complex that moves slower in the non-denaturing electrophoresis. The equilibrium binding affinity of the different Cas effectors was studied by microscale thermophoresis (MST). First, Cas effectors were combined when binding a short target (from gene E2) and a short negative control (from gene N2) in binding buffer without yeast tRNA. In this experiment, dSauCas9 and dEsCas13d showed the highest selectivity and stronger affinity to the target (˜10 nM). Likewise, dLwaCas13a showed a high selectivity but a nine times weaker affinity. Unlike the other three Cas effectors, dLbuCas13a bound both target and negative control with similar affinity, although there was a difference in the direction of the binding curves (data not shown).

Based on their expression yield, SEC-MALS characterization, and target-binding performance, dSauCas9 and dEsCas13d were selected for further analysis.

Since secondary structure is more likely occurring in longer RNA molecules, studies were conducted to evaluated the affinity of dSauCas9 and dEsCas13d against the longer targets in binding buffer again without yeast tRNA [data not shown]. Surprisingly, the performance of both effectors decreased. In both cases, the MST did not reach the response amplitude observed with the short targets (half of dSauCas9). More importantly, dSauCas9 now showed unspecific binding to the negative control with an affinity comparable to target binding.

dSauCas9 and dEsCas13d recovered their specificity towards long RNA targets and also increased the MST response amplitude increased in the presence of yeast tRNA [data not shown]. However, yeast tRNA blocking also somewhat reduced the binding affinity for the target. Thus yeast tRNA can block the interaction between non-target RNA and the Cas:gRNA complex but also competes, to some extent, with the interaction between target and Cas complex.

A binding competition assay was performed to examine how the yeast tRNA interferes with the target binding to the complex. In this experiment, the concentration of yeast tRNA was variable while the concentration of the ternary complex dSauCas9:guideRNA:target/negative control remained constant [data not shown]. Yeast tRNA bound to both the complex with target or negative control RNA (and presumably outcompeted both target and non-target binding). However, the observed half maximal effective concentration (EC50) differed in presence of on-target or non-target RNA:nonNon-target RNA was more easily replaced leading to a three-fold smaller EC50 value. In conclusion, tRNA can serve as an effective blocking agent to prevent unspecific binding to the Cas complex. 4 μM was used as the concentration of yeast tRNA in the new binding buffer. This concentration maximizes its blocking activity (due to a smaller EC50 (6.4 μM) required against the negative control) while still minimizing competition with target binding.

dSauCas9 and dEsCas1 3d Target Binding is Conserved in Lysis Buffer

The aim was to perform experiments in raw saliva samples. For this a lysis buffer is required. Lysis buffers usually contain detergents at high concentration that could be destructive or inhibitory for proteins. The compatibility of the dSauCas9 and dEsCas13d binding systems in a binding buffer containing high concentration of detergent (1% Triton) was tested. The MST experiment was repeated in the simplest setup (against the short targets and in the absence of yeast tRNA but in the presence of this high detergent concentration) to directly inquire the effect of detergent in the protein performance. The MST experiment confirmed the stability and selectivity of the studied receptor units. Interestingly, the binding performance affinity of the Cas effectors against the target RNA in a buffer with 1% Triton (KD of 6.3 nM (±1.3) for dSauCas9 and 0.8 nM (±0.5) for dEsCas13d) was stronger than the previous assessment [data not shown] with only 0.5% Igepal (KD of 9.5 nM (±1.3) for dSauCas9 and 10.7 nM (±7.7) for dEsCas13d).

dEsCas13d was the Most Sensitive and Specific Cas Effector in Preliminary OECT Experiments

Four Cas effector proteins were tested on OECT sensors. The longer version of the SARS-Cov2 Gene E2 was used as a target and the Human gene RPP30 as a negative control. Experiments were performed in lysis buffer (with 1% triton) and in a mixture (4:1) of lysis buffer and raw saliva (with the target or negative control spiked in). The performance in pure buffer was generally less noisy than in buffer and saliva. dEsCas13d and dLwaCas13a stood out for their reproducibility, sensitivity, and selectivity.

### Discussion

Most Cas-based RNA detection methods use the (indirect) collateral cleavage of Cas proteins reporter RNA molecules to report target detection Some sensors are only capable of detecting DNA, cannot measure oligonucleotides in raw samples, requires additional steps to achieve low noise and, because it uses and an OFET instead of an OECT to amplify the target-binding signal, operates at a relatively higher voltage. The disclosed devices and methods combines the programmable binding of CRISPR-Cas proteins to target RNA with the large signal amplification of OECTs in order to rapidly, reliably, and sensitively detect RNA in untreated samples.

Unlike other CRISPR-Cas based approaches that rely mainly on a are generally optimized towards a single Cas effector the disclosed modular design allows evaluation of the performance of four different RNA-targeting Cas effectors in parallel. As expected, the proteins' individual mechanisms and features affect their stability, biochemical binding, and sensing performance. The data showed that dEsCas13d:guide RNA complex is a very stable complex that and does not precipitate even at high concentrations in a buffer with a harsh detergent

Even though preliminary, the OECT sensing experiments and MST results showed a clear dependence benefit of yeast tRNA to increase the selectivity of binding to longer targets.

The present studies biophysically characterize four Cas proteins, their stability as binary complexes, their binding activity in solution and when immobilized on the surface of the disclosed OECT sensors. Indeed, one of the advantages of the disclosed multi-modular sensor platform has been the flexibility to test the performance of four different receptor units under similar conditions.

The OECT results attain a limit of detection in the high attomolar to femtomolar range in lysis buffer and saliva in about 15 min from sample to result. There is no need for sample pre-treatment. The sample volume is less than 10 μL. The tests with dEsCas13d receptor unit indicate good guide RNA-dependent selectivity.
[8] M. Park, et al., Biosensors and Bioelectronics 2010, 26, 1297.
[9] S. Okamoto, et al., Japanese Journal of Applied Physics 2012, 51, 06FD08.
[10] D.-J. Kim, et al., Biosensors and bioelectronics 2013, 41, 621.
[11] C. I. Justino, et al., Talanta 2013, 108, 165.
[12] I.-K. Lee, et al., Nanoscale 2015, 7, 16789.
[13] T. Minamiki, et al., Sensors 2016, 16, 2033.
[14] P. L. Chiang, et al., Chemistry—An Asian Journal 2012, 7, 2073.
[15] Y. D. Ivanov, et al., Lab on a Chip 2012, 12, 5104.
[16] N. Lu, et al., ACS applied materials & interfaces 2014, 6, 20378.
[17] Y. Zhang, et al., Analytical Sciences 2015, 31, 73.
[18] N. Lu, et al., Analytical chemistry 2015, 87, 11203.
[19] Y.-W. Huang, et al., Analytical chemistry 2013, 85, 7912.
[20] K. Zhu, et al., Sensors 2015, 15, 19225.
[21] T. Kong, et al., Biosensors and Bioelectronics 2012, 34, 267.
[22] K. Kim, et al., Biosensors and Bioelectronics 2016, 77, 695.
[23] A. Sharma, et al., Analytica chimica acta 2015, 869, 68.
[24] C. Tlili, et al., Biosensors and Bioelectronics 2011, 26, 4382.
[25] M. B. Lerner, et al., Biosensors and Bioelectronics 2013, 45, 163.
[26] O. S. Kwon, et al., Advanced Materials 2013, 25, 4177.
[27] T. Minamiki, et al., Polymer Journal 2019, 51, 1.
[28] X. Jin, et al., Microchimica Acta 2019, 186, 1.
[29] T. Minamiki, et al., Materials 2014, 7, 6843.
[30] N. M. Andoy, et al., Advanced Materials Technologies 2018, 3, 1800186.
[31] S. Ricci, et al., Biosensors and Bioelectronics 2020, 167, 112433.
[32] P. Seshadri, et al., Biosensors and Bioelectronics 2018, 104, 113.
[33] J. Peng, et al., Microchimica Acta 2018, 185, 1.
[34] W. Hai, et al., Sensors and Actuators B: Chemical 2018, 260, 635.
[35] D. Gentili, et al., Journal of Materials Chemistry B 2018, 6, 5400.
[36] M. Magliulo, et al., Analytical and bioanalytical chemistry 2016, 408, 3943.
[37] M. Berto, et al., Sensors and Actuators B: Chemical 2019, 281, 150.
[38] D.-J. Kim, et al., Biosensors and Bioelectronics 2010, 25, 2477.
[39] E. Macchia, et al., presented at 2017 7th IEEE International Workshop on Advances in Sensors and Interfaces (IWASI), 2017.
[40] E. Macchia, et al., Flexible and Printed Electronics 2018, 3, 034002.
[41] E. Macchia, et al., Nature communications 2018, 9, 1.
[42] E. Macchia, et al., Chemistry of Materials 2019, 31, 6476.
[43] G. C. Zaccariotto, et al., Materials 2021, 14, 4230.
[44] G. Ke, et al., Science China Materials 2021, 64, 739.
[45] Y. Peng, et al., Biosensors and Bioelectronics 2021, 186, 113309.
[46] V. J. Vezza, et al., Chemical Communications 2021, 57, 3704.
[47] H. Yousefi, et al., Journal of the American Chemical Society 2021, 143, 1722.
[48] J. A. Zakashansky, et al., Analytical Methods 2021, 13, 874.
[49] T. Apostolou, et al., Journal of Virological Methods 2021, 293, 114166.
[50] L.-V. Kiew, et al., Biosensors and Bioelectronics 2021, 183, 113213.
[51] S. Mavrikou, et al., Sensors 2020, 20, 3121.
[52] M. Kumar, et al., Sensors and Actuators B: Chemical 2021, 343, 130169.
[53] M. Xian, et al., Journal of Vacuum Science & Technology B, Nanotechnology and Microelectronics: Materials, Processing, Measurement, and Phenomena 2021, 39, 033202.
[54] S. Damiati, et al., IEEE Sensors Journal 2021, 21, 13060.
[55] J. Rodriguez-Manzano, et al., ACS central science 2021, 7, 307.
[56] M. D. Torres, et al., Matter 2021, 4, 2403.
[57] J. Li, et al., ACS sensors 2021, 6, 1270.
[58] J. Li, et al., ACS applied materials & interfaces 2021, 13, 22262.
[59] Y. Li, et al., ACS omega 2021, 6, 6643.
[60] A. Idili, et al., ACS sensors 2021, 6, 3093.
[61] J. Li, et al., Biosensors and Bioelectronics 2021, 183, 113206.
[62] G. Seo, et al., ACS nano 2020, 14, 5135.
[63] M. Z. Rashed, et al., Biosensors and Bioelectronics 2021, 171, 112709.
[64] W. Shao, et al., ACS applied materials & interfaces 2021, 13, 10321.
[65] T. Chaibun, et al., Nature communications 2021, 12, 1.
[66] M. Alafeef, et al., ACS nano 2020, 14, 17028.
[67] T. Beduk, et al., Analytical chemistry 2021, 93, 8585.
[68] P. Fathi-Hafshejani, et al., ACS nano 2021, 15, 11461.
[69] C. Hwang, et al., Biosensors and Bioelectronics 2021, 185, 113177.

### REFERENCES

Ganser, et al. (2019). Molecular Cell Biology, 20(8), 474-489.
Umuhire et al. (2021). Journal of Biological Chemistry, 296, 100191.
Damase, et al. (2021). Frontiers in Bioengineering and Biotechnology, 9, 628137. https://doi.org/10.3389/fbioe.2021.628137
Zhang, et al. (2021). Drug Design, Development and Therapy, 15, 721-733.
Brister, et al. (2015). Nucleic Acids Research, 43(D1), D571-D577.
Woolhouse, et al. (2013). Future Virology, 8(2), 159-171.
Woolhouse, et al. (2016). Emerging Infectious Diseases, 22(12), 2037-2044.
Cucinotta, et al. (2020). Acta Bio Medica: Atenei Parmensis, 91(1), 157-160.
Dong, et al. (2020). The Lancet Infectious Diseases, 20(5), 533-534.
Liu, et al. (2020). Biotechnology Advances, 39, 107463.
Lv, et al. (2020). The Science of the Total Environment, 742, 140370.
Nemati, et al. (2016). Advanced Pharmaceutical Bulletin, 6(3), 301-308.
Akagi, et al. (2020). BMC Infectious Diseases, 20(1), 281.
Trypsteen, et al. (2020). 16(10), e1009037.
Dao et al. (2020). Science Translational Medicine, 12(556), eabc7075.
Esbin, et al. (2020). RNA, 26(7), 771-783.
Notomi, et al. (2000). Nucleic Acids Research, 28(12), e63.
Wang, et al. (2020). Small, 16(32), 2002169. https://doi.org/10.1002/smll.202002169
Sule, et al. (2020). The Pan African Medical Journal, 35(Suppl 2), 121.
Dong et al. (2021). Talanta, 224, 121726.
Rabe, et al. (2020). PNAS, 117(39), 24450-24458.
Broughton, et al. (2020). Nature Biotechnology, 38(7), 870-874.
Li, et al. (2018). Cell Discovery, 4(1), 1-4.
Li, et al. b (2019). ACS Synthetic Biology, 8(10), 2228-2237.
Ali, et al. (2020). Virus Research, 288, 198129.
Marsic, et al. (2021) Nano Letters, 21(8), 3596-3603.
Chen, et al. (2018). Science (New York, N.Y.), 360(6387), 436-439.
Gootenberg, et al. (2017). Science (New York, N.Y.), 356(6336), 438-442.
Kumar, et al. (2021) ELife, 10, e67130.
Myhrvold, et al. (2018) Science, 360(6387), 444-448.
Ding, et al. (2020). Nature Communications, 11(1), 4711.
Guo, et al. (2020Cell Discovery, 6(1), 1-4.
Rauch, et al. (2019). Cell, 178(1), 122-134.e12.
Wang, et al. (2020). Science Bulletin, 65(17), 1436-1439.
Hajian, et al. (2019). Nature Biomedical Engineering, 3(6), 427-437. https://doi.org/10.1038/s41551-019-0371-x
Ackerman et al. (2020). Massively multiplexed nucleic acid detection with Cas13. Nature, 582(7811), 277-282.
(2020). Rapid, accurate, nucleobase detection using FnCas9 (p. 2020.09.13.20193581). https://doi.org/10.1101/2020.09.13.20193581
Zhou et al. (2020). Science Advances, 6(39), eabc6246.
Beamish et al. (2019). ACS Sensors, 4(9), 2458-2464.
Hepp et al. (2021). Scientific Reports, 11(1), 19579.
Hunt, et al. (2022). New Biotechnology, 66, 53-60.
Corman, et al. (2020). Eurosurveillance, 25(3), 2000045.
Takahashi, et al. (2021). ACS Omega, 6(11), 7374-7386.
Aoki et al. (2021). Scientific Reports, 11(1), 9026.
Palaz, et al. (2021) Clinical Biochemistry, 89, 1-13.
Ishino, et al. (1987). Journal of Bacteriology, 169(12), 5429-5433.
Mojica, et al. (1993). Molecular Microbiology, 9(3), 613-621.
Mojica et al. (2000). Molecular Microbiology, 36(1), 244-246.
Jinek et al. (2012). Science (New York, N.Y.), 337(6096), 816-821.
Cong, et al. (2013). Science, 339(6121), 819-823.
Mali, et al. (2013). Science, 339(6121), 823-826.
Makarova, et al. (2020). Nature Reviews Microbiology, 18(2), 67-83.
(2018). A low-cost paper-based synthetic biology platform for analyzing gut microbiota and host biomarkers. Nature Communications, 9(1). https://doi.org/10.1038/s41467-018-05864-4
Verosloff, et al. (2019). ACS Synthetic Biology, 8(4), 902-905. https://doi.org/10.1021/acssynbio.8b00526
Jansen, et al. (2021) PLOS ONE, 16(8), e0256378.
Strakosas, et al. (2015). Journal of Applied Polymer Science, 132(15).
Paudel, et al. (2021). Journal of Materials Chemistry C, 9(31), 9761-9790.
Macchia, et al. (2020). Materials Horizons, 7(4), 999-1013.
Guo, et al. (2021). Nature Biomedical Engineering, 5(7), 666-677
Nguyen, et al. (2019). Materials, 12(1), 121.
Friedlein, et al. (2018). Organic Electronics, 63, 398-414.
Torricelli, et al. (2021). Electrolyte-gated transistors for enhanced performance bioelectronics. Nature Reviews Methods Primers, 1(1), 1-24. https://doi.org/10.1038/s43586-021-00065-8
Puig, et al. (2021). Simultaneous detection of SARS-CoV-2 RNA and host antibodies enabled by a multiplexed electrochemical sensor platform (p. 2021.09.01.21262387). https://doi.org/10.1101/2021.09.01.21262387
Li, et al. (2021). Analytical Chemistry, 93(46), 15501-15507
Zakeri, et al. (2012). 109(12), E690-E697.
Tambe, et al. (2018). Cell Reports, 24(4), 1025-1036.
Abudayyeh, et al. (2016). Science, 353(6299).
Konermann, et al. (2018). Cell, 173(3), 665-676.e14.
Nishimasu, et al. (2015). Cell, 162(5), 1113-1126.
Abudayyeh, et al. (2016). Science, 353(6299).
Andrews et al. (2020). BioRxiv, 2020.04.17.045161.
Strutt, et al. (2018). ELife, 7, e32724.
Abudayyeh, et al. (2017). Nature, 550(7675), 280-284.
East-Seletsky, et al. (2017). Molecular Cell, 66(3), 373-383.e3.
Zhou, et al. (2021). Laboratory Investigation, 1-10.
Yuan, et al. (2021) ACS Synthetic Biology, 10(11), 2824-2832.
Charles et al. (2021). Engineering improved Cas13 effectors for targeted post-transcriptional regulation of gene expression (p. 2021.05.26.445687). https://doi.org/10.1101/2021.05.26.445687
Ivanov, et al. (2020). Proceedings of the National Academy of Sciences, 117 (11), 5853-5860. https://doi.org/10.1073/pnas. 1913445117
Fozouni, et al. (2021). Cell, 184(2), 323-333.e9.
Shivram, et al. Nature Chemical Biology, 17(1), 10-19.
150. Li, Z., Zhao, W., Ma, S., Li, Z., Yao, Y., & Fei, T. (2021). A Chemical-Enhanced System for CRISPR-Based Nucleic Acid Detection (p. 2021.03.28.437376). https://doi.org/10.1101/2021.03.28.437376
Wustoni, et al. Advanced Materials Interfaces, 6(1), 1800928. https://doi.org/10.1002/admi.201800928
Eshaghi, et al. Angewandte Chemie, 127(47), 14158-14162. https://doi.org/10.1002/ange.201506686

We claim:

1. A biosensor comprising an organic electrochemical transistor (OECT) and a biorecognition layer, wherein the OECT comprises a source electrode, a drain electrode, a channel, and a gate electrode, wherein the source and drain electrodes are electronically connected via the channel, and the gate electrode is removable and is located apart from the source electrode, the drain electrode, and the channel.

2. A biosensor comprising an array of two or more OECTs and a biorecognition layer, wherein the array comprises two or more source electrodes, two or more drain electrodes, two or more corresponding channels, and a common gate electrode, wherein each source electrode pairs with one drain electrode and is electronically connected to the drain electrode via one corresponding channel, and the common gate electrode is removable and is located apart from the source electrodes, the drain electrodes, and the corresponding channels.

3. The biosensor of claim 1 or 2, wherein the biorecognition layer is integrated on the gate electrode or the common gate electrode of the OECT.

4. The biosensor of any one of claims 1-3, wherein the orientation of the biorecognition layer relative to the OECT surface can be represented by the general formula:
L1-AP1:AP2-L2-B Formula I
wherein L1 is a first linker, AP1 is a first peptide binding partner; AP2 is a second peptide partner; AP1 and AP2 are binding partners, L2 is a second linker and B is a biorecognition element, wherein binding of AP1 and AP2 results in a biologically self-assembled monolayer (Bio-SAM), optionally, wherein the L1 comprises an N or C terminal cysteine residue.

5. The biosensor of claim 3 or 4, wherein the biorecognition element is a two-domain or a single-domain antibody fragment, such as a nanobody, or a Cas protein.

6. The biosensor of any one of claims 3-5, wherein the biorecognition element recognizes SAR-CoV-2 receptor binding domain.

7. The biosensor of any one of claims 3-6, wherein the L1 directly attaches to the surface of the gate electrode or common gate electrode.

8. The biosensor of any one of claims 3-7, wherein the L1 comprises SEQ ID NO: 22 or SEQ ID NO:23.

9. The biosensor of any one of claims 3-8, wherein the AP1-AP2 are selected from the group consisting of SpyTag/Spy Catcher peptide conjugate, snoopCatcher/snoop Tag, Moon Tag/MoonCatcher. SnoopTagJr/SnoopCatcher or DogTag and SdyTag/SdyCatcher peptide conjugate.

10. The biosensor of claim 9, wherein the API comprises CGGSGSGSGAHIVMVDAYKPTK (SEQ ID NO:24) or AHIVMVDAYKPTKGSGC (SEQ ID NO:25).

11. The biosensor of any one of claims 1-10, wherein the channel or each channel comprises a conducting polymer selected from the group consisting of PEDOT:PSS, PEDOT-S, PEDOT: TOS, PEDOTOH:ClO4, PEDOT-co-PEDOTOH:ClO4, P3HT, PTHS, BBL, p(g2T-TT), PTHS-TMA+-co-P3HT, p(gNDI-g2T), p(g0T2-g6T2), P-90, p(g₃C₂T2-T), and p(C₆NDI-T).

12. A method of integrating a biorecognition layer on an electrode comprising:
(i) incubating at least a portion of the surface of the electrode with a first incubation solution comprising a first conjugate comprising a first linker and a first peptide binding partner to produce the first peptide binding partner-modified surface via the first linker, and
(ii) incubating the first peptide binding partner-modified surface with a second incubation solution comprising a second conjugate and a blocking agent, wherein the second conjugate comprises a second binding partner and a biorecognition element, wherein the first peptide binding partner conjugates with the second peptide to form a linkage.

13. The method of claim 12, wherein the first peptide binding partner is a SpyTag peptide.

14. The method of claim 12 or 13, wherein the second conjugate is a SpyCatcher-nanobody conjugate.

15. The method of claim 14, wherein the nanobody is a SAR-CoV-2 receptor binding domain binding nanobody.

16. The method of any one of claims 12-15, wherein the blocking agent is BSA.

17. A method of detecting the absence, the presence, or the concentration of an analyte in a biological sample comprising contacting the biological sample with the biosensor of claim 1 or 2, wherein the biosensor further comprises a reservoir, the method comprising:
(v) incubating the gate electrode with the biological sample for a time period sufficient to allow binding between the analyte and the biorecognition element;
(vi) rinsing the gate electrode with a rinsing buffer; and
(vii) measuring a signal ID,
wherein a difference between the signal ID and a background ID is indicative of the absence, the presence, or the concentration of the analyte in the biological sample, and wherein the biological sample is in a liquid form.

18. The method of claim 17, wherein the method further comprises a step of measuring the background ID by:
(i) incubating the gate electrode with a blank solution;
(ii) placing the gate electrode on top of the channel;
(iii) applying a VG and a VD;
(iv) measuring the background ID.

19. The method of claims 17 and 18, wherein the method further comprises a step of adding an electrolyte solution into the reservoir prior to any one of steps (i)-(vii).

20. The method of any one of claims 17-19, wherein steps (v)-(vii) are repeated one or more time.

21. The method of any one of claims 17-20, wherein the biological sample is (a) a bodily fluid selected from the group consisting of whole blood, plasma, serum, saliva, mucus, sputum, bronchial alveolar lavage (BAL), bronchial wash (BW), cerebrospinal fluid (CSF), and urine, or (b) a non-bodily fluid.

22. The method of any one of claims 17-21 further comprising a step of processing a specimen into the biological sample prior to any one of steps (i)-(v), wherein the specimen is selected from the group consisting of tissues, feces, rectal swab, nasopharyngeal swab, and throat swab, optionally, wherein the sample is mixed with a buffer before incubation.

23. The method of any one of claims 17-22 further comprising a step of adding a protease inhibitor into the biological sample prior to any one of steps (i)-(v), wherein the sample is a saliva sample.

24. The method of any one of claims 17-23, wherein the volume of the biological sample is less than 20 μL, less than 10 μL, or less than 5 μL.

25. The method of any one of claims 17-24, wherein the gate electrode is incubated with the biological sample for a time period up to 60 minutes, up to 50 minutes, up to 40 minutes, up to 30 minutes, up to 20 minutes, or up to 10 minutes.

26. An alternating current electrothermal flow (ACET)-enhanced biosensor comprising an organic electrochemical transistor (OECT)-based biosensor and a conductive layer for inducing ACET, wherein the OECT-based biosensor comprises an OECT and a biorecognition layer, wherein the OECT comprises a source electrode, a drain electrode, a channel, and a gate electrode, wherein the source and drain electrodes are electronically connected via the channel, and the gate electrode is removable and is located apart from the source electrode, the drain electrode, and the channel.

27. The ACET-enhanced biosensor of claim 26, wherein the OECT-based biosensor comprises an array of two or more OECTs, wherein the array of OECTs comprises two or more two or more source electrodes, two or more drain electrodes, two or more channels, and one gate electrode, wherein each source electrode pairs with one drain electrode and is electronically connected to the drain electrode via one channel, and the gate electrode is removable and is located apart from the source electrodes, the drain electrodes, and the channels.

28. The ACET-enhanced biosensor of claim 26 or 27, wherein the conductive layer is placed in close proximity to the gate electrode of the OECT.

29. The ACET-enhanced biosensor of any one of claims 26-28, wherein the conductive layer is placed along an edge of the gate electrode and surrounds at least a portion of the gate electrode.

30. The ACET-enhanced biosensor of any one of claims 26-29, wherein the gate electrode is circular in shape, wherein the conductive layer has an arch shape surrounding at least a portion of the circular gate electrode.

31. The ACET-enhanced biosensor of any one of claims 26-30, wherein the conductive layer comprises one or more extension portions configured for contacting a power source that applies an AC potential.

32. The ACET-enhanced biosensor of any one of claims 26-31, wherein the biorecognition layer is integrated on the gate electrode of the OECT.

33. The ACET-enhanced biosensor of any one of claims 26-32, wherein the orientation of the biorecognition layer relative to the OECT surface can be represented by the general formula:
L1-AP1:AP2-L2-B Formula I
wherein L1 is a first linker, AP1 is a first peptide binding partner; AP2 is a second peptide partner; AP1 and AP2 are binding partners, L2 is a second linker and B is a biorecognition element, wherein binding of AP1 and AP2 results in a biologically self-assembled monolayer (Bio-SAM).

34. The ACET-enhanced biosensor of claim 33, wherein the L1 comprises an N or C terminal cysteine residue.

35. The ACET-enhanced biosensor of claim 33 or 34, wherein the L1 directly attaches to the surface of the gate electrode.

36. The ACET-enhanced biosensor of any one of claims 26-32, wherein the orientation of the biorecognition layer relative to the OECT surface can be represented by the general formula:
N-L1-AP1:AP2-L2-B Formula I′
wherein where N is an organic molecules capable of self-assembly to form a first SAM, L1 is a first linker, AP1 is a first peptide binding partner; AP2 is a second peptide partner; AP1 and AP2 are binding partners, L2 is a second linker and B is a biorecognition element, wherein N is chemically conjugated with AP₁, resulting in a chemically self-assembled monolayer (Chem-SAM), and wherein binding of AP1 and AP2 results in a biologically self-assembled monolayer (Bio-SAM).

37. The ACET-enhanced biosensor of claim 36, wherein N is an alkane thiol or derivative thereof.

38. The ACET-enhanced biosensor of any one of claims 33-37, wherein the biorecognition element is a two-domain or a single-domain antibody fragment, such as a nanobody, or a Cas protein.

39. The ACET-enhanced biosensor of any one of claims 33-38, wherein the biorecognition element recognizes SAR-CoV-2 receptor binding domain.

40. The ACET-enhanced biosensor of any one of claims 33-39, wherein the L1 comprises SEQ ID NO: 22 or SEQ ID NO:23.

41. The ACET-enhanced biosensor of any one of claims 33-40, wherein the AP1-AP2 are selected from the group consisting of Spy Tag/Spy Catcher peptide conjugate, snoopCatcher/snoopTag, MoonTag/MoonCatcher. SnoopTagJr/SnoopCatcher or DogTag and SdyTag/SdyCatcher peptide conjugate.

42. The ACET-enhanced biosensor of any one of claims 33-41, wherein the AP1 comprises CGGSGSGSGAHIVMVDAYKPTK (SEQ ID NO:24) or AHIVMVDAYKPTKGSGC (SEQ ID NO:25).

43. The ACET-enhanced biosensor of any one of claims 33-42, wherein the channel or each channel comprises a conducting polymer selected from the group consisting of PEDOT:PSS, PEDOT-S, PEDOT: TOS, PEDOTOH: ClO4, PEDOT-co-PEDOTOH:ClO4, P3HT, PTHS, BBL, p(g2T-TT), PTHS-TMA+-co-P3HT, p(gNDI-g2T), p(g0T2-g6T2), P-90, p(g₃C₂T2-T), and p(C₆NDI-T).

44. A method of detecting the absence, the presence, or the concentration of an analyte in a biological sample comprising contacting the biological sample with the ACET-enhanced biosensor of claim 26, wherein the ACET-enhanced biosensor further comprises a reservoir, the method comprising:
(v) incubating the gate electrode with the biological sample for a time period sufficient to allow binding between the analyte and the biorecognition element;
(vi) rinsing the gate electrode with a rinsing buffer; and
(vii) measuring a signal ID,
wherein a difference between the signal ID and a background ID is indicative of the absence, the presence, or the concentration of the analyte in the biological sample, and wherein the biological sample is in a liquid form.

45. The method of claim 17, wherein prior to and/or during step (v), an AC potential is applied to the conductive layer of the biosensor, wherein the AC potential is sufficient to induce a micro-stirring effect.

46. The method of claim 45, the AC potential is in a range from 1 Vpp to 8 Vpp or from 2 Vpp to 6 Vpp, such as about 6 Vpp (pp refers to peak to peak).

47. The method of any one of claims 44-46, wherein the method further comprises a step of measuring the background ID by:
(i) incubating the gate electrode with a blank solution;
(ii) placing the gate electrode on top of the channel;
(iii) applying a VG and a VD; and
(iv) measuring the background ID,
optionally wherein an AC potential is applied to the conductive layer of the biosensor prior to and/or during step (i), and optionally wherein the AC potential is the same as the AC potential applied to the conductive layer prior to and/or during step (v).

48. The method of any one of claims 44-47, wherein the method further comprises a step of adding an electrolyte solution into the reservoir prior to any one of steps (i)-(vii).

49. The method of any one of claims 44-48, wherein steps (v)-(vii) are repeated one or more time.

50. The method of any one of claims 44-49, wherein the biological sample is (a) a bodily fluid selected from the group consisting of whole blood, plasma, serum, saliva, mucus, sputum, bronchial alveolar lavage (BAL), bronchial wash (BW), cerebrospinal fluid (CSF), and urine, or (b) a non-bodily fluid.

51. The method of any one of claims 44-50 further comprising a step of processing a specimen into the biological sample prior to any one of steps (i)-(v), wherein the specimen is selected from the group consisting of tissues, feces, rectal swab, nasopharyngeal swab, and throat swab, optionally, wherein the sample is mixed with a buffer before incubation.

52. The method of any one of claims 44-51 further comprising a step of adding a protease inhibitor into the biological sample prior to any one of steps (i)-(v), wherein the sample is a saliva sample.

53. The method of any one of claims 44-52, wherein the volume of the biological sample is less than 20 μL, less than 10 μL, or less than 5 μL.

54. The method of any one of claims 44-53, wherein the gate electrode is incubated with the biological sample for a time period up to 5 minutes, up to 4 minutes, up to 3 minutes, up to 2 minutes, or up to 1 minute.

55. The method of any one of claims 44-54, wherein the power consumption is ≤100 nW.
